(19) United States
(12) Patent Application Publication
(10) Pub. No.: US 2024/0294653 A1
(43) Pub. Date: Sep. 5, 2024

(54) ANTI-NR10 ANTIBODY AND USE THEREOF

(71) Applicant: Chugai Seiyaku Kabushiki Kaisha, Tokyo (JP)

(72) Inventors: Taichi Kuramochi, Shizuoka (JP); Keiko Kasutani, Shizuoka (JP); Souhei Ohyama, Shizuoka (JP); Hiroyuki Tsunoda, Shizuoka (JP); Tomoyuki Igawa, Shizuoka (JP); Tatsuhiko Tachibana, Shizuoka (JP); Hirotake Shiraiwa, Shizuoka (JP); Keiko Esaki, Shizuoka (JP)

(73) Assignee: Chugai Seiyaku Kabushiki Kaisha, Tokyo (JP)

(21) Appl. No.: 18/661,829

(22) Filed: May 13, 2024

Related U.S. Application Data
(62) Division of application No. 17/975,865, filed on Oct. 28, 2022.
Application No. 17/975,865 is a division of application No. 16/560,143, filed on Sep. 4, 2019, now abandoned.
Application No. 16/560,143 is a continuation of application No. 14/340,883, filed on Jul. 25, 2014, now abandoned.
Application No. 14/340,883 is a continuation of application No. 12/745,781, filed on Sep. 13, 2010, now abandoned.

(30) Foreign Application Priority Data
Dec. 5, 2007 (JP) 2007-315143
Sep. 26, 2008 (JP) 2008-247425

(51) Int. Cl. C07K 16/28; A61K 39/00; G01N 33/68

(52) U.S. Cl. CPC C07K 16/2866; G01N 33/6869; A61K 2039/505; C07K 2317/24; C07K 2317/56; C07K 2317/565; C07K 2317/567; C07K 2317/76; C07K 2317/92; G01N 2333/7155

(57) ABSTRACT

The present inventors successfully obtained anti-NR10 antibodies having an effective neutralizing activity against NR10. The anti-NR10 antibodies provided by the present invention are useful as, for example, pharmaceuticals for treating or preventing inflammatory diseases.

### CROSS-REFERENCE TO RELATED APPLICATIONS

This application is a divisional of U.S. application Ser. No. 17/975,865, filed Oct. 28, 2022, which is a divisional of U.S. application Ser. No. 16/560,143, filed Sep. 4, 2019 (abandoned), which is a continuation of U.S. application Ser. No. 14/340,883, filed Jul. 25, 2014 (abandoned), which is a continuation of U.S. application Ser. No. 12/745,781, filed Sep. 13, 2010 (abandoned), which is a 371 of PCT/JP2008/072152, filed Dec. 5, 2008, which in turn claims the benefit of Japanese Patent Application Nos. 2007-315143, filed Dec. 5, 2007, and 2008-247425, filed Sep. 26, 2008. The entire content of parent application Ser. No. 17/975,865 is hereby incorporated by reference.

### SEQUENCE LISTING

This application contains a Sequence Listing that has been submitted electronically as an XML file named C1-A0712Y1P-USD4 sq.xml. The XML file, created on Apr. 25, 2024, is 397,543 bytes in size. The material in the XML file is hereby incorporated by reference in its entirety.

### TECHNICAL FIELD The present invention relates to anti-NR10 antibodies, and pharmaceutical compositions comprising an anti-NR10 antibody.

### BACKGROUND ART

Many cytokines are known as humoral factors involved in the growth and differentiation of various types of cells, or in the activation of differentiated mature cell functions. Cytokine-stimulated cells produce different types of cytokines, thereby forming networks of multiple cytokines in the body. Biological homeostasis is maintained by a delicate balance of the mutual regulation between cytokines in these networks. Many inflammatory diseases are thought to result from a failure of such cytokine networks. Thus, monoclonal antibody-based anti-cytokine therapy is drawing much attention. For example, anti-TNF antibodies and anti-IL-6 receptor antibodies have been demonstrated to be highly effective clinically. On the other hand, there are many examples of failure where no therapeutic effects were produced when a single cytokine, such as IL-4, was blocked alone, due to the activation of compensatory pathways in actual pathological conditions.

The present inventors succeeded in isolating a novel cytokine receptor NR10 that was highly homologous to gp130, a receptor for IL-6 signal transduction (Patent Document 1). NR 10 forms a heterodimer with oncostatin M receptor (OSMR) and functions as an IL-31 receptor (Non-patent Document 1). Regarding IL-31, it has been reported that transgenic mice overexpressing IL-31 spontaneously develop pruritic dermatitis (Patent Document 2).

Antibodies that bind to NR 10 and inhibit the binding between NR10 and IL-31 may be effective in treating inflammatory diseases. For clinical use, anti-NR10 antibodies are required to have low immunogenicity. Furthermore, in order to achieve high therapeutic effects, antibodies with strong NR10-binding or neutralizing activity are desired.

Prior art documents of the present invention are described below.

### Patent Document 1: WO00/75314

### Patent Document 2: WO03/060090

Non-patent Document 1: IL-31 is associated with cutaneous lymphocyte antigen-positive skin homing T cells in patients with atopic dermatitis., J Allergy Clin Immunol. 2006 February; 117(2):

418-25.

### DISCLOSURE OF THE INVENTION

### [Problems to be Solved by the Invention]

The present invention was achieved in view of the circumstances described above. An objective of the present invention is to provide anti-NR10 antibodies, and pharmaceutical compositions comprising an anti-NR10 antibody.

### [Means for Solving the Problems]

The present inventors conducted dedicated studies to achieve the objective described above. The present inventors succeeded in obtaining anti-NR10 antibodies having an effective neutralizing activity against NR10. Furthermore, the present inventors succeeded in humanizing the antibodies while maintaining their activity. The present inventors also successfully produced antibodies with improved pharmacokinetics, enhanced antigen-binding activity, improved stability, and/or reduced risk of immunogenicity. These antibodies are useful as therapeutic agents for inflammatory diseases.

The present invention relates to anti-NR10 antibodies, and pharmaceutical compositions comprising an anti-NR 10 antibody. More specifically, the present invention includes:
[1] an antibody that recognizes domain 1 of NR10;
[2] the antibody of [1], which has a neutralizing activity;
[3] the antibody of [1] or [2], which is a humanized antibody;
[4] an anti-NR10 antibody which is any one of:
(1) an antibody comprising a heavy chain variable region which comprises CDR1 comprising the amino acid sequence of SEQ ID NO: 1, CDR2 comprising the amino acid sequence of SEQ ID NO: 2, and CDR3 comprising the amino acid sequence of SEQ ID NO: 3;
(2) an antibody comprising the heavy chain variable region of SEQ ID NO: 4;
(3) an antibody comprising a light chain variable region which comprises CDR1 comprising the amino acid sequence of SEQ ID NO: 5, CDR2 comprising the amino acid sequence of SEQ ID NO: 6, and CDR3 comprising the amino acid sequence of SEQ ID NO: 7;
(4) an antibody comprising the light chain variable region of SEQ ID NO: 8;
(5) an antibody comprising the heavy chain variable region of (1) and the light chain variable region of (3);
(6) an antibody comprising the heavy chain variable region of (2) and the light chain variable region of (4);
(7) an antibody in which one or more amino acids are substituted, deleted, added, and/or inserted in the antibody of any one of (1) to (6), which has an activity equivalent to that of the antibody of any one of (1) to (6); and
(8) an antibody which binds to the same epitope as an epitope bound by the antibody of any one of (1) to (7);
[5] an anti-NR10 antibody which is any one of:
(1) an antibody comprising a heavy chain variable region which comprises CDR1 comprising the amino acid sequence of SEQ ID NO: 9, CDR2 comprising the amino acid sequence of SEQ ID NO: 10, and CDR3 comprising the amino acid sequence of SEQ ID NO: 11;
(2) an antibody comprising the heavy chain variable region of SEQ ID NO: 12;
(3) an antibody comprising a light chain variable region which comprises CDR1 comprising the amino acid sequence of SEQ ID NO: 13, CDR2 comprising the amino acid sequence of SEQ ID NO: 14, and CDR3 comprising the amino acid sequence of SEQ ID NO: 15;
(4) an antibody comprising the light chain variable region of SEQ ID NO: 16;
(5) an antibody comprising the heavy chain variable region of (1) and the light chain variable region of (3);
(6) an antibody comprising the heavy chain variable region of (2) and the light chain variable region of (4);
(7) an antibody in which one or more amino acids are substituted, deleted, added, and/or inserted in the antibody of any one of (1) to (6), which has an activity equivalent to that of the antibody of any one of (1) to (6); and
(8) an antibody which binds to the same epitope as an epitope bound by the antibody of any one of (1) to (7);
[6] an anti-NR10 antibody which is any one of:
(1) an antibody comprising a heavy chain variable region which comprises CDR1 comprising the amino acid sequence of SEQ ID NO: 17, CDR2 comprising the amino acid sequence of SEQ ID NO: 18, and CDR3 comprising the amino acid sequence of SEQ ID NO: 19;
(2) an antibody comprising the heavy chain variable region of SEQ ID NO: 20;
(3) an antibody comprising a light chain variable region which comprises CDR1 comprising the amino acid sequence of SEQ ID NO: 21, CDR2 comprising the amino acid sequence of SEQ ID NO: 22, and CDR3 comprising the amino acid sequence of SEQ ID NO: 23;
(4) an antibody comprising the light chain variable region of SEQ ID NO: 24;
(5) an antibody comprising the heavy chain variable region of (1) and the light chain variable region of (3);
(6) an antibody comprising the heavy chain variable region of (2) and the light chain variable region of (4);
(7) an antibody in which one or more amino acids are substituted, deleted, added, and/or inserted in the antibody of any one of (1) to (6), which has an activity equivalent to that of the antibody of any one of (1) to (6); and
(8) an antibody which binds to the same epitope as an epitope bound by the antibody of any one of (1) to (7);
[7] an anti-NR10 antibody which is any one of:
(1) an antibody comprising a heavy chain variable region which comprises CDR 1 comprising the amino acid sequence of SEQ ID NO: 25, CDR2 comprising the amino acid sequence of SEQ ID NO: 26, and CDR3 comprising the amino acid sequence of SEQ ID NO: 27;
(2) an antibody comprising the heavy chain variable region of SEQ ID NO: 28;
(3) an antibody comprising a light chain variable region which comprises CDR1 comprising the amino acid sequence of SEQ ID NO: 29, CDR2 comprising the amino acid sequence of SEQ ID NO: 30, and CDR3 comprising the amino acid sequence of SEQ ID NO: 31;
(4) an antibody comprising the light chain variable region of SEQ ID NO: 32;
(5) an antibody comprising the heavy chain variable region of (1) and the light chain variable region of (3);
(6) an antibody comprising the heavy chain variable region of (2) and the light chain variable region of (4);
(7) an antibody in which one or more amino acids are substituted, deleted, added, and/or inserted in the antibody of any one of (1) to (6), which has an activity equivalent to that of the antibody of any one of (1) to (6); and
(8) an antibody which binds to the same epitope as an epitope bound by the antibody of any one of (1) to (7);
[8] an antibody or antibody variable region which is any one of:
(1) a heavy chain variable region comprising CDR1 of SEQ ID NO: 196, CDR2 of SEQ ID NO: 197, and CDR3 of SEQ ID NO: 11 (H17);
(2) a heavy chain variable region comprising CDR1 of SEQ ID NO: 176, CDR2 of SEQ ID NO: 197, and CDR3 of SEQ ID NO: 11 (H19);
(3) a heavy chain variable region comprising CDR1 of SEQ ID NO: 196, CDR2 of SEQ ID NO: 197, and CDR3 of SEQ ID NO: 184 (H28, H42);
(4) a heavy chain variable region comprising CDR1 of SEQ ID NO: 9, CDR2 of SEQ ID NO: 197, and CDR3 of SEQ ID NO: 184 (H30, H44);
(5) a heavy chain variable region comprising CDR1 of SEQ ID NO: 176, CDR2 of SEQ ID NO: 197, CDR3 of SEQ ID NO: 184 (H34, H46);
(6) a heavy chain variable region comprising CDR1 of SEQ ID NO: 9, CDR2 of SEQ ID NO: 198, and CDR3 of SEQ ID NO: 184 (H57, H78);
(7) a heavy chain variable region comprising CDR1 of SEQ ID NO: 176, CDR2 of SEQ ID NO: 198, and CDR3 of SEQ ID NO: 184 (H71, H92);
(8) a heavy chain variable region comprising CDR1 of SEQ ID NO: 9, CDR2 of SEQ ID NO: 199, and CDR3 of SEQ ID NO: 184 (H97, H98);
(9) a light chain variable region comprising CDR1 of SEQ ID NO: 200, CDR2 of SEQ ID NO: 170, and CDR3 of SEQ ID NO: 193 (L11);
(10) a light chain variable region comprising CDR1 of SEQ ID NO: 201, CDR2 of SEQ ID NO: 170, and CDR3 of SEQ ID NO: 193 (L12);
(11) a light chain variable region comprising CDR1 of SEQ ID NO: 202, CDR2 of SEQ ID NO: 170, and CDR3 of SEQ ID NO: 193 (L17);
(12) a light chain variable region comprising CDR1 of SEQ ID NO: 203, CDR2 of SEQ ID NO: 170, and CDR3 of SEQ ID NO: 193 (L50);
(13) an antibody comprising the heavy chain variable region of (3) and the light chain variable region of (11);
(14) an antibody comprising the heavy chain variable region of (4) and the light chain variable region of (11);
(15) an antibody comprising the heavy chain variable region of (5) and the light chain variable region of (11);
(16) an antibody comprising the heavy chain variable region of (6) and the light chain variable region of (11);
(17) an antibody comprising the heavy chain variable region of (7) and the light chain variable region of (11);
(18) an antibody comprising the heavy chain variable region of (8) and the light chain variable region of (12);
(19) an antibody in which one or more amino acids are substituted, deleted, added, and/or inserted in the antibody of any one of (13) to (18), which has an activity equivalent to that of the antibody of any one of (13) to (18); and
(20) an antibody which binds to the same epitope as an epitope bound by the antibody of any one of (13) to (18);
[9] an antibody or antibody variable region which is any one of:
(1) a heavy chain variable region comprising the amino acid sequence of SEQ ID NO: 204 (H17);
(2) a heavy chain variable region comprising the amino acid sequence of SEQ ID NO: 205 (H19);
(3) a heavy chain variable region comprising the amino acid sequence of SEQ ID NO: 206 (H28);
(4) a heavy chain variable region comprising the amino acid sequence of SEQ ID NO: 207 (H30);
(5) a heavy chain variable region comprising the amino acid sequence of SEQ ID NO: 208 (H34),
(6) a heavy chain variable region comprising the amino acid sequence of SEQ ID NO: 209 (H42);
(7) a heavy chain variable region comprising the amino acid sequence of SEQ ID NO: 210 (H44);
(8) a heavy chain variable region comprising the amino acid sequence of SEQ ID NO: 211 (H46);
(9) a heavy chain variable region comprising the amino acid sequence of SEQ ID NO: 212 (H57);
(10) a heavy chain variable region comprising the amino acid sequence of SEQ ID NO: 213 (H71);
(11) a heavy chain variable region comprising the amino acid sequence of SEQ ID NO: 214 (H78);
(12) a heavy chain variable region comprising the amino acid sequence of SEQ ID NO: 215 (H92);
(13) a heavy chain variable region comprising the amino acid sequence of SEQ ID NO: 216 (H97);
(14) a heavy chain variable region comprising the amino acid sequence of SEQ ID NO: 217 (H98);
(15) a light chain variable region comprising the amino acid sequence of SEQ ID NO: 218 (L11);
(16) a light chain variable region comprising the amino acid sequence of SEQ ID NO: 219 (L12);
(17) a light chain variable region comprising the amino acid sequence of SEQ ID NO: 220 (L17);
(18) a light chain variable region comprising the amino acid sequence of SEQ ID NO: 221 (L50);
(19) an antibody comprising the heavy chain variable region of (3) and the light chain variable region of (17) (H28L17);
(20) an antibody comprising the heavy chain variable region of (4) and the light chain variable region of (17) (H30L17);
(21) an antibody comprising the heavy chain variable region of (5) and the light chain variable region of (17) (H34L17);
(22) an antibody comprising the heavy chain variable region of (6) and the light chain variable region of (17) (H42L17);
(23) an antibody comprising the heavy chain variable region of (7) and the light chain variable region of (17) (H44L17);
(24) an antibody comprising the heavy chain variable region of (8) and the light chain variable region of (17) (H46L17);
(25) an antibody comprising the heavy chain variable region of (9) and the light chain variable region of (17) (H57L17);
(26) an antibody comprising the heavy chain variable region of (10) and the light chain variable region of (17) (H71L17);
(27) an antibody comprising the heavy chain variable region of (11) and the light chain variable region of (17) (H78L17);
(28) an antibody comprising the heavy chain variable region of (12) and the light chain variable region of (17) (H92L17);
(29) an antibody comprising the heavy chain variable region of (13) and the light chain variable region of (18) (H97L50);
(30) an antibody comprising the heavy chain variable region of (14) and the light chain variable region of (18) (H98L50),
(31) an antibody in which one or more amino acids are substituted, deleted, added, and/or inserted in the antibody of any one of (19) to (30), which has an activity equivalent to that of the antibody of any one of (19) to (30); and
(32) an antibody which binds to the same epitope as an epitope bound by the antibody of any one of (19) to (30);
[10] the anti-NR10 antibody of any one of [4] to [9], which is a humanized antibody;
[11] an antibody, antibody heavy chain, or antibody light chain, which is any one of:
(1) a heavy chain comprising the amino acid sequence of SEQ ID NO: 222 (H17);
(2) a heavy chain comprising the amino acid sequence of SEQ ID NO: 223 (H19);
(3) a heavy chain comprising the amino acid sequence of SEQ ID NO: 224 (H28);
(4) a heavy chain comprising the amino acid sequence of SEQ ID NO: 225 (H30);
(5) a heavy chain comprising the amino acid sequence of SEQ ID NO: 226 (H34);
(6) a heavy chain comprising the amino acid sequence of SEQ ID NO: 227 (H42);
(7) a heavy chain comprising the amino acid sequence of SEQ ID NO: 228 (H44);
(8) a heavy chain comprising the amino acid sequence of SEQ ID NO: 229 (H46);
(9) a heavy chain comprising the amino acid sequence of SEQ ID NO: 230 (H57);
(10) a heavy chain comprising the amino acid sequence of SEQ ID NO: 231 (H71);
(11) a heavy chain comprising the amino acid sequence of SEQ ID NO: 232 (H78);
(12) a heavy chain comprising the amino acid sequence of SEQ ID NO: 233 (H92);
(13) a heavy chain comprising the amino acid sequence of SEQ ID NO: 234 (H97);
(14) a heavy chain comprising the amino acid sequence of SEQ ID NO: 235 (H98);
(15) a light chain comprising the amino acid sequence of SEQ ID NO: 236 (L11);
(16) a light chain comprising the amino acid sequence of SEQ ID NO: 237 (L12);
(17) a light chain comprising the amino acid sequence of SEQ ID NO: 238 (L17);
(18) a light chain comprising the amino acid sequence of SEQ ID NO: 239 (L50);
(19) an antibody comprising the heavy chain of (3) and the light chain of (17) (H28L17);
(20) an antibody comprising the heavy chain of (4) and the light chain of (17) (H30L17);
(21) an antibody comprising the heavy chain of (5) and the light chain of (17) (H34L17);
(22) an antibody comprising the heavy chain of (6) and the light chain of (17) (H42L17);
(23) an antibody comprising the heavy chain of (7) and the light chain of (17) (H44L17);
(24) an antibody comprising the heavy chain of (8) and the light chain of (17) (H46L17);
(25) an antibody comprising the heavy chain of (9) and the light chain of (17) (H57L17);
(26) an antibody comprising the heavy chain of (10) and the light chain of (17) (H71L17);
(27) an antibody comprising the heavy chain of (11) and the light chain of (17) (H78L17);
(28) an antibody comprising the heavy chain of (12) and the light chain of (17) (H92L17);
(29) an antibody comprising the heavy chain of (13) and the light chain of (18) (H97L50);
(30) an antibody comprising the heavy chain of (14) and the light chain of (18) (H98L50);
(31) an antibody in which one or more amino acids are substituted, deleted, added, and/or inserted in the antibody of any one of (19) to (30), which has an activity equivalent to that of the antibody of any one of (19) to (30); and
(32) an antibody which binds to the same epitope as an epitope bound by the antibody of any one of (19) to (30);
[12] a pharmaceutical composition comprising the antibody of any one of [1] to [11];
[13] the pharmaceutical composition of [12], which is an agent for treating an inflammatory disease;
[14] a method for treating or preventing an inflammatory disease, which comprises the step of administering the antibody of any one of [1] to [11]; and
[15] use of the antibody of any one of [1] to in the preparation of a therapeutic agent for an inflammatory disease.

### BRIEF DESCRIPTION OF THE DRAWINGS

FIG. 1 shows the amino acid sequences of the heavy chain variable regions of mouse antibodies NS18, NS22, NS23, and NS33.

FIG. 2 shows the amino acid sequences of the light chain variable regions of mouse antibodies NS18, NS22, NS23, and NS33.

FIG. 3 is a graph showing the inhibition of hNR 10/hOSMR/BaF3 cell growth by hybridoma culture supernatants.

FIG. 4 is a graph showing the inhibition of cynNR10/cynOSMR/BaF3 cell growth by hybridoma culture supernatants.

FIG. 5 is a graph showing the assessment of the activity of chimeric NS22 (BaF).

FIG. 6 is a graph showing the assessment of the activity of chimeric NS22 (DU-145).

FIG. 7 is a graph showing the assessment of the competition of chimeric NS22 with IL-31.

FIG. 8 is a graph showing the NR10 competitive binding activity of anti-NR10 antibodies.

FIG. 9 is a set of graphs showing the assessment of the competition of humanized NS22 (H0L0) with IL-31.

FIG. 10 shows the effect of the constant region of humanized anti-NR10 antibody H0L0 on the heterogeneity assessed by cation exchange chromatography.

FIG. 11 is a set of graphs showing the assessment of the competition of mutants of the humanized anti-NR10 antibody of which the isoelectric point of the variable regions is lowered without significant loss of the binding to NR10, with IL-31.

FIG. 12 shows the effect of the constant region of anti-IL-6 receptor antibody on the heterogeneity assessed by cation exchange chromatography.

FIG. 13 shows the effect of the constant region of anti-IL-6 receptor antibody on the denaturation peak assessed by DSC.

FIG. 14 shows the effect of the novel constant region M14 on the heterogeneity in an anti-IL-6 receptor antibody, assessed by cation exchange chromatography.

FIG. 15 shows the effect of the novel constant region M58 on the heterogeneity in an anti-IL-6 receptor antibody, assessed by cation exchange chromatography.

FIG. 16 shows the effect of the novel constant region M58 on the denaturation peak in an anti-IL-6 receptor antibody, assessed by DSC.

FIG. 17 shows the result of assaying the retention of huPM1-IgG1 and huPM1-M58 in the plasma of human FcRn transgenic mice.

FIG. 18 shows the biological activity of each antibody assessed using BaF/NR10.

FIG. 19 shows the analysis of thermally-accelerated (dotted line) and non-accelerated (solid line) samples of each modified antibody by cation exchange chromatography to compare the generation of degradation products between before and after thermal acceleration. Arrow indicates the peak position of basic component which was altered.

FIG. 20 is a set of graphs showing the assessment (BaF) of the activity of each variant.

FIG. 21 is a graph showing the assessment (BaF) of the activity of Ha401La402 and H0L0.

FIG. 22 is a graph showing the assessment (BaF) of the activity of H17L11 and H0L0.

FIG. 23 is a graph showing the assessment (BaF) of the activity of H19L12 and H0L0.

FIG. 24 is a graph showing the biological activity of H0L12 and H0L17 assessed using BaF/NR10.

FIG. 25-1 is a set of graphs showing the assessment (BaF) of the activity of each variant. FIG. 25-2 is a continuation of FIG. 25-1.

FIG. 26 is a schematic diagram for human/mouse wild-type and chimeric NR10-ECD.

FIG. 27 is a set of photographs showing the detection of the binding domain by Western blotting. A is a photograph showing the result of detection using a humanized anti-human NR10 antibody; B is a photograph showing the result of detection using a mouse anti-human NR10 antibody; and C is a photograph showing the result of detection using an anti-Myc antibody. With the anti-human NR10 antibody a binding antigen was detected only in hhh, hhm, and hmm, but not in mmm, mmh, and mhm.

FIG. 28-1 shows the amino acid sequence of each variant of H0 (SEQ ID NO: 50).

FIG. 28-2 is a continuation of FIG. 28-1.

FIG. 28-3 is a continuation of FIG. 28-2.

FIG. 29-1 shows the amino acid sequence of each variant of L0 (SEQ ID NO: 52).

FIG. 29-2 is a continuation of FIG. 29-1.

### MODE FOR CARRYING OUT THE INVENTION

### NR10

NR 10 is a protein that forms a heterodimer with oncostatin M receptor (OSMR) and functions as an IL-31 receptor. NR10 is also known as glm-r (J Biol Chem 277, 16831-6, 2002), GPL (J Biol Chem 278, 49850-9, 2003), IL31RA (Nat Immunol 5, 752-60, 2004), and such. Thus, NR10 in the present invention also includes proteins called by such names.

In the present invention, NR10 (also referred to as IL31RA, GPL, or glm-r) is not particularly limited in terms of its origin, and includes those derived from humans, mice, monkeys, and other mammals. NR10 derived from humans, mice, and monkeys is preferred, and human-derived NR 10 is particularly preferred.

There are multiple known splicing variants of human-derived NR10 (WO 00/075314). Of the above-described splicing variants, NR10.1 consists of 662 amino acids and contains a transmembrane domain. NR10.2 is a soluble receptor-like protein consisting of 252 amino acids without the transmembrane domain. Meanwhile, known NR10 splicing variants that function as transmembrane receptor proteins include NR10.3 and IL-31RAv3. The human NR 10 of the present invention is not particularly limited, as long as it forms a heterodimer with oncostatin M receptor (OSMR) and functions as an IL-31 receptor. Preferred NR10 includes NR10.3 (also referred to as ILRAv4 (Nat Immunol 5, 752-60, 2004)) and IL-31RAv3. NR 10.3 (IL31RAv4) consists of 662 amino acids (WO 00/075314; Nat Immunol 5, 752-60, 2004) and IL31RAv3 consists of 732 amino acids (GenBank Accession No: NM_139017). The amino acid sequence of IL31RAv4 is shown in SEQ ID NO: 79, and the amino acid sequence of

IL31RAv3 is shown in SEQ ID NO: 80. Meanwhile, mouse-derived NR 10 includes proteins comprising the amino acid sequence of SEQ ID NO: 81. In addition, cynomolgus monkey-derived NR10 includes proteins comprising the amino acid sequence of SEQ ID NO: 66.

### Antibodies (Sequences)

Preferred embodiments of the anti-NR 10 antibody of the present invention include the anti-NR10 antibodies of any one of (1) to (8) in (A) to (D) below.

### (A)NS18

(1) antibodies having a heavy chain variable region that comprises CDR1 having the amino acid sequence of SEQ ID NO: 1 (HCDR1), CDR2 having the amino acid sequence of SEQ ID NO: 2 (HCDR2), and CDR3 having the amino acid sequence of SEQ ID NO: 3 (HCDR3);
(2) antibodies having the heavy chain variable region of SEQ ID NO: 4 (VH);
(3) antibodies having a light chain variable region that comprises CDR1 having the amino acid sequence of SEQ ID NO: 5 (LCDR1), CDR2 having the amino acid sequence of SEQ ID NO: 6 (LCDR2), and CDR3 having the amino acid sequence of SEQ ID NO: 7 (LCDR3); (4) antibodies having the light chain variable region of SEQ ID NO: 8 (VL);
(5) antibodies having the heavy chain variable region of (1) and the light chain variable region of (3);
(6) antibodies having the heavy chain variable region of (2) and the light chain variable region of (4);
(7) antibodies in which one or more amino acids are substituted, deleted, added, and/or inserted in the antibodies of any one of (1) to (6), which have an activity equivalent to that of the antibodies of any one of (1) to (6); and
(8) antibodies that bind to the same epitope as an epitope bound by the antibodies of any one of (1) to (7).

### (B) NS22

(1) antibodies having a heavy chain variable region that comprises CDR1 having the amino acid sequence of SEQ ID NO: 9 (HCDR1), CDR2 having the amino acid sequence of SEQ ID NO: 10 (HCDR2), and CDR3 having the amino acid sequence of SEQ ID NO: 11 (HCDR3);
(2) antibodies having the heavy chain variable region of SEQ ID NO: 12 (VH);
(3) antibodies having a light chain variable region that comprises CDR1 having the amino acid sequence of SEQ ID NO: 13 (LCDR1), CDR2 having the amino acid sequence of SEQ ID NO: 14 (LCDR2), and CDR3 having the amino acid sequence of SEQ ID NO: 15 (LCDR3);
(4) antibodies having the light chain variable region of SEQ ID NO: 16 (VL);
(5) antibodies having the heavy chain variable region of (1) and the light chain variable region of (3);
(6) antibodies having the heavy chain variable region of (2) and the light chain variable region of (4);
(7) antibodies in which one or more amino acids are substituted, deleted, added, and/or inserted in the antibodies of any one of (1) to (6), which have an activity equivalent to that of the antibodies of any one of (1) to (6); and
(8) antibodies that bind to the same epitope as an epitope bound by the antibodies of any one of (1) to (7).

Specific examples of the above-described substitution, deletion, addition, and/or insertion of one or more amino acids are not particularly limited and include, for example, the following modifications.

Substitution of Ile at position 3 in the heavy chain CDR1 of SEQ ID NO: 9 with another amino acid. The amino acid after substitution is not particularly limited but preferred examples thereof include Val.

Substitution of Met at position 4 in the heavy chain CDR1 of SEQ ID NO: 9 with another amino acid. The amino acid after substitution is not particularly limited but preferred examples thereof include Ile.

Substitution of Met at position 4 in the heavy chain CDR1 of SEQ ID NO: 9 with another amino acid. The amino acid after substitution is not particularly limited but preferred examples thereof include Leu.

Substitution of Ile at position 3 in the heavy chain CDR1 of SEQ ID NO: 9 with another amino acid. The amino acid after substitution is not particularly limited but preferred examples thereof include Ala.

Substitution of Leu at position 1 in the heavy chain CDR2 of SEQ ID NO: 10 with another amino acid. The amino acid after substitution is not particularly limited but preferred examples thereof include Glu.

Substitution of Asn at position 3 in the heavy chain CDR2 of SEQ ID NO: 10 with another amino acid. The amino acid after substitution is not particularly limited but preferred examples thereof include Asp.

Substitution of Gln at position 13 in the heavy chain CDR2 of SEQ ID NO: 10 with another amino acid. The amino acid after substitution is not particularly limited but preferred examples thereof include Asp.

Substitution of Lys at position 14 in the heavy chain CDR2 of SEQ ID NO: 10 with another amino acid. The amino acid after substitution is not particularly limited but preferred examples thereof include Gln.

Substitution of Lys at position 16 in the heavy chain CDR2 of SEQ ID NO: 10 with another amino acid. The amino acid after substitution is not particularly limited but preferred examples thereof include Gln.

Substitution of Gly at position 17 in the heavy chain CDR2 of SEQ ID NO: 10 with another amino acid. The amino acid after substitution is not particularly limited but preferred examples thereof include Asp.

Substitution of Lys and Gly at positions 16 and 17, respectively, in the heavy chain CDR2 of SEQ ID NO: 10 with another amino acid. The amino acid after substitution is not particularly limited but preferred examples include substitution of Lys at position 16 with Gln, and Gly at position 17 with Asp.

Substitution of Lys, Lys, and Gly at positions 14, 16, and 17, respectively, in the heavy chain CDR2 of SEQ ID NO: 10 with another amino acid. The amino acid after substitution is not particularly limited but preferred examples include substitution of Lys at position 14 with Gln, Lys at position 16 with Gln, and Gly at position 17 with Asp.

Substitution of Gln, Lys, Lys, and Gly at positions 13, 14, 16, and 17, respectively, in the heavy chain CDR2 of SEQ ID NO: 10 with another amino acid. The amino acid after substitution is not particularly limited but preferred examples include substitution of Gln at position 13 with Asp, Lys at position 14 with Gln, Lys at position 16 with Gln, and Gly at position 17 with Asp.

Substitution of Ser at position 10 in the heavy chain CDR2 of SEQ ID NO: 10 with another amino acid. The amino acid after substitution is not particularly limited but preferred examples thereof include Asp.

Substitution of Gln at position 13 in the heavy chain CDR2 of SEQ ID NO: 10 with another amino acid. The amino acid after substitution is not particularly limited but preferred examples thereof include Pro.

Substitution of Tyr at position 3 in the heavy chain CDR3 of SEQ ID NO: 11 with another amino acid. The amino acid after substitution is not particularly limited but preferred examples thereof include Leu.

Substitution of Met at position 10 in the heavy chain CDR3 of SEQ ID NO: 11 with another amino acid. The amino acid after substitution is not particularly limited but preferred examples thereof include Leu.

Substitution of Asp at position 11 in the heavy chain CDR3 of SEQ ID NO: 11 with another amino acid. The amino acid after substitution is not particularly limited but preferred examples thereof include Glu.

Substitution of Tyr at position 12 in the heavy chain CDR3 of SEQ ID NO: 11 with another amino acid. The amino acid after substitution is not particularly limited but preferred examples thereof include Thr and Ser.

Substitution of Met, Asp, and Tyr at positions 10, 11, and 12, respectively, in the heavy chain CDR3 of SEQ ID NO: 11 with another amino acid. The amino acid after substitution is not particularly limited but preferred examples include substitution of Met at position 10 with Leu, Asp at position 11 with Glu, and Tyr at position 12 with Thr.

Substitution of Asp and Tyr at positions 11 and 12, respectively, in the heavy chain CDR3 of SEQ ID NO: 11 with another amino acid. The amino acid after substitution is not particularly limited but preferred examples include substitution of Asp at position 11 with Glu, and Tyr at position 12 with Thr.

Substitution of Tyr, Asp, and Tyr at positions 3, 11, and 12, respectively, in the heavy chain CDR3 of SEQ ID NO: 11 with another amino acid. The amino acid after substitution is not particularly limited but preferred examples include substitution of Tyr at position 3 with Leu, Asp at position 11 with Glu, and Tyr at position 12 with Thr or Ser.

Substitution of Arg at position 1 in the light chain CDR1 of SEQ ID NO: 13 with another amino acid. The amino acid after substitution is not particularly limited but preferred examples thereof include Gln.

Substitution of Asn at position 5 in the light chain CDR1 of SEQ ID NO: 13 with another amino acid. The amino acid after substitution is not particularly limited but preferred examples thereof include Asp.

Substitution of Arg and Asn at positions 1 and 5, respectively, in the light chain CDR1 of SEQ ID NO: 13 with another amino acid. The amino acid after substitution is not particularly limited but preferred examples include substitution of Arg at position 1 with Gln, and Asn at position 5 with Asp.

Substitution of Ser at position 8 in the light chain CDR1 of SEQ ID NO: 13 with another amino acid. The amino acid after substitution is not particularly limited but preferred examples thereof include Arg.

Substitution of Leu at position 10 in the light chain CDR1 of SEQ ID NO: 13 with another amino acid. The amino acid after substitution is not particularly limited but preferred examples thereof include Val.

Substitution of Ser and Leu at positions 8 and 10, respectively, in the light chain CDR1 of SEQ ID NO: 13 with another amino acid. The amino acid after substitution is not particularly limited but preferred examples include substitution of Ser at position 8 with Arg, and Leu at position 10 with Val.

Substitution of Thr at position 2 in the light chain CDR1 of SEQ ID NO: 13 with another amino acid. The amino acid after substitution is not particularly limited but preferred examples thereof include Ala and Ser.

Substitution of Asn at position 1 in the light chain CDR2 of SEQ ID NO: 14 with another amino acid. The amino acid after substitution is not particularly limited but preferred examples thereof include Asp.

Substitution of Lys at position 3 in the light chain CDR2 of SEQ ID NO: 14 with another amino acid. The amino acid after substitution is not particularly limited but preferred examples thereof include Gln.

Substitution of Leu at position 5 in the light chain CDR2 of SEQ ID NO: 14 with another amino acid. The amino acid after substitution is not particularly limited but preferred examples thereof include Glu.

Substitution of Lys at position 7 in the light chain CDR2 of SEQ ID NO: 14 with another amino acid. The amino acid after substitution is not particularly limited but preferred examples thereof include Gln and Asp.

Substitution of Lys, Leu, and Lys at positions 3, 5, and 7, respectively, in the light chain CDR2 of SEQ ID NO: 14 with another amino acid. The amino acid after substitution is not particularly limited but preferred examples include substitution of Lys at position 3 with Gln, Leu at position 5 with Glu, and Lys at position 7 with Gln.

Substitution of Glu at position 5 in the light chain CDR3 of SEQ ID NO: 15 with another amino acid. The amino acid after substitution is not particularly limited but preferred examples thereof include Asp.

Substitution of Ser at position 6 in the light chain CDR3 of SEQ ID NO: 15 with another amino acid. The amino acid after substitution is not particularly limited but preferred examples thereof include Asp.

Substitution of Thr at position 9 in the light chain CDR3 of SEQ ID NO: 15 with another amino acid. The amino acid after substitution is not particularly limited but preferred examples thereof include Phe.

Each of the above-described substitutions may be made alone, or multiple substitutions may be made in combination. Furthermore, the above substitutions may be combined with other substitutions. These substitutions can improve the antibody pharmacokinetics (retention in plasma), enhance the antigen-binding activity, improve the stability, and/or reduce the risk of immunogenicity.

In the present invention, specific examples of the variable regions having a combination of the above-described substitutions include, for example, heavy chain variable regions having the amino acid sequence of SEQ ID NO: 167 and light chain variable regions having the amino acid sequence of SEQ ID NO: 168. Moreover, examples of the antibodies having a combination of the above-described substitutions include, for example, antibodies that comprise a heavy chain variable region having the amino acid sequence of SEQ ID NO: 167 and a light chain variable region having the amino acid sequence of SEQ ID NO: 168.

Moreover, specific examples of the heavy chain or light chain variable regions having a combination of the above-described substitutions include, for example, the following variable regions:
(a) heavy chain variable regions that comprise CDR1 of SEQ ID NO: 196, CDR2 of SEQ ID NO: 197, and CDR3 of SEQ ID NO: 11 (H17);
(b) heavy chain variable regions that comprise CDR1 of SEQ ID NO: 176, CDR2 of SEQ ID NO: 197, and CDR3 of SEQ ID NO: 11 (H19);
(c) heavy chain variable regions that comprise CDR1 of SEQ ID NO: 196, CDR2 of SEQ ID NO: 197, and CDR3 of SEQ ID NO: 184 (H28, H42);
(d) heavy chain variable regions that comprises CDR1 of SEQ ID NO: 9, CDR2 of SEQ ID NO: 197, and CDR3 of SEQ ID NO: 184 (H30, H44);
(e) heavy chain variable regions that comprise CDR1 of SEQ ID NO: 176, CDR2 of SEQ ID NO: 197, and CDR3 of SEQ ID NO: 184 (H34, H46);
(f) heavy chain variable regions that comprise CDR1 of SEQ ID NO: 9, CDR2 of SEQ ID NO: 198, and CDR3 of SEQ ID NO: 184 (H57, H78);
(g) heavy chain variable regions that comprise CDR1 of SEQ ID NO: 176, CDR2 of SEQ ID NO: 198, and CDR3 of SEQ ID NO: 184 (H71, H92);
(h) heavy chain variable regions that comprise CDR1 of SEQ ID NO: 9, CDR2 of SEQ ID NO: 199, and CDR3 of SEQ ID NO: 184 (H97, H98);
(i) light chain variable regions that comprise CDR1 of SEQ ID NO: 200, CDR2 of SEQ ID NO: 170, and CDR3 of SEQ ID NO: 193 (L11);
(j) light chain variable regions that comprise CDR1 of SEQ ID NO: 201, CDR2 of SEQ ID NO: 170, and CDR3 of SEQ ID NO: 193 (L12);
(k) light chain variable regions that comprise CDR1 of SEQ ID NO: 202, CDR2 of SEQ ID NO: 170, and CDR3 of SEQ ID NO: 193 (L17); and
(1) light chain variable regions that comprise CDR1 of SEQ ID NO: 203, CDR2 of SEQ ID NO: 170, and CDR3 of SEQ ID NO: 193 (L50).

Furthermore, specific examples of the antibodies having a combination of the above-described substitutions include, for example:
(i) antibodies that comprise the heavy chain variable region of (c) and the light chain variable region of (k);
(ii) antibodies that comprise the heavy chain variable region of (d) and the light chain variable region of (k);
(iii) antibodies that comprise the heavy chain variable region of (e) and the light chain variable region of (k);
(iv) antibodies that comprise the heavy chain variable region of (f) and the light chain variable region of (k);
(v) antibodies that comprise the heavy chain variable region of (g) and the light chain variable region of (k); and
(vi) antibodies that comprise the heavy chain variable region of (h) and the light chain variable region of (1).

### (C) NS23

(1) antibodies having a heavy chain variable region that comprises CDR1 having the amino acid sequence of SEQ ID NO: 17 (HCDR1), CDR2 having the amino acid sequence of SEQ ID NO: 18 (HCDR2), and CDR3 having the amino acid sequence of SEQ ID NO: 19 (HCDR3);
(2) antibodies having the heavy chain variable region of SEQ ID NO: 20 (VH);
(3) antibodies having a light chain variable region that comprises CDR1 having the amino acid sequence of SEQ ID NO: 21 (LCDR1), CDR2 having the amino acid sequence of SEQ ID NO: 22 (LCDR2), and CDR3 having the amino acid sequence of SEQ ID NO: 23 (LCDR3);
(4) antibodies having the light chain variable region of SEQ ID NO: 24 (VL);
(5) antibodies having the heavy chain variable region of (1) and the light chain variable region of (3);
(6) antibodies having the heavy chain variable region of (2) and the light chain variable region of (4);
(7) antibodies in which one or more amino acids are substituted, deleted, added, and/or inserted in the antibodies of any one of (1) to (6), which have an activity equivalent to that of the antibodies of any one of (1) to (6); and
(8) antibodies that bind to the same epitope as an epitope bound by the antibodies of any one of (1) to (7).

### (D) NS33

(1) antibodies having a heavy chain variable region that comprise CDR1 having the amino acid sequence of SEQ ID NO: 25 (HCDR1), CDR2 having the amino acid sequence of SEQ ID NO: 26 (HCDR2), and CDR3 having the amino acid sequence of SEQ ID NO: 27 (HCDR3);
(2) antibodies having the heavy chain variable region of SEQ ID NO: 28 (VH);
(3) antibodies having a light chain variable region that comprise CDR1 having the amino acid sequence of SEQ ID NO: 29 (LCDR1), CDR2 having the amino acid sequence of SEQ ID NO: 30 (LCDR2), and CDR3 having the amino acid sequence of SEQ ID NO: 31 (LCDR3);
(4) antibodies having the light chain variable region of SEQ ID NO: 32 (VL);
(5) antibodies having the heavy chain variable region of (1) and the light chain variable region of (3);
(6) antibodies having the heavy chain variable region of (2) and the light chain variable region of (4);
(7) antibodies in which one or more amino acids are substituted, deleted, added, and/or inserted in the antibodies of any one of (1) to (6), which have an activity equivalent to that of the antibodies of any one of (1) to (6); and
(8) antibodies that bind to the same epitope as an epitope bound by the antibodies of any one of (1) to (7).

Any framework regions (FR) may be used for the above-described antibodies of (1) or (3); however, FRs derived from human are preferably used. Furthermore, any constant regions may be used for the above-described antibodies of (1) to (8); however, constant regions derived from human are preferably used. For the antibodies of the present invention, the amino acid sequence of the original FR or constant region may be used without modification, or after being modified to a different amino acid sequence by substitution, deletion, addition, and/or insertion of one or more amino acids.

The amino acid sequence of the heavy chain of the above-described NS18 is shown in SEQ ID NO: 34 and the nucleotide sequence encoding this amino acid sequence is shown in SEQ ID NO: 33. Meanwhile, the amino acid sequence of the light chain is shown in SEQ ID NO: 36 and the nucleotide sequence encoding this amino acid sequence is shown in SEQ ID NO: 35.

The amino acid sequence of the heavy chain of NS22 is shown in SEQ ID NO: 38 and the nucleotide sequence encoding this amino acid sequence is shown in SEQ ID NO: 37. Meanwhile, the amino acid sequence of the light chain is shown in SEQ ID NO: 40 and the nucleotide sequence encoding this amino acid sequence is shown in SEQ ID NO: 39.

The amino acid sequence of the heavy chain of NS23 is shown in SEQ ID NO: 42 and the nucleotide sequence encoding this amino acid sequence is shown in SEQ ID NO: 41.

Meanwhile, the amino acid sequence of the light chain is shown in SEQ ID NO: 44 and the nucleotide sequence encoding this amino acid sequence is shown in SEQ ID NO: 43.

The amino acid sequence of the heavy chain of NS33 is shown in SEQ ID NO: 46 and the nucleotide sequence encoding this amino acid sequence is shown in SEQ ID NO: 45. Meanwhile, the amino acid sequence of the light chain is shown in SEQ ID NO: 48 and the nucleotide sequence encoding this amino acid sequence is shown in SEQ ID NO: 47.

In the present invention, the “activity equivalent to that of the antibody of any one of (1) to (6)” means that the activity of binding and/or neutralizing NR 10 (for example, human NR10) is equivalent. In the present invention, the term “equivalent” means that the activity is not necessarily the same but may be enhanced or reduced as long as the activity is retained.

Antibodies with a reduced activity include, for example, antibodies having an activity that is 30% or more, preferably 50% or more, and more preferably 80% or more of that of the original antibody.

The antibodies of any one of (1) to (6) mentioned above may have a substitution, deletion, addition, and/or insertion of one or more amino acids in the amino acid sequence of the variable regions (CDR sequences and/or FR sequences), as long as the NR10-binding and/or neutralizing activity is retained. Methods well known to those skilled in the art to prepare the amino acid sequence of an antibody that has a substitution, deletion, addition, and/or insertion of one or more amino acids in the amino acid sequence and retains NR10-binding and/or neutralizing activity, include methods for introducing mutations into proteins. For example, those skilled in the art can prepare mutants functionally equivalent to the antibody having NR10-binding and/or neutralizing activity by introducing appropriate mutations into the amino acid sequence of the antibody having NR10-binding and/or neutralizing activity using site-directed mutagenesis (Hashimoto-Gotoh, T, Mizuno, T, Ogasahara, Y, and Nakagawa, M. (1995) An oligodeoxyribonucleotide-directed dual amber method for site-directed mutagenesis. Gene 152, 271-275, Zoller, M J, and Smith, M.(1983) Oligonucleotide-directed mutagenesis of DNA fragments cloned into M13 vectors. Methods Enzymol. 100, 468-500, Kramer, W, Drutsa, V, Jansen, H W, Kramer, B, Pflugfelder, M, and Fritz, H J (1984) The gapped duplex DNA approach to oligonucleotide-directed mutation construction. Nucleic Acids Res. 12, 9441-9456. Kramer W, and Fritz H J (1987) Oligonucleotide-directed construction of mutations via gapped duplex DNA Methods. Enzymol. 154, 350-367, Kunkel, T A (1985) Rapid and efficient site-specific mutagenesis without phenotypic selection. Proc Natl Acad Sci USA. 82, 488-492) or the like. Thus, antibodies that contain one or more amino acid mutations in the variable regions and have NR10-binding and/or neutralizing activity are also included in the antibody of the present invention.

When an amino acid residue is altered, the amino acid is preferably mutated for a different amino acid(s) that conserves the properties of the amino acid side-chain. Examples of amino acid side chain properties are: hydrophobic amino acids (A, I, L, M, F, P, W, Y, and V), hydrophilic amino acids (R, D, N, C, E, Q, G, H, K, S, and T), amino acids containing aliphatic side chains (G, A, V, L, I, and P), amino acids containing hydroxyl group-containing side chains (S, T, and Y), amino acids containing sulfur-containing side chains (C and M), amino acids containing carboxylic acid-and amide-containing side chains (D, N, E, and Q), amino acids containing basic side chains (R, K, and H), and amino acids containing aromatic side chains (H, F, Y, and W) (amino acids are represented by one-letter codes in parentheses). Amino acid substitutions within each group are called conservative substitutions. It is already known that a polypeptide containing a modified amino acid sequence in which one or more amino acid residues in a given amino acid sequence are deleted, added, and/or substituted with other amino acids can retain the original biological activity (Mark, D. F. et al., Proc. Natl. Acad. Sci. USA; (1984) 81:5662-6; Zoller, M. J. and Smith, M., Nucleic Acids Res. (1982) 10:6487-500; Wang, A. et al., Science (1984) 224:1431-3; Dalbadic-McFarland, G. et al., Proc. Natl. Acad. Sci. USA (1982) 79:6409-13). Such mutants have an amino acid identity of at least 70%, more preferably at least 75%, even more preferably at least 80%, still more preferably at least 85%, yet more preferably at least 90%, and most preferably at least 95%, with the variable regions (for example, CDR sequences, FR sequences, or whole variable regions) of the present invention. Herein, sequence identity is defined as the percentage of residues identical to those in the original amino acid sequence of the heavy chain variable region or light chain variable region, determined after the sequences are aligned and gaps are appropriately introduced to maximize the sequence identity as necessary. The identity of amino acid sequences can be determined by the method described below.

Alternatively, the amino acid sequences of variable regions that have a substitution, deletion, addition, and/or insertion of one or more amino acids in the amino acid sequence of the variable regions (CDR sequences and/or FR sequences) and retain NR10-binding and/or neutralizing activity can be obtained from nucleic acids that hybridize under stringent conditions to nucleic acid composed of the nucleotide sequence encoding the amino acid sequence of the variable regions. Stringent hybridization conditions to isolate a nucleic acid that hybridizes under stringent conditions to a nucleic acid that includes the nucleotide sequence encoding the amino acid sequence of the variable regions include, for example, the conditions of 6M urea, 0.4% SDS, 0.5x SSC, and 37° C., or hybridization conditions with stringencies equivalent thereto. With more stringent conditions, for example, the conditions of 6M urea, 0.4% SDS, 0.1x SSC, and 42° C., isolation of nucleic acids with a much higher homology can be expected. The sequences of the isolated nucleic acids can be determined by the known methods described below. The overall nucleotide sequence homology of the isolated nucleic acid is at least 50% or higher sequence identity, preferably 70% or higher, more preferably 90% or higher (for example, 95%, 96%, 97%, 98%, 99%, or higher).

Nucleic acids that hybridize under stringent conditions to a nucleic acid composed of the nucleotide sequence encoding the amino acid sequence of the variable regions can also be isolated using, instead of the above-described methods using hybridization techniques, gene amplification methods such as polymerase chain reaction (PCR) using primers synthesized based on the information of nucleotide sequence encoding the amino acid sequence of the variable regions.

Specifically, the identity of one nucleotide sequence or amino acid sequence to another can be determined using the algorithm BLAST, by Karlin and Altschul (Proc. Natl. Acad. Sci. USA (1993) 90, 5873-7). Programs such as BLASTN and BLASTX were developed based on this algorithm (Altschul et al., J. Mol. Biol. (1990) 215, 403-10). To analyze nucleotide sequences according to BLASTN based on BLAST, the parameters are set, for example, as score=100 and wordlength=12. On the other hand, parameters used for the analysis of amino acid sequences by BLASTX based on BLAST include, for example, score=50 and wordlength=3. Default parameters for each program are used when using the BLAST and Gapped BLAST programs. Specific techniques for such analyses are known in the art (see the website of the National Center for Biotechnology Information (NCBI), Basic Local Alignment Search Tool (BLAST); http://www.ncbi.nlm.nih.gov).

The present invention also provides antibodies that bind to the same epitope as an epitope bound by the antibodies of any one of (1) to (7).

Whether an antibody recognizes the same epitope as that recognized by another antibody can be confirmed by the competition between the two antibodies against the epitope. Competition between the antibodies can be evaluated by competitive binding assays using means such as ELISA, fluorescence energy transfer method (FRET), and fluorometric microvolume assay technology (FMAT(R)). The amount of antibodies bound to an antigen indirectly correlate with the binding ability of candidate competitor antibodies (test antibodies) that competitively bind to the same epitope. In other words, as the amount of or the affinity of test antibodies against the same epitope increases, the amount of antibodies bound to the antigen decreases, and the amount of test antibodies bound to the antigen increases. Specifically, appropriately labeled antibodies and antibodies to be evaluated are simultaneously added to the antigens, and the thus bound antibodies are detected using the label. The amount of antibodies bound to the antigen can be easily determined by labeling the antibodies beforehand. This label is not particularly limited, and the labeling method is selected according to the assay technique used. The labeling method includes fluorescent labeling, radiolabeling, enzymatic labeling, and such.

For example, fluorescently labeled antibodies and unlabeled antibodies or test antibodies are simultaneously added to animal cells expressing NR10, and the labeled antibodies are detected by fluorometric microvolume assay technology.

Herein, the “antibody that recognizes the same epitope” refers to an antibody that can reduce the binding of the labeled antibody by at least 50% at a concentration that is usually 100 times higher, preferably 80 times higher, more preferably 50 times higher, even more preferably 30 times higher, and still more preferably 10 times higher than a concentration at which the non-labeled antibody reduces the binding of the labeled antibody by 50% (IC₅₀).

Antibodies that bind to the epitope to which the antibodies set forth in any one of (1) to (7) above bind are useful because they have a particularly high neutralizing activity.

The antibodies set forth in any one of (1) to (8) above are preferably humanized antibodies, but are not particularly limited thereto.

Furthermore, the present invention provides genes encoding the anti-NR10 antibodies of any one of (1) to (8) of (A) to (D) above. The genes of the present invention may be any form of genes, for example, DNAs or RNAs.

### Antibodies (Humanized)

Preferred embodiments of the antibodies of the present invention include humanized antibodies that bind to NR10. The humanized antibodies can be prepared by methods known to those skilled in the art.

The variable region of antibody is typically composed of three complementarity-determining regions (CDRs) sandwiched by four frames (FRs). The CDRs substantially determine the binding specificity of antibody. The amino acid sequences of CDRs are highly diverse. In contrast, the amino acid sequences of FRs often exhibit high homology between antibodies having different binding specificities. It is therefore said in general that the binding specificity of an antibody can be transplanted to a different antibody by grafting the CDRs.

Humanized antibodies are also referred to as reshaped human antibodies, and they are prepared by transferring the CDRs of an antibody derived from a non-human mammal such as a mouse, to the CDRs of a human antibody. General genetic recombination techniques for their preparation are also known (see European Patent Application Publication No. 125023 and WO 96/02576).

Specifically, for example, when the CDRs are derived from a mouse antibody, a DNA sequence designed such that the CDRs of the mouse antibody are linked with framework regions (FRs) of human antibody is synthesized by PCR using, as primers, several oligonucleotides that have portions overlapping the ends of both CDRs and FRs (see the method described in WO 98/13388). The resulting DNA is then ligated to a DNA encoding a human antibody constant region, inserted into an expression vector, and introduced into a host to produce the antibody (see European Patent Application Publication No. EP 239400 and International Patent Application Publication No. WO 96/02576).

Human antibody framework regions to be linked with CDRs are selected so that the CDRs form a favorable antigen-binding site. If needed, amino acid substitution, deletion, addition, and/or insertion may be introduced into the framework regions of antibody variable region so that the CDRs of the reshaped human antibody form a proper antigen-binding site. For example, mutations can be introduced into the amino acid sequence of FR by applying the

PCR method which is used to graft mouse CDRs to human FRs. Specifically, mutations can be introduced into a portion of the nucleotide sequences of primers that anneal to the FRs. The mutations are introduced into FRs synthesized by such primers. The antigen-binding activity of mutant antibodies having amino acid substitutions can be determined and assessed by the method described above, and thereby mutant FR sequences having desired properties can be selected (Sato, K. et al., Cancer Res. (1993) 53, 851-856).

Constant (C) regions from human antibodies are used for those of humanized antibodies. For example, C□1, C□2, C□3, C□4, C□, C□, C□1, C□2, and C□ are used for H chains; and C□ and C□ are used for L chains. The amino acid sequence of C□ is shown in SEQ ID NO: 58, and the nucleotide sequence encoding this amino acid sequence is shown in SEQ ID NO: 57. The amino acid sequence of C□1 is shown in SEQ ID NO: 60, and the nucleotide sequence encoding this amino acid sequence is shown in SEQ ID NO: 59. The amino acid sequence of C□2 is shown in SEQ ID NO: 62, and the nucleotide sequence encoding this amino acid sequence is shown in SEQ ID NO: 61. The amino acid sequence of C□4 is shown in SEQ ID NO: 64, and the nucleotide sequence encoding this amino acid sequence is shown in SEQ ID NO: 63. Furthermore, human antibody C regions may be modified to improve the stability of antibody or antibody production. Modified human antibody C regions include, for example, the C regions described herein below. Human antibodies used for humanization may be of any isotype such as IgG, IgM, IgA, IgE, or IgD; however, IgG is preferably used in the present invention. IgG that can be used includes IgG1, IgG2, IgG3, IgG4, and the like.

Moreover, after a humanized antibody is prepared, amino acids in the variable region (for example, CDR and FR) and constant region of the humanized antibody may be deleted, added, inserted, and/or substituted with other amino acids. The antibodies of the present invention also include such humanized antibodies with amino acid substitutions and the like.

The origin of CDRs of a humanized antibody is not particularly limited, and may be any animal. For example, it is possible to use the sequences of mouse antibodies, rat antibodies, rabbit antibodies, camel antibodies, and the like. CDR sequences of mouse antibodies are preferred.

In general, it is difficult to humanize antibodies while retaining the binding and neutralizing activities of the original antibodies. The present invention, however, succeeded in obtaining humanized antibodies having the binding and/or neutralizing activities equivalent to those of the original mouse antibodies. Humanized antibodies are useful when administered to humans for the therapeutic purposes, because they exhibit reduced immunogenicity in the human body.

Preferred examples of the humanized anti-NR 10 antibodies of the present invention include, for example:
(a) humanized antibodies that comprise a heavy chain variable region having the amino acid sequence of SEQ ID NO: 50 (H0-VH);
(b) humanized antibodies that comprise a heavy chain variable region having the amino acid sequence of SEQ ID NO: 112 (H1-VH);
(c) humanized antibodies that comprise a light chain variable region having the amino acid sequence of SEQ ID NO: 52 (L0-VL);
(d) humanized antibodies that comprise a heavy chain variable region having the amino acid sequence of SEQ ID NO: 50 (H0-VH) and a light chain variable region having the amino acid sequence of SEQ ID NO: 52 (L0-VL); and
(c) humanized antibodies that comprise a heavy chain variable region having the amino acid sequence of SEQ ID NO: 112 and a light chain variable region having the amino acid sequence of SEQ ID NO: 52.

The heavy chain variable region having the amino acid sequence of SEQ ID NO: 50 (H0-VH), heavy chain variable region having the amino acid sequence of SEQ ID NO: 112, and light chain variable region having the amino acid sequence of SEQ ID NO: 52 (L0-VL) may have a substitution, deletion, addition, and/or insertion of one or more amino acids. The substitution, deletion, addition, and/or insertion of amino acids may be made in either or both of the CDRs and FRs.

Thus, other preferred embodiments of the humanized anti-NR10 antibody of the present invention include, for example:
(f) antibodies that comprise a heavy chain variable region having an amino acid sequence in which one or more amino acids are substituted, deleted, added, and/or inserted in the amino acid sequence of SEQ ID NO: 50 (H0-VH);
(g) antibodies that comprise a heavy chain variable region having an amino acid sequence in which one or more amino acids are substituted, deleted, added, and/or inserted in the amino acid sequence of SEQ ID NO: 112 (H1-VH);
(h) antibodies that comprise a light chain variable region having an amino acid sequence in which one or more amino acids are substituted, deleted, added, and/or inserted in the amino acid sequence of SEQ ID NO: 52 (L0-VL);
(i) antibodies that comprise a heavy chain variable region having an amino acid sequence in which one or more amino acids are substituted, deleted, added, and/or inserted in the amino acid sequence of SEQ ID NO: 50 (H0-VH), and a light chain variable region having an amino acid sequence in which one or more amino acids are substituted, deleted, added, and/or inserted in the amino acid sequence of SEQ ID NO: 52 (L0-VL);
(j) antibodies that comprise a heavy chain variable region having an amino acid sequence in which one or more amino acids are substituted, deleted, added, and/or inserted in the amino acid sequence of SEQ ID NO: 112 (H1-VH), and a light chain variable region having an amino acid sequence in which one or more amino acids are substituted, deleted, added, and/or inserted in the amino acid sequence of SEQ ID NO: 52 (L0-VL);

Without particular limitation, the antibodies of any one of (f) to (j) preferably have an activity similar to that of the antibodies of any one of (a) to (c).

The substitution, deletion, addition, and/or insertion of amino acids are not particularly limited, but specific examples include, for example, the above-described amino acid substitutions.

More specifically, for example, the following amino acid substitutions may be included:

Substitution of Ile at position 3 of CDR1 (SEQ ID NO: 9) in the heavy chain variable region of SEQ ID NO: 50 or 112 with Val (SEQ ID NO: 173). Thus, the present invention provides heavy chain variable regions in which CDR1 having the amino acid sequence of SEQ ID NO: 9 is substituted with CDR1 having the amino acid sequence of SEQ ID NO: 173 in a heavy chain variable region having the amino acid sequence of SEQ ID NO: 50 or 112.

Substitution of Met at position 4 of CDR1 (SEQ ID NO: 9) in the heavy chain variable region of SEQ ID NO: 50 or 112 with Ile (SEQ ID NO: 174). Thus, the present invention provides heavy chain variable regions in which CDR1 having the amino acid sequence of SEQ ID NO: 9 is substituted with CDR1 having the amino acid sequence of SEQ ID NO: 174 in a heavy chain variable region having the amino acid sequence of SEQ ID NO: 50 or 112.

Substitution of Met at position 4 of CDR1 (SEQ ID NO: 9) in the heavy chain variable region of SEQ ID NO: 50 or 112 with Leu (SEQ ID NO: 175). Thus, the present invention provides heavy chain variable regions in which CDR1 having the amino acid sequence of SEQ ID NO: 9 is substituted with CDR1 having the amino acid sequence of SEQ ID NO: 175 in a heavy chain variable region having the amino acid sequence of SEQ ID NO: 50 or 112.

Substitution of Ile at position 3 of CDR1 (SEQ ID NO: 9) in the heavy chain variable region of SEQ ID NO: 50 or 112 with Ala (SEQ ID NO: 176). Thus, the present invention provides heavy chain variable regions in which CDR1 having the amino acid sequence of SEQ ID NO: 9 is substituted with CDR 1 having the amino acid sequence of SEQ ID NO: 176 in a heavy chain variable region having the amino acid sequence of SEQ ID NO: 50 or 112.

Substitution of Leu at position 1 of CDR2 (SEQ ID NO: 10) in the heavy chain variable region of SEQ ID NO: 50 or 112 with Glu (SEQ ID NO: 113). Thus, the present invention provides heavy chain variable regions in which CDR2 having the amino acid sequence of SEQ ID NO: 10 is substituted with CDR2 having the amino acid sequence of SEQ ID NO: 113 in a heavy chain variable region having the amino acid sequence of SEQ ID NO: 50 or 112.

Substitution of Asn at position 3 of CDR2 (SEQ ID NO: 10) in the heavy chain variable region of SEQ ID NO: 50 or 112 with Asp (SEQ ID NO: 114). Thus, the present invention provides heavy chain variable regions in which CDR2 having the amino acid sequence of SEQ ID NO: 10 is substituted with CDR2 having the amino acid sequence of SEQ ID NO: 114 in a heavy chain variable region having the amino acid sequence of SEQ ID NO: 50 or 112.

Substitution of Gln at position 13 of CDR2 (SEQ ID NO: 10) in the heavy chain variable region of SEQ ID NO: 50 or 112 with Asp (SEQ ID NO: 115). Thus, the present invention provides heavy chain variable regions in which CDR2 having the amino acid sequence of SEQ ID NO: 10 is substituted with CDR2 having the amino acid sequence of SEQ ID NO: 115 in a heavy chain variable region having the amino acid sequence of SEQ ID NO: 50 or 112.

Substitution of Lys at position 14 of CDR2 (SEQ ID NO: 10) in the heavy chain variable region of SEQ ID NO: 50 or 112 with Gln (SEQ ID NO: 116). Thus, the present invention provides heavy chain variable regions in which CDR2 having the amino acid sequence of SEQ ID NO: 10 is substituted with CDR2 having the amino acid sequence of SEQ ID NO: 116 in a heavy chain variable region having the amino acid sequence of SEQ ID NO: 50 or 112.

Substitution of Lys at position 16 of CDR2 (SEQ ID NO: 10) in the heavy chain variable region of SEQ ID NO: 50 or 112 with Gln (SEQ ID NO: 117). Thus, the present invention provides heavy chain variable regions in which CDR2 having the amino acid sequence of SEQ ID NO: 10 is substituted with CDR2 having the amino acid sequence of SEQ ID NO: 117 in a heavy chain variable region having the amino acid sequence of SEQ ID NO: 50 or 112.

Substitution of Gly at position 17 of CDR2 (SEQ ID NO: 10) in the heavy chain variable region of SEQ ID NO: 50 or 112 with Asp (SEQ ID NO: 118). Thus, the present invention provides heavy chain variable regions in which CDR2 having the amino acid sequence of SEQ ID NO: 10 is substituted with CDR2 having the amino acid sequence of SEQ ID NO: 118 in a heavy chain variable region having the amino acid sequence of SEQ ID NO: 50 or 112.

Substitution of Lys at position 16 and Gly at position 17 of CDR2 (SEQ ID NO: 10) in the heavy chain variable region of SEQ ID NO: 50 or 112 with Gln and Asp, respectively (SEQ ID NO: 119). Thus, the present invention provides heavy chain variable regions in which CDR2 having the amino acid sequence of SEQ ID NO: 10 is substituted with CDR2 having the amino acid sequence of SEQ ID NO: 119 in a heavy chain variable region having the amino acid sequence of SEQ ID NO: 50 or 112.

Substitution of Lys at position 14, Lys at position 16, and Gly at position 17 of CDR2 (SEQ ID NO: 10) in the heavy chain variable region of SEQ ID NO: 50 or 112 with Gln, Gln, and Asp, respectively (SEQ ID NO: 167). Thus, the present invention provides heavy chain variable regions in which CDR2 having the amino acid sequence of SEQ ID NO: 10 is substituted with CDR2 having the amino acid sequence of SEQ ID NO: 171 in a heavy chain variable region having the amino acid sequence of SEQ ID NO: 50 or 112.

Substitution of Gln at position 13, Lys at position 14, Lys at position 16, and Gly at position 17 of CDR2 (SEQ ID NO: 10) in the heavy chain variable region of SEQ ID NO: 50 or 112 with Asp, Gln, Gln, and Asp, respectively (SEQ ID NO: 172). Thus, the present invention provides heavy chain variable regions in which CDR2 having the amino acid sequence of SEQ ID NO: 10 is substituted with CDR2 having the amino acid sequence of SEQ ID NO: 172 in a heavy chain variable region having the amino acid sequence of SEQ ID NO: 50 or 112.

Substitution of Ser at position 10 of CDR2 (SEQ ID NO: 10) in the heavy chain variable region of SEQ ID NO: 50 or 112 with Asp (SEQ ID NO: 177). Thus, the present invention provides heavy chain variable regions in which CDR2 having the amino acid sequence of SEQ ID NO: 10 is substituted with CDR2 having the amino acid sequence of SEQ ID NO: 177 in a heavy chain variable region having the amino acid sequence of SEQ ID NO: 50 or 112.

Substitution of Gln at position 13 of CDR2 (SEQ ID NO: 10) in the heavy chain variable region of SEQ ID NO: 50 or 112 with Pro (SEQ ID NO: 178). Thus, the present invention provides heavy chain variable regions in which CDR2 having the amino acid sequence of SEQ ID NO: 10 is substituted with CDR2 having the amino acid sequence of SEQ ID NO: 178 in a heavy chain variable region having the amino acid sequence of SEQ ID NO: 50 or 112.

Substitution of Tyr at position 3 of CDR3 (SEQ ID NO: 11) in the heavy chain variable region of SEQ ID NO: 50 or 112 with Leu (SEQ ID NO: 179). Thus, the present invention provides heavy chain variable regions in which CDR3 having the amino acid sequence of SEQ ID NO: 11 is substituted with CDR3 having the amino acid sequence of SEQ ID NO: 179 in a heavy chain variable region having the amino acid sequence of SEQ ID NO: 50 or 112.

Substitution of Met at position 10 of CDR3 (SEQ ID NO: 11) in the heavy chain variable region of SEQ ID NO: 50 or 112 with Leu (SEQ ID NO: 180). Thus, the present invention provides heavy chain variable regions in which CDR3 having the amino acid sequence of SEQ ID NO: 11 is substituted with CDR3 having the amino acid sequence of SEQ ID NO: 180 in a heavy chain variable region having the amino acid sequence of SEQ ID NO: 50 or 112.

Substitution of Asp at position 11 of CDR3 (SEQ ID NO: 11) in the heavy chain variable region of SEQ ID NO: 50 or 112 with Glu (SEQ ID NO: 181). Thus, the present invention provides heavy chain variable regions in which CDR3 having the amino acid sequence of SEQ ID NO: 11 is substituted with CDR3 having the amino acid sequence of SEQ ID NO: 181 in a heavy chain variable region having the amino acid sequence of SEQ ID NO: 50 or 112.

Substitution of Tyr at position 12 of CDR3 (SEQ ID NO: 11) in the heavy chain variable region of SEQ ID NO: 50 or 112 with Thr (SEQ ID NO: 182). Thus, the present invention provides heavy chain variable regions in which CDR3 having the amino acid sequence of SEQ ID NO: 11 is substituted with CDR3 having the amino acid sequence of SEQ ID NO: 182 in a heavy chain variable region having the amino acid sequence of SEQ ID NO: 50 or 112.

Substitution of Tyr at position 12 of CDR3 (SEQ ID NO: 11) in the heavy chain variable region of SEQ ID NO: 50 or 112 with Ser (SEQ ID NO: 183). Thus, the present invention provides heavy chain variable regions in which CDR3 having the amino acid sequence of SEQ ID NO: 11 is substituted with CDR3 having the amino acid sequence of SEQ ID NO: 183 in a heavy chain variable region having the amino acid sequence of SEQ ID NO: 50 or 112.

Substitution of Met at position 10, Asp at position 11, and Tyr at position 12 of CDR3 (SEQ ID NO: 11) in the heavy chain variable region of SEQ ID NO: 50 or 112 with Leu, Glu,

Thr, respectively (SEQ ID NO: 184). Thus, the present invention provides heavy chain variable regions in which CDR3 having the amino acid sequence of SEQ ID NO: 11 is substituted with CDR3 having the amino acid sequence of SEQ ID NO: 184 in a heavy chain variable region having the amino acid sequence of SEQ ID NO: 50 or 112.

Substitution of Asp at position 11 and Tyr at position 12 of CDR3 (SEQ ID NO: 11) in the heavy chain variable region of SEQ ID NO: 50 or 112 with Glu and Thr, respectively (SEQ ID NO: 185). Thus, the present invention provides heavy chain variable regions in which CDR3 having the amino acid sequence of SEQ ID NO: 11 is substituted with CDR3 having the amino acid sequence of SEQ ID NO: 185 in a heavy chain variable region having the amino acid sequence of SEQ ID NO: 50 or 112.

Substitution of Tyr at position 3, Asp at position 11, and Tyr at position 12 of CDR3 (SEQ ID NO: 11) in the heavy chain variable region of SEQ ID NO: 50 or 112 with Leu, Glu, and Thr, respectively (SEQ ID NO: 186). Thus, the present invention provides heavy chain variable regions in which CDR3 having the amino acid sequence of SEQ ID NO: 11 is substituted with CDR3 having the amino acid sequence of SEQ ID NO: 186 in a heavy chain variable region having the amino acid sequence of SEQ ID NO: 50 or 112.

Substitution of Tyr at position 3, Asp at position 11, and Tyr at position 12 of CDR3 (SEQ ID NO: 11) in the heavy chain variable region of SEQ ID NO: 50 or 112 with Leu, Glu, and Ser, respectively (SEQ ID NO: 187). Thus, the present invention provides heavy chain variable regions in which CDR3 having the amino acid sequence of SEQ ID NO: 11 is substituted with CDR3 having the amino acid sequence of SEQ ID NO: 187 in a heavy chain variable region having the amino acid sequence of SEQ ID NO: 50 or 112.

Substitution of Arg at position 1 of CDR1 (SEQ ID NO: 13) in the light chain variable region of SEQ ID NO: 52 with Gln (SEQ ID NO: 121). Thus, the present invention provides light chain variable regions in which CDR1 having the amino acid sequence of SEQ ID NO: 13 is substituted with CDR1 having the amino acid sequence of SEQ ID NO: 121 in a light chain variable region having the amino acid sequence of SEQ ID NO: 52.

Substitution of Asn at position 5 of CDR1 (SEQ ID NO: 13) in the light chain variable region of SEQ ID NO: 52 with Asp (SEQ ID NO: 122). Thus, the present invention provides light chain variable regions in which CDR1 having the amino acid sequence of SEQ ID NO: 13 is substituted with CDR1 having the amino acid sequence of SEQ ID NO: 122 in a light chain variable region having the amino acid sequence of SEQ ID NO: 52.

Substitution of Ser at position 8 of CDR1 (SEQ ID NO: 13) in the light chain variable region of SEQ ID NO: 52 with Arg (SEQ ID NO: 188). Thus, the present invention provides light chain variable regions in which CDR1 having the amino acid sequence of SEQ ID NO: 13 is substituted with CDR1 having the amino acid sequence of SEQ ID NO: 188 in a light chain variable region having the amino acid sequence of SEQ ID NO: 52.

Substitution of Leu at position 10 of CDR1 (SEQ ID NO: 13) of the light chain variable region of SEQ ID NO: 52 with Val (SEQ ID NO: 189). Thus, the present invention provides light chain variable regions in which CDR1 having the amino acid sequence of SEQ ID NO: 13 is substituted with CDR 1 having the amino acid sequence of SEQ ID NO: 189 in a light chain variable region having the amino acid sequence of SEQ ID NO: 52.

Substitution of Ser at position 8 and Leu at position 10 of CDR1 (SEQ ID NO: 13) of the light chain variable region of SEQ ID NO: 52 with Arg and Val, respectively (SEQ ID NO: 190). Thus, the present invention provides light chain variable regions in which CDR1 having the amino acid sequence of SEQ ID NO: 13 is substituted with CDR1 having the amino acid sequence of SEQ ID NO: 190 in a light chain variable region having the amino acid sequence of SEQ ID NO: 52.

Substitution of Thr at position 2 of CDR1 (SEQ ID NO: 13) in the light chain variable region of SEQ ID NO: 52 with Ala (SEQ ID NO: 191). Thus, the present invention provides light chain variable regions in which CDR1 having the amino acid sequence of SEQ ID NO: 13 is substituted with CDR1 having the amino acid sequence of SEQ ID NO: 191 in a light chain variable region having the amino acid sequence of SEQ ID NO: 52.

Substitution of Thr at position 2 of CDR1 (SEQ ID NO: 13) in the light chain variable region of SEQ ID NO: 52 with Ser (SEQ ID NO: 192). Thus, the present invention provides light chain variable regions in which CDR1 having the amino acid sequence of SEQ ID NO: 13 is substituted with CDR I having the amino acid sequence of SEQ ID NO: 192 in a light chain variable region having the amino acid sequence of SEQ ID NO: 52.

Substitution of Asn at position 1 of CDR2 (SEQ ID NO: 14) in the light chain variable region of SEQ ID NO: 52 with Asp (SEQ ID NO: 123). Thus, the present invention provides light chain variable regions in which CDR2 having the amino acid sequence of SEQ ID NO: 14 is substituted with CDR2 having the amino acid sequence of SEQ ID NO: 123 in a light chain variable region having the amino acid sequence of SEQ ID NO: 52.

Substitution of Lys at position 3 of CDR2 (SEQ ID NO: 14) in the light chain variable region of SEQ ID NO: 52 with Gln (SEQ ID NO: 124). Thus, the present invention provides light chain variable regions in which CDR2 having the amino acid sequence of SEQ ID NO: 14 is substituted with CDR2 having the amino acid sequence of SEQ ID NO: 124 in a light chain variable region having the amino acid sequence of SEQ ID NO: 52.

Substitution of Leu at position 5 of CDR2 (SEQ ID NO: 14) in the light chain variable region of SEQ ID NO: 52 with Glu (SEQ ID NO: 125). Thus, the present invention provides light chain variable regions in which CDR2 having the amino acid sequence of SEQ ID NO: 14 is substituted with CDR2 having the amino acid sequence of SEQ ID NO: 125 in a light chain variable region having the amino acid sequence of SEQ ID NO: 52.

Substitution of Lys at position 7 of CDR2 (SEQ ID NO: 14) in the light chain variable region of SEQ ID NO: 52 with Gln (SEQ ID NO: 126). Thus, the present invention provides light chain variable regions in which CDR2 having the amino acid sequence of SEQ ID NO: 14 is substituted with CDR2 having the amino acid sequence of SEQ ID NO: 126 in a light chain variable region having the amino acid sequence of SEQ ID NO: 52.

Substitution of Lys at position 7 of CDR2 (SEQ ID NO: 14) in the light chain variable region of SEQ ID NO: 52 with Asp (SEQ ID NO: 127). Thus, the present invention provides light chain variable regions in which CDR2 having the amino acid sequence of SEQ ID NO: 14 is substituted with CDR2 having the amino acid sequence of SEQ ID NO: 127 in a light chain variable region having the amino acid sequence of SEQ ID NO: 52.

Substitution of Arg at position 1 and Asn at position 5 of CDR1 (SEQ ID NO: 13) in the light chain variable region of SEQ ID NO: 52 with Gln and Asp, respectively (SEQ ID NO: 169). Thus, the present invention provides light chain variable regions in which CDR1 having the amino acid sequence of SEQ ID NO: 13 is substituted with CDR1 having the amino acid sequence of SEQ ID NO: 169 in a light chain variable region having the amino acid sequence of SEQ ID NO: 52.

Substitution of Lys at position 3, Leu at position 5, and Lys at position 7 of CDR2 (SEQ ID NO: 14) in the light chain variable region of SEQ ID NO: 52 with Gln, Glu, and Gln, respectively (SEQ ID NO: 170). Thus, the present invention provides light chain variable regions in which CDR2 having the amino acid sequence of SEQ ID NO: 14 is substituted with CDR2 having the amino acid sequence of SEQ ID NO: 170 in a light chain variable region having the amino acid sequence of SEQ ID NO: 52.

Substitution of Glu at position 5 of CDR3 (SEQ ID NO: 15) in the light chain variable region of SEQ ID NO: 52 with Asp (SEQ ID NO: 193). Thus, the present invention provides light chain variable regions in which CDR3 having the amino acid sequence of SEQ ID NO: 15 is substituted with CDR3 having the amino acid sequence of SEQ ID NO: 193 in a light chain variable region having the amino acid sequence of SEQ ID NO: 52.

Substitution of Ser at position 6 of CDR3 (SEQ ID NO: 15) in the light chain variable region of SEQ ID NO: 52 with Asp (SEQ ID NO: 194). Thus, the present invention provides light chain variable regions in which CDR3 having the amino acid sequence of SEQ ID NO: 15 is substituted with CDR3 having the amino acid sequence of SEQ ID NO: 194 in a light chain variable region having the amino acid sequence of SEQ ID NO: 52.

Substitution of Thr at position 9 of CDR3 (SEQ ID NO: 15) in the light chain variable region of SEQ ID NO: 52 with Phe (SEQ ID NO: 195). Thus, the present invention provides light chain variable regions in which CDR3 having the amino acid sequence of SEQ ID NO: 15 is substituted with CDR3 having the amino acid sequence of SEQ ID NO: 195 in a light chain variable region having the amino acid sequence of SEQ ID NO: 52.

In addition, the substitutions other than those described above include a substitution of Arg at position 3 of heavy chain FR2 having the amino acid sequence of SEQ ID NO: 97 with another amino acid. The amino acid after substitution is not particularly limited; but preferred examples thereof include Gln. When Arg at position 3 in SEQ ID NO: 97 has been replaced with Gln, Ala at position 5 may be substituted with Ser to produce a human FR2 sequence. The amino acid sequence in which Arg and Ala at positions 3 and 5 in the amino acid sequence of SEQ ID NO: 97 have been replaced with Gln and Ser, respectively, is shown in SEQ ID NO: 120. Thus, the present invention provides heavy chain variable regions in which FR2 having the amino acid sequence of SEQ ID NO: 97 is substituted with FR2 having the amino acid sequence of SEQ ID NO: 120 in a heavy chain variable region having the amino acid sequence of SEQ ID NO: 50 or 112.

Each of the above-described amino acid substitutions may be used alone or in combination with other amino acid substitutions described above. They also may be combined with amino acid substitutions other than those described above.

Specific examples of the antibodies in which the above-described substitutions have been carried out include, for example, antibodies that comprise a heavy chain variable region having the amino acid sequence of SEQ ID NO: 167, antibodies that comprise a light chain variable region having the amino acid sequence of SEQ ID NO: 168, and antibodies that comprise a heavy chain variable region having the amino acid sequence of SEQ ID NO: 167 and a light chain variable region having the amino acid sequence of SEQ ID NO: 168.

Furthermore, specific examples of the heavy chain variable regions in which the above-described substitutions have been carried out include, for example, the following heavy chain variable regions:
(1) heavy chain variable regions having the amino acid sequence of SEQ ID NO: 204 (H17);
(2) heavy chain variable regions having the amino acid sequence of SEQ ID NO: 205 (H19);
(3) heavy chain variable regions having the amino acid sequence of SEQ ID NO: 206 (H28);
(4) heavy chain variable regions having the amino acid sequence of SEQ ID NO: 207 (H30);
(5) heavy chain variable regions having the amino acid sequence of SEQ ID NO: 208 (H34);
(6) heavy chain variable regions having the amino acid sequence of SEQ ID NO: 209 (H42);
(7) heavy chain variable regions having the amino acid sequence of SEQ ID NO: 210 (H44);
(8) heavy chain variable regions having the amino acid sequence of SEQ ID NO: 211 (H46);
(9) heavy chain variable regions having the amino acid sequence of SEQ ID NO: 212 (H57);
(10) heavy chain variable regions having the amino acid sequence of SEQ ID NO: 213 (H71);
(11) heavy chain variable regions having the amino acid sequence of SEQ ID NO: 214 (H78);
(12) heavy chain variable regions having the amino acid sequence of SEQ ID NO: 215 (H92);
(13) heavy chain variable regions having the amino acid sequence of SEQ ID NO: 216 (H97); and
(14) heavy chain variable regions having the amino acid sequence of SEQ ID NO: 217 (H98).

Meanwhile, specific examples of the light chain variable regions in which the above-described substitutions carried out include, for example, the following light chain variable regions:
(15) light chain variable regions having the amino acid sequence of SEQ ID NO: 218 (L11);
(16) light chain variable regions having the amino acid sequence of SEQ ID NO: 219 (L12);
(17) light chain variable regions having the amino acid sequence of SEQ ID NO: 220 (L17); and
(18) light chain variable regions having the amino acid sequence of SEQ ID NO: 221 (L50).

Furthermore, specific examples of the antibodies comprising the above-described heavy chain and light chain variable regions include, for example, the following antibodies:
(19) antibodies that comprise the heavy chain variable region of (3) and the light chain variable region of (17) (H28L17);
(20) antibodies that comprise the heavy chain variable region of (4) and the light chain variable region of (17) (H30L17);
(21) antibodies that comprise the heavy chain variable region of (5) and the light chain variable region of (17) (H34L17);
(22) antibodies that comprise the heavy chain variable region of (6) and the light chain variable region of (17) (H42L17);
(23) antibodies that comprise the heavy chain variable region of (7) and the light chain variable region of (17) (H44L17);
(24) antibodies that comprise the heavy chain variable region of (8) and the light chain variable region of (17) (H46L17);
(25) antibodies that comprise the heavy chain variable region of (9) and the light chain variable region of (17) (H57L17);
(26) antibodies that comprise the heavy chain variable region of (10) and the light chain variable region of (17) (H71L17);
(27) antibodies that comprise the heavy chain variable region of (11) and the light chain variable region of (17) (H78L17);
(28) antibodies that comprise the heavy chain variable region of (12) and the light chain variable region of (17) (H92L17);
(29) antibodies that comprise the heavy chain variable region of (13) and the light chain variable region of (18) (H97L50); and
(30) antibodies that comprise the heavy chain variable region of (14) and the light chain variable region of (18) (H98L50).

The constant region used for the humanized antibodies of the present invention may be any constant region derived from a human antibody. Preferred examples of such constant regions derived from human antibodies include, for example, constant regions derived from IgG1 or IgG2. Moreover, constant regions in which one or more amino acids are substituted, deleted, added, and/or inserted in the constant region derived from a human antibody may also be used.

The constant regions in which one or more amino acids are substituted, deleted, added, and/or inserted in the constant region derived from a human antibody are not particularly limited, and include, for example, the following constant regions:

constant regions having the amino acid sequence of SEQ ID NO: 128 (M58);

constant regions having the amino acid sequence of SEQ ID NO: 129 (M14); and

constant regions having the amino acid sequence of SEQ ID NO: 62 (SKSC).

Specific examples of the heavy chains or antibodies having the above-described constant regions include, for example:
(1) heavy chains that comprise a variable region having the amino acid sequence of SEQ ID NO: 167 and a constant region having the amino acid sequence of SEQ ID NO: 128;
(2) heavy chains in which CDR2 having the amino acid sequence of SEQ ID NO: 171 in the heavy chains of (1) is substituted with CDR2 having the amino acid sequence of SEQ ID NO: 172;
(3) antibodies that comprise the heavy chain of (1) and a light chain having the amino acid sequence of SEQ ID NO: 152; and
(4) antibodies that comprise the heavy chain of (2) and a light chain having the amino acid sequence of SEQ ID NO: 152.

More specific examples of the humanized anti-NR10 antibodies of the present invention include, for example, the following antibodies:
(k) antibodies that comprise a heavy chain having the amino acid sequence of SEQ ID NO: 54 (H1-VH+constant region);
(1) antibodies that comprise a heavy chain having the amino acid sequence of SEQ ID NO: 130 (H1-VH+constant region);
(m) antibodies that comprise a light chain having the amino acid sequence of SEQ ID NO: 56 (L0-VL+constant region);
   (n) antibodies that comprise a heavy chain having the amino acid sequence of SEQ ID NO: 54 (H0-VH+constant region) and a light chain having the amino acid sequence of SEQ ID NO: 56 (L0-VL+constant region); and
(o) antibodies that comprise a heavy chain having the amino acid sequence of SEQ ID NO: 130 (H1-VH+constant region) and a light chain having the amino acid sequence of SEQ ID NO: 56 (L0-VL+constant region).

The heavy chain having the amino acid sequence of SEQ ID NO: 54 (H0-VH+constant region) and the light chain having the amino acid sequence of SEQ ID NO: 56 (L0-VL+constant region) may have a substitution, deletion, addition, and/or insertion of one or more amino acids. The substitution, deletion, addition, and/or insertion of amino acids may be carried out in either or both of the variable and constant regions.

Thus, the present invention provides:
(p) antibodies that comprise a heavy chain having an amino acid sequence in which one or more amino acids are substituted, deleted, added, and/or inserted in the amino acid sequence of SEQ ID NO: 54 (H0-VH+constant region);
(q) antibodies that comprise a heavy chain having an amino acid sequence in which one or more amino acids are substituted, deleted, added, and/or inserted in the amino acid sequence of SEQ ID NO: 130 (H1-VH+constant region);
(r) antibodies that comprise a light chain having an amino acid sequence in which one or more amino acids are substituted, deleted, added, and/or inserted in the amino acid sequence of SEQ ID NO: 56 (L0-VL+constant region);
(s) antibodies that comprise a heavy chain having an amino acid sequence in which one or more amino acids are substituted, deleted, added, and/or inserted in the amino acid sequence of SEQ ID NO: 54 (H0-VH+constant region) and a light chain having an amino acid sequence in which one or more amino acids are substituted, deleted, added, and/or inserted in the amino acid sequence of SEQ ID NO: 56 (L0-VL+constant region); and
(t) antibodies that comprise a heavy chain having an amino acid sequence in which one or more amino acids are substituted, deleted, added, and/or inserted in the amino acid sequence of SEQ ID NO: 130 (H1-VH+constant region) and a light chain having an amino acid sequence in which one or more amino acids are substituted, deleted, added, and/or inserted in the amino acid sequence of SEQ ID NO: 56 (L0-VL+constant region).

Without particular limitation, the antibodies of any one of (p) to (t) preferably have an activity similar to that of the antibodies of any one of (k) to (o).

The substitution, deletion, addition, and/or insertion of amino acids are not particularly limited, but specific examples thereof include, for example, the above-described amino acid substitutions.

Furthermore, the nucleotide sequence encoding the amino acid sequence of the above-described humanized heavy chain variable region (SEQ ID NO: 50) is shown in SEQ ID NO: 49. The nucleotide sequence encoding the amino acid sequence of the humanized light chain variable region (SEQ ID NO: 52) is shown in SEQ ID NO: 51. The nucleotide sequence encoding the amino acid sequence of the humanized heavy chain (SEQ ID NO: 54) is shown in SEQ ID NO: 53. The nucleotide sequence encoding the amino acid sequence of the humanized light chain (SEQ ID NO: 56) is shown in SEQ ID NO: 55.

Moreover, the present invention provides antibodies that recognize the same epitope as recognized by the antibodies of any one of (a) to (t) above. The binding to the same epitope is as already described above.

Furthermore, the present invention provides the following antibodies:
(u) antibodies that comprise a heavy chain having the amino acid sequence of SEQ ID NO: 151;
(v) antibodies that comprise a light chain comprising the amino acid sequence of SEQ ID NO: 152; and
(w) antibodies that comprise the heavy chain of (u) and the light chain of (v).

Moreover, the present invention provides the following heavy and light chains and antibodies:
(1) heavy chains having the amino acid sequence of SEQ ID NO: 222 (H17);
(2) heavy chains having the amino acid sequence of SEQ ID NO: 223 (H19);
(3) heavy chains having the amino acid sequence of SEQ ID NO: 224 (H28);
(4) heavy chains having the amino acid sequence of SEQ ID NO: 225 (H30);
(5) heavy chains having the amino acid sequence of SEQ ID NO: 226 (H34);
(6) heavy chains having the amino acid sequence of SEQ ID NO: 227 (H42);
(7) heavy chains having the amino acid sequence of SEQ ID NO: 228 (H44);
(8) heavy chains having the amino acid sequence of SEQ ID NO: 229 (H46);
(9) heavy chains having the amino acid sequence of SEQ ID NO: 230 (H57);
(10) heavy chains having the amino acid sequence of SEQ ID NO: 231 (H71);
(11) heavy chains having the amino acid sequence of SEQ ID NO: 232 (H78);
(12) heavy chains having the amino acid sequence of SEQ ID NO: 233 (H92);
(13) heavy chains having the amino acid sequence of SEQ ID NO: 234 (H97);
(14) heavy chains having the amino acid sequence of SEQ ID NO: 235 (H98);
(15) light chains having the amino acid sequence of SEQ ID NO: 236 (L11)
(16) light chains having the amino acid sequence of SEQ ID NO: 237 (L12);
(17) light chains having the amino acid sequence of SEQ ID NO: 238 (L17);
(18) light chains having the amino acid sequence of SEQ ID NO: 239 (L50);
(19) antibodies that comprise the heavy chain of (3) and the light chain of (17) (H28L17);
(20) antibodies that comprise the heavy chain of (4) and the light chain of (17) (H30L17);
(21) antibodies that comprise the heavy chain of (5) and the light chain of (17) (H34L17);
(22) antibodies that comprise the heavy chain of (6) and the light chain of (17) (H42L17);
(23) antibodies that comprise the heavy chain of (7) and the light chain of (17) (H44L17);
(24) antibodies that comprise the heavy chain of (8) and the light chain of (17) (H46L17);
(25) antibodies that comprise the heavy chain of (9) and the light chain of (17) (H57L17);
(26) antibodies that comprise the heavy chain of (10) and the light chain of (17) (H71L17);
(27) antibodies that comprise the heavy chain of (11) and the light chain of (17) (H78L17);
(28) antibodies that comprise the heavy chain of (12) and the light chain of (17) (H92L17);
(29) antibodies that comprise the heavy chain of (13) and the light chain of (18) (H97L50);
(30) antibodies that comprise the heavy chain of (14) and the light chain of (18) (H98L50);
(31) heavy chains having an amino acid sequence in which one or more amino acids are substituted, deleted, added and/or inserted in the heavy chains of any one of (1) to (14);
(32) light chains having an amino acid sequence in which one or more amino acids are substituted, deleted, added and/or inserted in the light chains of any one of (15) to (18);
(33) antibodies having an amino acid sequence in which one or more amino acids are substituted, deleted, added and/or inserted in the antibodies of any one of (19) to (30); and
(34) antibodies that recognize the same epitope as recognized by the antibodies of any one of (19) to (33).

The substitution, deletion, addition, and/or insertion of amino acids are as described above. Antibodies that recognize the same epitope as recognized by an antibody are also described above.

The present invention also provides genes encoding the variable regions, heavy chains, light chains, or antibodies of the present invention.

The present invention also provides vectors carrying the above-described genes. The present invention also provides host cells transformed with the above-described vectors.

The present invention also relates to methods for producing variable regions, heavy chains, light chains, or antibodies of the present invention, which comprise the step of culturing the above-described host cells.

The vectors, host cells, and culture of host cells are described herein below.

Antibodies that Recognize Domains

Preferred embodiments of the anti-NR 10 antibody of the present invention include antibodies that recognize domain 1 or domain 2. In the present invention, domain I refers to the region of amino acids at positions 21 to 120 (LPAKP to LENIA) in the amino acid sequence of human NR10 of SEQ ID NO: 76, where the amino acid numbering is based on the sequence including the signal peptide. In addition, in the present invention, domain 2 refers to the region of amino acids at positions 121 to 227 (KTEPP to EEEAP) in the amino acid sequence of human NR10 of SEQ ID NO: 76, where the amino acid numbering is based on the sequence including the signal peptide.

Such antibodies are not particularly limited; however, in general, they have a neutralizing activity, and preferably are humanized antibodies.

Examples of the preferred antibodies in the present invention include antibodies that recognize domain 1. The antibodies that recognize domain I have a strong neutralizing activity, and thus are particularly useful as pharmaceuticals.

### Antibodies (Neutralizing Activity)

The present invention also provides anti-NR10 antibodies having a neutralizing activity. In the present invention, the neutralizing activity against NR10 refers to an activity of inhibiting the binding between NR10 and its ligand IL-31, and preferably an activity of suppressing a biological activity based on NR10.

Antibodies having a NR10-neutralizing activity can be selected, for example, by adding candidate antibodies to an IL-31-dependent cell line and observing their growth-suppressing effect on the cell line. In this method, antibodies that suppress the growth of the IL-31-dependent cell line are determined as antibodies having a neutralizing activity against NR10.

### Antibodies (General)

The antibodies of the present invention are not limited in terms of their origin, and may be derived from any animals such as humans, mice, and rats. Moreover, the antibodies may be recombinant antibodies such as chimeric antibodies and humanized antibodies. As described above, the preferred antibodies of the present invention include humanized antibodies.

The chimeric antibodies contain, for example, the heavy and light chain constant regions of a human antibody, and the heavy and light chain variable regions of an antibody of a non-human mammal, such as mouse. The chimeric antibodies can be produced by known methods. For example, the antibodies can be produced by cloning an antibody gene from hybridomas, inserting it into an appropriate vector, and introducing the construct into hosts (sec, for example, Carl, A. K. Borrebaeck, James, W. Larrick, THERAPEUTIC MONOCLONAL ANTIBODIES, Published in the United Kingdom by MACMILLAN PUBLISHERS LTD, 1990). Specifically, cDNAs of the antibody variable regions (V regions) are synthesized from mRNA of hybridomas using reverse transcriptase. Once DNAs encoding the V regions of an antibody of interest are obtained, these are linked with DNAs encoding the constant regions (C regions) of a desired human antibody. The resulting constructs are inserted into expression vectors. Alternatively, the DNAs encoding the antibody V regions may be inserted into expression vectors comprising DNAs encoding the C regions of a human antibody. The DNAs are inserted into expression vectors so that they are expressed under the regulation of the expression regulatory regions, for example, enhancers and promoters. In the next step, host cells can be transformed with the expression vectors to allow expression of chimeric antibodies.

Methods for obtaining human antibodies are also known. For example, desired human antibodies with antigen-binding activity can be obtained by (1) sensitizing human lymphocytes with antigens of interest or cells expressing antigens of interest in vitro; and (2) fusing the sensitized lymphocytes with human myeloma cells such as U266 (see Japanese Patent Application Kokoku Publication No. (JP-B) H01-59878 (examined, approved Japanese patent application published for opposition)). Alternatively, the desired human antibody can also be obtained by immunizing a transgenic animal having an entire repertoire of human antibody genes with a desired antigen (see International Patent Application Publication Nos. WO 93/12227, WO 92/03918, WO 94/02602, WO 94/25585, WO 96/34096, and WO 96/33735).

Furthermore, techniques to obtain human antibodies by panning with a human antibody phage library are known. For example, the variable region of a human antibody is expressed as a single chain antibody (scFv) on the surface of a phage, using a phage display method, and phages that bind to the antigen can be selected. By analyzing the genes of selected phages, the DNA sequences encoding the variable regions of human antibodies that bind to the antigen can be determined. If the DNA sequences of scFvs that bind to the antigen are identified, appropriate expression vectors comprising these sequences can be constructed to obtain human antibodies. Such methods are well known. Reference can be made to WO 92/01047, WO 92/20791, WO 93/06213, WO 93/11236, WO 93/19172, WO 95/01438, WO 95/15388, and such.

The antibodies of the present invention include not only divalent antibodies as represented by IgG, but also monovalent antibodies, multivalent antibodies as represented by IgM, and bispecific antibodies capable of binding to different antigens, as long as they have a NR 10-binding activity and/or neutralizing activity. The multivalent antibodies of the present invention include multivalent antibodies in which the antigen-binding sites are all identical, and multivalent antibodies in which all or some of the antigen-binding sites are different. The antibodies of the present invention are not limited to full-length antibody molecules, but may also be low-molecular-weight antibodies or modified products thereof, as long as they bind to NR 10 protein.

Alternatively, the antibodies of the present invention may be low-molecular-weight antibodies. Such low-molecular-weight antibodies are antibodies including antibody fragments lacking some portions of a whole antibody (for example, whole IgG), and are not particularly limited as long as they retain NR10-binding and/or neutralizing activity. In the present invention, the low-molecular-weight antibodies are not particularly limited, as long as they contain a portion of whole antibodies. The low-molecular-weight antibodies preferably contain a heavy chain variable region (VH) or light chain variable region (VL). Particularly preferred low-molecular-weight antibodies contain both VH and VL. In addition, preferred examples of the low-molecular-weight antibodies of the present invention include low-molecular-weight antibodies containing CDRs of an antibody. The CDRs contained in the low-molecular-weight antibodies may include some or all of the six CDRs of an antibody.

The low-molecular-weight antibodies of the present invention preferably have a smaller molecular weight than whole antibodies. However, the low-molecular-weight antibodies may form multimers, for example, dimers, trimers, or tetramers, and thus their molecular weights can be greater than those of whole antibodies.

Specific examples of the antibody fragments include, for example, Fab, Fab′, F(ab′)2, and Fv. Meanwhile, specific examples of the low-molecular-weight antibodies include, for example, Fab, Fab′, F(ab′)2, Fv, scFv (single chain Fv), diabodies, and sc(Fv)2 (single chain (Fv)2). Multimers (for example, dimers, trimers, tetramers, and polymers) of these antibodies are also included in the low-molecular-weight antibodies of the present invention. Antibody fragments can be obtained, for example, by treating antibodies with enzymes to produce antibody fragments. Enzymes known to generate antibody fragments include, for example, papain, pepsin, and plasmin. Alternatively, a gene encoding such an antibody fragment can be constructed, introduced into an expression vector, and expressed in appropriate host cells (see, for example, Co, M. S. et al., J. Immunol. (1994)152, 2968-2976; Better, M. & Horwitz, A. H. Methods in Enzymology (1989)178, 476-496; Plueckthun, A. & Skerra, A. Methods in Enzymology (1989)178, 476-496; Lamoyi, E., Methods in Enzymology (1989)121, 652-663; Rousseaux, J. et al., Methods in Enzymology(l989)121, 663-669; Bird, R. E. et al., TIBTECH (1991)9, 132-137).

Digestive enzymes cleave a specific site of an antibody fragment, yielding antibody fragments of specific structures shown below. Genetic engineering techniques can be applied to such enzymatically-obtained antibody fragments to delete an arbitrary portion of the antibody.

Antibody fragments obtained by using the above-described digestive enzymes are as follows:
Papain digestion: F(ab)2 or Fab
Pepsin digestion: F(ab′)2 or Fab′
Plasmin digestion: Facb

The low-molecular-weight antibodies of the present invention include antibody fragments lacking an arbitrary region, as long as they have a NR10-binding activity and/or neutralizing activity.

“Diabody” refers to a bivalent antibody fragment constructed by gene fusion (Holliger P et al., Proc. Natl. Acad. Sci. USA 90: 6444-6448 (1993); EP 404,097; WO 93/11161, etc). Diabodies are dimers composed of two polypeptide chains. In each of the polypeptide chains forming a dimer, a VL and a VH are usually linked by a linker in the same chain. In general, the linker in a diabody is short enough such that the VL and VH cannot bind to each other. Specifically, the number of amino acid residues constituting the linker is, for example, about five residues. Thus, the VL and VH encoded on the same polypeptide cannot form a single-chain variable region fragment, and will form a dimer with another single-chain variable region fragment. As a result, the diabody has two antigen binding sites.

ScFv antibodies are single-chain polypeptides produced by linking a heavy chain variable region ([VH]) and a light chain variable region ([VL]) via a linker or such (Huston, J. S. et al., Proc. Natl. Acad. Sci. U.S.A. (1988) 85, 5879-5883; Pluckthun “The Pharmacology of Monoclonal Antibodies” Vol. 113, eds., Resenburg and Moore, Springer Verlag, New York, pp. 269-315, (1994)). The H-chain V region and L-chain V region of scFv may be derived from any antibody described herein. The peptide linker for linking the V regions is not particularly limited. For example, an arbitrary single-chain peptide containing about three to 25 residues can be used as the linker. Specifically, it is possible to use the peptide linkers or such described below.

The V regions of both chains can be linked, for example, by PCR as described above. First, among the following DNAs, a DNA encoding a complete or desired partial amino acid sequence is used as a template to link the V regions by PCR:
DNA sequence encoding an H chain or H-chain V region of an antibody, and
DNA sequence encoding an L chain or L-chain V region of an antibody.

DNAs encoding the V regions of H chain and L chain are amplified by PCR using a pair of primers having sequences corresponding to those at both ends of the DNA to be amplified. Then, a DNA encoding the peptide linker portion is prepared. The peptide linker-encoding DNA can also be synthesized by PCR. Here, nucleotide sequences that can be ligated to the amplification products of V regions synthesized separately are added to the 5′ end of the primers to be used. Then, PCR is carried out using each DNA of the [H chain V region DNA]-[peptide linker DNA]-[L chain V region DNA], and assembly PCR primers.

The assembly PCR primers are composed of a combination of a primer that anneals to the 5′ end of the [H chain V region DNA] and a primer that anneals to the 3′ end of the [L chain V region DNA]. In other words, the assembly PCR primers are a set of primers that can be used to amplify DNA encoding the full-length sequence of scFv to be synthesized. Meanwhile, nucleotide sequences that can be ligated to the V-region DNAs have been added to the [peptide linker DNA]. Thus, these DNAs are linked together, and then the whole scFv is ultimately generated as an amplification product by the assembly PCR primers. Once the scFv-encoding DNAs are generated, expression vectors carrying these DNAs and recombinant cells transformed with these expression vectors can be obtained by conventional methods. Furthermore, the scFv can be obtained by culturing the resulting recombinant cells to express the scFv-encoding DNAs.

The order of the heavy chain and light chain variable regions to be linked together is not particularly limited, and they may be arranged in any order. Examples of the arrangement are listed below.
[VH] linker [VL]
[VL] linker [VH]

sc(Fv)2 is a single-chain low-molecular-weight antibody produced by linking two VHs and two VLs using linkers and such (Hudson et al., J Immunol. Methods 1999; 231: 177-189). For example, sc(Fv)2 can be produced by linking scFvs via a linker.

Antibodies in which two VHs and two VLs are arranged in the order of VH-VL-VH-VL ([VH] linker [VL] linker [VH] linker [VL]) from the N terminus of the single-chain polypeptide are preferred. However, the order of the two VHs and two VLs is not limited to the above arrangement, and they may be arranged in any order. Examples of the arrangement are listed below:
[VL] linker [VH] linker [VH] linker [VL]
[VH] linker [VL] linker [VL] linker [VH]
[VH] linker [VH] linker [VL] linker [VL]
[VL] linker [VL] linker [VH] linker [VH]
[VL] linker [VH] linker [VL] linker [VH]

The amino acid sequence of the heavy chain variable region or light chain variable region in a low-molecular-weight antibody may contain a substitution, deletion, addition, and/or insertion. Furthermore, the heavy chain variable region and light chain variable region may also lack some portions or be added with other polypeptides, as long as they have antigen binding ability when linked together. Alternatively, the variable regions may be chimerized or humanized.

In the present invention, linkers which bind the variable regions of the antibody include arbitrary peptide linkers that can be introduced using genetic engineering, or synthetic linkers such as those disclosed in Protein Engineering, 9(3), 299-305, 1996.

The preferred linkers in the present invention are peptide linkers. The lengths of the peptide linkers are not particularly limited and those skilled in the art can appropriately select the lengths depending on the purpose. Typical lengths are one to 100 amino acids, preferably 3 to 50 amino acids, more preferably 5 to 30 amino acids, and particularly preferably 12 to 18 amino acids (for example, 15 amino acids).

Amino acid sequences of such peptide linkers include, for example:

Ser;
Gly-Ser;
Gly-Gly-Ser;
Ser-Gly-Gly;
(SEQ ID NO: 82)
Gly-Gly-Gly-Ser;
(SEQ ID NO: 83)
Ser-Gly-Gly-Gly;
(SEQ ID NO: 84)
Gly-Gly-Gly-Gly-Ser;
(SEQ ID NO: 85)
Ser-Gly-Gly-Gly-Gly;
(SEQ ID NO: 86)
Gly-Gly-Gly-Gly-Gly-Ser;
(SEQ ID NO: 87)
Ser-Gly-Gly-Gly-Gly-Gly;
(SEQ ID NO: 88)
Gly-Gly-Gly-Gly-Gly-Gly-Ser;
(SEQ ID NO: 89)
Ser-Gly-Gly-Gly-Gly-Gly-Gly;
(Gly-Gly-Gly-Gly-Ser (SEQ ID NO: 84))n;
and
(Ser-Gly-Gly-Gly-Gly (SEQ ID NO: 85))n,

where n is an integer of 1 or larger.

The amino acid sequence of peptide linker can be appropriately selected by those skilled in the art depending on the purpose. For example, the above-mentioned “n”, which determines the length of the peptide linker, is usually 1 to 5, preferably 1 to 3, and more preferably 1 or 2.

Synthetic linkers (chemical crosslinking agents) include crosslinking agents that are routinely used to crosslink peptides, for example, N-hydroxy succinimide (NHS), disuccinimidyl suberate (DSS), bis(sulfosuccinimidyl) suberate (BS3), dithiobis(succinimidyl propionate) (DSP), dithiobis(sulfosuccinimidyl propionate) (DTSSP), ethylene glycol bis(succinimidyl succinate) (EGS), ethylene glycol bis(sulfosuccinimidyl succinate) (sulfo-EGS), disuccinimidyl tartrate (DST), disulfosuccinimidyl tartrate (sulfo-DST), bis[2-(succinimidoxycarbonyloxy)ethyl] sulfone (BSOCOES), and bis[2-(sulfosuccinimidoxycarbonyloxy)ethyl] sulfone (sulfo-BSOCOES). These crosslinking agents are commercially available.

When four antibody variable regions are linked, three linkers are usually required. Such multiple linkers may be the same or different.

The antibodies of the present invention include antibodies in which one or more amino acid residues have been added to the amino acid sequence of an antibody of the present invention. Further, fusion proteins which result from a fusion between one of the above antibodies and a second peptide or protein is included in the present invention. The fusion proteins can be prepared by ligating a polynucleotide encoding an antibody of the present invention and a polynucleotide encoding a second peptide or polypeptide in frame, inserting this into an expression vector, and expressing the fusion construct in a host. Some techniques known to those skilled in the art are available for this purpose. The partner peptide or polypeptide to be fused with an antibody of the present invention may be a known peptide, for example, FLAG (Hopp, T. P. et al., BioTechnology 6, 1204-1210 (1988)), 6x His consisting of six His (histidine) residues, 10x His, influenza hemagglutinin (HA), human c-myc fragment, VSV-GP fragment, p18HIV fragment, T7-tag, HSV-tag, E-tag, SV40 T antigen fragment, lck tag,-tubulin fragment, B-tag, Protein C fragment. Other partner polypeptides to be fused with the antibodies of the present invention include, for example, GST (glutathione-S-transferase), HA (influenza hemagglutinin), immunoglobulin constant region,-galactosidase, and MBP (maltose-binding protein). A polynucleotide encoding one of these commercially available peptides or polypeptides can be fused with a polynucleotide encoding an antibody of the present invention. The fusion polypeptide can be prepared by expressing the fusion construct.

Furthermore, the antibodies of the present invention may be conjugated antibodies which are linked to any of various molecules including polymeric substances such as polyethylene glycol (PEG) and hyaluronic acid, radioactive substances, fluorescent substances, luminescent substances, enzymes, and toxins. Such conjugated antibodies can be obtained by chemically modifying the obtained antibodies. Methods for modifying antibodies have been established in this field (for example, U.S. Pat. No. 5,057,313 and U.S. Pat. No. 5,156,840). The “antibodies” of the present invention also include such conjugated antibodies.

Furthermore, the antibodies used in the present invention may be bispecific antibodies. The bispecific antibody refers to an antibody that has variable regions recognizing different epitopes in the same antibody molecule. In the present invention, the bispecific antibodies may recognize different epitopes on an NR10 molecule, or recognize NR10 with one antigen-binding site and a different substance with the other antigen-binding site.

Methods for producing bispecific antibodies are known. Bispecific antibodies can be prepared, for example, by linking two antibodies that recognize different antigens. Antibodies to be linked together may be half molecules each of which contains an H chain and an L chain, or quarter molecules that consist of only one H chain. Alternatively, hybridomas producing different monoclonal antibodies can be fused to produce a bispecific antibody-producing fused cell. Furthermore, bispecific antibodies can be produced by genetic engineering techniques.

The antibodies of the present invention may differ in amino acid sequence, molecular weight, isoelectric point, presence/absence of sugar chains, and conformation depending on the cell or host producing the antibody or the purification method as described below. However, a resulting antibody is included in the present invention, as long as it is functionally equivalent to an antibody of the present invention. For example, when an antibody of the present invention is expressed in prokaryotic cells, for example E. coli, a methionine residue is added to the N terminus of the original antibody amino acid sequence. Such antibodies are included in the present invention.

### Antibody Production

The antibodies of the present invention may be polyclonal or monoclonal antibodies. Such monoclonal antibodies having NR10-binding and/or neutralizing activity can be obtained, for example, by the following procedure: anti-NR10 monoclonal antibodies are prepared by using as an antigen NR10 or a fragment thereof that is derived from a mammal such as human or mouse by known methods, and then antibodies having NR10-binding and/or neutralizing activity are selected from the thus obtained anti-NR10 monoclonal antibodies. Specifically, a desired antigen or cells expressing the desired antigen are used as a sensitizing antigen for immunization according to conventional immunization methods. Anti-NR10 monoclonal antibodies can be prepared by fusing the obtained immune cells with known parental cells using conventional cell fusion methods, and screening them for monoclonal antibody-producing cells (hybridomas) by conventional screening methods. Animals to be immunized include, for example, mammals such as mice, rats, rabbits, sheep, monkeys, goats, donkeys, cows, horses, and pigs. The antigen can be prepared using the known NR10 gene sequence according to known methods, for example, by methods using baculovirus (for example, WO 98/46777).

Hybridomas can be prepared, for example, according to the method of Milstein et al. (Kohler, G. and Milstein, C., Methods Enzymol. (1981) 73: 3-46) or such. When the immunogenicity of an antigen is low, immunization may be performed after linking the antigen with a macromolecule having immunogenicity, such as albumin.

Embodiments of the antibodies of the present invention that have a binding and/or neutralizing activity against NR10 include monoclonal antibodies that have a binding and/or neutralizing activity against human NR10. Antigens used to prepare monoclonal antibodies that have a binding and/or neutralizing activity against human NR10 are not particularly limited, as long as they enable preparation of antibodies that have a binding and/or neutralizing activity against human NR10. For example, it is known that there are a number of variants of human NR10, and any variant may be used as an immunogen as long as it enables preparation of antibodies that have a binding and/or neutralizing activity against human NR10. Alternatively, under the same condition, a peptide fragment of NR10 or a protein in which artificial mutations have been introduced into the natural NR10 sequence may be used as an immunogen. Human NR10.3 is one of preferred immunogens in preparing antibodies that have an activity of binding and/or neutralizing NR10 in the present invention.

Furthermore, the binding and/or neutralizing activity of antibody against NR10 can be measured, for example, by observing the effect of suppressing the growth of the IL-31-dependent cell line as described in the Examples.

Meanwhile, monoclonal antibodies can also be obtained by DNA immunization. DNA immunization is a method in which a vector DNA constructed such that the gene encoding an antigen protein can be expressed in an animal to be immunized is administered to the animal, and the immunogen is expressed within the body of the animal to provide immunostimulation. As compared to common immunization methods based on the administration of protein antigens, the DNA immunization is expected to be advantageous in that:
it enables immunostimulation while retaining the structure of a membrane protein; and
the immunogen does not need to be purified.

On the other hand, it is difficult to combine DNA immunization with an immunostimulating means such as an adjuvant.

In order to obtain a monoclonal antibody by DNA immunization, first, DNA encoding NR 10 is administered to an animal to be immunized. The DNA encoding NR10 can be synthesized by known methods such as PCR. The resulting DNA is inserted into an appropriate expression vector, and administered to the animal to be immunized. Expression vectors that can be used include commercially available expression vectors such as pcDNA3.1. The vector can be administered to the living body by conventional methods. For example, DNA immunization can be carried out by introducing gold particles coated with the expression vector into cells by gene gun. Booster using NR10-expressing cells after DNA immunization is a preferred method to yield a monoclonal antibody.

Once the mammal is immunized as described above and the serum level of a desired antibody is confirmed to be increased, immune cells are collected from the mammal and subjected to cell fusion. Preferred immune cells are spleen cells in particular.

Mammalian myeloma cells are used for fusion with the above immune cells. It is preferred that myeloma cells have appropriate selection markers for screening. The selection marker refers to a phenotype that allows (or does not allow) survival under particular culture conditions. Known selection markers include hypoxanthine-guanine phosphoribosyltransferase deficiency (hereinafter abbreviated as “HGPRT deficiency”) and thymidine kinase deficiency (hereinafter abbreviated as “TK deficiency”). HGPRT-or TK-deficient cells exhibit hypoxanthine-aminopterin-thymidine sensitivity (hereinafter abbreviated as “HAT sensitivity”). In HAT selection medium, HAT-sensitive cells cannot synthesize DNA and thus will dic. However, when fused with normal cells, they can continue to synthesize DNA via the salvage pathway of the normal cells and thus can grow even in HAT selection medium.

HGPRT-or TK-deficient cells can be selected using a medium containing 6-thioguanine, 8-azaguanine (hereinafter abbreviated as “8AG”), or 5′-bromodeoxyuridinc. While normal cells are killed due to incorporation of these pyrimidine analogs into DNA, cells lacking these enzymes can survive in the selection medium because they cannot incorporate these pyrimidine analogs. Another selection marker called G418 resistance confers resistance to 2-deoxystreptamine antibiotics (gentamicin analogs) due to the neomycin resistance gene. Various myeloma cells suitable for cell fusion are known.

Cell fusion between immune cells and myeloma cells can be essentially carried out according to known methods, for example, the method by Kohler and Milstein (Kohler. G. and Milstein, C., Methods Enzymol. (1981) 73, 3-46).

More specifically, cell fusion can be carried out, for example, in a common culture medium in the presence of a cell fusion-promoting agent. The fusion-promoting agent includes, for example, polyethylene glycol (PEG) and Sendai virus (HVJ). If required, an auxiliary agent such as dimethyl sulfoxide may also be added to improve fusion efficiency.

The immune cells and myeloma cells may be used at an arbitrarily determined ratio. For example, the ratio of immune cells to myeloma cells is preferably from 1 to 10. Culture media to be used for cell fusion include, for example, media that are suitable for the cell growth of myeloma cell line, such as RPMI 1640 and MEM, and other common culture media used for this type of cell culture. In addition, the culture media may also be supplemented with serum supplement such as fetal calf serum (FCS).

Predetermined amounts of immune cells and myeloma cells are mixed well in the culture medium, and then mixed with a PEG solution pre-heated to 37° C. to produce fused cells (hybridomas). In the cell fusion method, for example, PEG with mean molecular weight of about 1,000-6,000 can be added to the cells typically at a concentration of 30% to 60% (w/v).

Then, successive addition of the appropriate culture medium listed above and removal of supernatant by centrifugation are repeated to eliminate the cell fusion agent and such, which are unfavorable to the growth of hybridomas.

The resulting hybridomas can be screened using a selection medium according to the selection marker possessed by myeloma cells used in the cell fusion. For example, HGPRT-or TK-deficient cells can be screened by culturing them in a HAT medium (a medium containing hypoxanthinc, aminopterin, and thymidine). Specifically, when HAT-sensitive myeloma cells are used in cell fusion, cells successfully fused with normal cells can be selectively grown in the HAT medium. The cell culture using the above HAT medium is continued for a sufficient period of time to allow all cells except the desired hybridomas (non-fused cells) to dic.

Specifically, in general, the desired hybridomas can be selected by culturing the cells for several days to several weeks. Then, screening and single cloning of hybridomas that produce an antibody of interest can be carried out by performing ordinary limiting dilution methods. Alternatively, antibodies that recognize NR 10 can be prepared by the method described in WO 03/104453.

Screening and single cloning of an antibody of interest can be suitably carried out by known screening methods based on antigen-antibody reaction. For example, an antigen is bound to a carrier such as beads made of polystyrene or such and commercially available 96-well microtiter plates, and then reacted with the culture supernatant of hybridoma. Next, the carrier is washed and then reacted with an enzyme-labeled secondary antibody or such. When the culture supernatant contains an antibody of interest reactive to the sensitizing antigen, the secondary antibody binds to the carrier via this antibody. Finally, the secondary antibody bound to the carrier is detected to determine whether the culture supernatant contains the antibody of interest. Hybridomas producing a desired antibody capable of binding to the antigen can be cloned by the limiting dilution method or such. Not only the antigen used for immunization but also an NR10 protein substantially equivalent thereto can be preferably used as an antigen for this purpose. For example, a cell line expressing NR10, the extracellular domain of NR10, or an oligopeptide composed of a partial amino acid sequence constituting the domain may be used as the antigen.

In addition to the above-described method for preparing hybridomas through immunization of a nonhuman animal with an antigen, antibodies of interest can also be obtained by sensitizing human lymphocytes with an antigen. Specifically, first, human lymphocytes are sensitized with an NR10 protein in vitro. Then, the sensitized lymphocytes are fused with an appropriate fusion partner. For example, human-derived myeloma cells with the ability to divide permanently can be used as the fusion partner (see Japanese Patent Application Kokoku Publication No. (JP-B) H1-59878 (examined, approved Japanese patent application published for opposition). Antibodies obtained by this method are human antibodies having an activity of binding to the NR10 protein.

The nucleotide sequence encoding an anti-NR10 antibody obtained by the above-described method or such, and its amino acid sequence can be obtained by methods known to those skilled in the art.

Based on the obtained sequence of the anti-NR10 antibody, the anti-NR10 antibody can be prepared, for example, by genetic recombination techniques known to those skilled in the art. Specifically, a polynucleotide encoding an antibody can be constructed based on the sequence of the NR 10-recognizing antibody, inserted into an expression vector, and then expressed in appropriate host cells (see for example, Co, M. S. et al., J. Immunol. (1994) 152, 2968-2976; Better, M. and Horwitz, A. H., Methods Enzymol. (1989) 178, 476-496; Pluckthun, A. and Skerra, A., Methods Enzymol. (1989) 178, 497-515; Lamoyi, E., Methods Enzymol. (1986) 121, 652-663; Rousseaux, J. et al., Methods Enzymol. (1986) 121, 663-669; Bird, R. E. and Walker, B. W., Trends Biotechnol. (1991) 9, 132-137).

The vectors include M13 vectors, pUC vectors, pBR322, pBluescript, and pCR-Script. Alternatively, when aiming to subclone and excise cDNA, the vectors include, for example, pGEM-T, pDIRECT, and pT7, in addition to the vectors described above. Expression vectors are particularly useful when using vectors for producing the antibodies of the present invention. For example, when aiming for expression in E. coli such as JM109, DH5 , HB101, and XL1-Blue, the expression vectors not only have the above-described characteristics that allow vector amplification in E. coli, but must also carry a promoter that allows efficient expression in E. coli, for example, lacZ promoter (Ward et al., Nature (1989) 341, 544-546; FASEB J. (1992) 6, 2422-2427), araB promoter (Better et al., Science (1988) 240, 1041-1043), T7 promoter or such. Such vectors include pGEX-5X-1 (Pharmacia), “QIAexpress system” (Qiagen), pEGFP, or pET (in this case, the host is preferably BL21 that expresses T7 RNA polymerase) in addition to the vectors described above.

The vectors may contain signal sequences for antibody secretion. As a signal sequence for antibody secretion, a pelB signal sequence (Lei, S. P. et al J. Bacteriol. (1987) 169, 4379) may be used when a protein is secreted into the E. coli periplasm. The vector can be introduced into host cells by calcium chloride or electroporation methods, for example.

In addition to vectors for E. coli, the vectors for producing the antibodies of the present invention include mammalian expression vectors (for example, pcDNA3 (Invitrogen), pEF-BOS (Nucleic Acids. Res. 1990, 18(17), p5322), pEF, and pCDM8), insect cell-derived expression vectors (for example, the “Bac-to-BAC baculovirus expression system” (Gibco-BRL) and pBacPAK8), plant-derived expression vectors (for example, pMHI and pMH2), animal virus-derived expression vectors (for example, pHSV, pMV, and pAdexLcw), retroviral expression vectors (for example, pZIPneo), yeast expression vectors (for example, “Pichia Expression Kit” (Invitrogen), pNV11, and SP-Q01), and Bacillus subtilis expression vectors (for example, pPL608 and pKTH50), for example.

When aiming for expression in animal cells such as CHO, COS, and NIH3T3 cells, the vectors must have a promoter essential for expression in cells, for example, SV40 promoter (Mulligan et al., Nature (1979) 277, 108), MMLV-LTR promoter, EFI promoter (Mizushima et al., Nucleic Acids Res. (1990) 18, 5322), and CMV promoter, and more preferably they have a gene for selecting transformed cells (for example, a drug resistance gene that allows evaluation using an agent (neomycin, G418, or such). Vectors with such characteristics include pMAM, pDR2, pBK-RSV, pBK-CMV, POPRSV, and pOP13, for example.

In addition, the following method can be used for stable gene expression and gene amplification in cells: CHO cells deficient in a nucleic acid synthesis pathway are introduced with a vector (for example, pSV2-dhfr (Molecular Cloning 2nd edition, Cold Spring Harbor Laboratory Press, 1989)) that carries a DHFR gene which compensates for the deficiency, and the vector is amplified using methotrexate (MTX). Alternatively, the following method can be used for transient gene expression: COS cells with a gene expressing SV40 T antigen on their chromosome are transformed with a vector (pcD and such) with an SV40 replication origin. Replication origins derived from polyoma virus, adenovirus, bovine papilloma virus (BPV), and such can also be used. To amplify gene copy number in host cells, the expression vectors may further carry selection markers such as aminoglycoside transferase (APH) gene, thymidinc kinase (TK) gene, E. coli xanthine-guanine phosphoribosyltransferase (Ecogpt) gene, and dihydrofolate reductase (dhfr) gene.

The antibodies of the present invention obtained by the methods described above can be isolated from inside host cells or from outside the cells (the medium, or such), and purified to homogeneity. The antibodies can be isolated and purified by methods routinely used for isolating and purifying antibodies, and the type of method is not limited. For example, the antibodies can be isolated and purified by appropriately selecting and combining column chromatography, filtration, ultrafiltration, salting out, solvent precipitation, solvent extraction, distillation, immunoprecipitation, SDS-polyacrylamide gel electrophoresis, isoclectrofocusing, dialysis, recrystallization, and such.

The chromatographies include, for example, affinity chromatography, ion exchange chromatography, hydrophobic chromatography, gel filtration, reverse phase chromatography, and adsorption chromatography (Strategies for Protein Purification and Characterization: A Laboratory Course Manual. Ed Daniel R. Marshak et al., Cold Spring Harbor Laboratory Press, 1996). The chromatographic methods described above can be conducted using liquid chromatography, for example, HPLC and FPLC. Columns that can be used for affinity chromatography include protein A columns and protein G columns. Columns using protein A include, for example, Hyper D, POROS, and Sepharose FF (GE Amersham Biosciences). The present invention includes antibodies that are highly purified using these purification methods.

The NR10-binding activity of the obtained antibodies can be determined by methods known to those skilled in the art. Methods for determining the antigen-binding activity of an antibody include, for example, ELISA (enzyme-linked immunosorbent assay), EIA (enzyme immunoassay), RIA (radioimmunoassay), and fluorescent antibody method. For example, when enzyme immunoassay is used, antibody-containing samples, such as purified antibodies and culture supernatants of antibody-producing cells, are added to antigen-coated plates. A secondary antibody labeled with an enzyme, such as alkaline phosphatase, is added and the plates are incubated. After washing, an enzyme substrate, such as p-nitrophenyl phosphate, is added, and the absorbance is measured to evaluate the antigen-binding activity.

### Pharmaceutical Compositions

The present invention also provides pharmaceutical compositions comprising the antibody mentioned above as an active ingredient. Moreover, the present invention provides therapeutic agents for inflammatory diseases which comprise the antibody mentioned above as an active ingredient.

In the present invention, inflammatory disease refers to diseases with pathological features involved in cytological and histological reactions that occur in affected blood vessels and adjacent tissues in response to an injury or abnormal stimulation caused by physical, chemical, or biological agents (Stedman's Medical Dictionary, 5th Ed., MEDICAL VIEW CO., 2005). Generally, inflammatory diseases include, dermatitis (atopic dermatitis, chronic dermatitis, and such), inflammatory bowel diseases (colitis and such), asthma, arthritis (rheumatoid arthritis, osteoarthritis, and such), bronchitis, Th2 autoimmune diseases, systemic lupus erythematosus, myasthenia gravis, chronic GVHD, Crohn's disease, spondylitis deformans, lumbar pain, gout, inflammation after surgery or injury, swelling, neuralgia, laryngopharyngitis, cystitis, hepatitis (non-alcoholic steatohepatitis, alcoholic hepatitis, and such), hepatitis B, hepatitis C, arteriosclerosis, and pruritus.

Preferred examples of inflammatory diseases that are subjects of the present invention include atopic dermatitis, chronic dermatitis, rheumatism, osteoarthritis, chronic asthma, and pruritus.

The phrase “comprise(s) an anti-NR10 antibody as an active ingredient” means comprising an anti-NR 10 antibody as at least one of the active ingredients, and does not limit the proportion of the antibody. In addition, the therapeutic agents for inflammatory diseases in the present invention may also comprise, in combination with the anti-NR10 antibody mentioned above, other ingredients that enhance the treatment of inflammatory diseases.

The therapeutic agents of the present invention may also be used for preventive purposes.

The anti-NR10 antibody of the present invention may be prepared as formulations according to standard methods (see, for example, Remington's Pharmaceutical Science, latest edition, Mark Publishing Company, Easton, USA). Further, they may contain pharmaceutically acceptable carriers and/or additives if necessary. For example, they may contain surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, and corrigents. However, without limitation to these, the agents for preventing or treating inflammatory diseases of the present invention may contain other commonly used carriers. Such carriers specifically include light anhydrous silicic acid, lactose, crystalline cellulose, mannitol, starch, carmelose calcium, carmelose sodium, hydroxypropylcellulose, hydroxypropylmethylcellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, and inorganic salt. The agents may also contain other low-molecular-weight polypeptides, proteins such as serum albumin, gelatin, and immunoglobulin, and amino acids such as glycine, glutamine, asparagine, arginine, and lysine. When the anti-NR10 antibody is prepared as an aqueous solution for injection, the anti-NR10 antibody may be dissolved in an isotonic solution containing, for example, physiological saline, dextrose, or other adjuvants. The adjuvants may include, for example, D-sorbitol, D-mannose, D-mannitol, and sodium chloride. In addition, appropriate solubilizing agents, for example, alcohols (for example, ethanol), polyalcohols (for example, propylene glycols and PEGs), and non-ionic detergents (polysorbate 80 and HCO-50) may be used concomitantly.

If necessary, anti-NR 10 antibodies may be encapsulated in microcapsules (microcapsules made of hydroxymethylcellulose, gelatin, polymethylmethacrylate, and the like), and made into components of colloidal drug delivery systems (liposomes, albumin microspheres, microemulsions, nano-particles, and nano-capsules) (for example, see “Remington's Pharmaceutical Science 16th edition” &, Oslo Ed. (1980)). Moreover, methods for making sustained-release drugs are known, and these can be applied for anti-NR10 antibodies (Langer et al., J. Biomed. Mater. Res. (1981) 15, 167-277; Langer, Chem. Tech. (1982) 12, 98-105; U.S. Pat. No. 3,773,919; European Patent Application (EP) No. 58,481; Sidman et al., Biopolymers (1983) 22, 547-56; EP 133,988).

The pharmaceutical compositions of the present invention can be administered either orally or parenterally, but are preferably administered parenterally. Specifically, the agents are administered to patients by injection or percutaneous administration. Injections include, for example, intravenous injections, intramuscular injections, and subcutaneous injections, for systemic or local administration. The agents may be given to sites where inflammation is to be suppressed, or areas surrounding the sites by local infusion, intramuscular injection in particular.

The administration methods can be properly selected according to the patient's age and condition. The single-administration dose can be selected, for example, from within the range of 0.0001 to 100 mg of the active ingredient per kg body weight. Alternatively, for example, when the agents are administered to human patients, the dose of the active ingredient can be selected from within the range of 0.001 to 1,000 mg/kg body weight. The single-administration dose preferably contains, for example, about 0.01 to 50 mg/kg body weight of the antibody of the present invention. However, the dose of an agent for preventing or treating inflammatory diseases of the present invention is not limited to these examples.

All prior-art documents cited in the present specification are herein incorporated by reference.

### EXAMPLES

Herein below, the present invention will be specifically described with reference to Examples, but it is not to be construed as being limited thereto.

### Example 1

### Preparation of Hybridomas

### 1.1. Preparation of Human and Cynomolgus Monkey NR 10 Plasmids for DNA Immunization

1.1.1. Preparation of Expression Vectors for hNR10 and cynNR10

Human NR10 (nucleotide sequence, SEQ ID NO: 75; amino acid sequence, SEQ ID NO: 76) was inserted into the expression vector pMacII, which expresses a protein under the control of mouse-actin promoter (WO2005/054467), to prepare an expression vector for hNR10. In the same manner, an expression vector for cynNR 10 was constructed from cynomolgus monkey NR10 (nucleotide sequence, SEQ ID NO: 65; amino acid sequence, SEQ ID NO: 66).

### 1.1.2. Preparation of DNA Cartridge

In order to use the hNR10 or cynNR 10 expression vector prepared in 1.1.1 for DNA immunization of mice, the Helios Gene Gun Cartridge Kit (BIO-RAD) was used to produce a DNA cartridge for each DNA that allows immunization with 1 g of DNA at one time.

### 1.2. Preparation of Hybridomas Producing Anti-Human NR10 Antibody

1.2.1. Preparation of Hybridomas using Mice Immunized with Human or Cynomolgus Monkey NR10

Ten Balb/c mice (female; six weeks old at the beginning of immunization; Charles River Laboratories Japan) were immunized with human or cynomolgus monkey NR10 by the following procedure. For primary immunization, the mice were immunized with the DNA cartridge prepared with the hNR 10 expression vector using the Helios Gene Gun System (BIO-RAD). One week later, secondary immunization was performed by the Helios Gene Gun System (BIO-RAD) using the DNA cartridge prepared with the cynNR10 expression vector. The third and subsequent immunizations were carried out at one-week intervals using the hNR10 and cynNR10 expression vectors alternately. After the titer of serum antibody against human NR10 was confirmed to be elevated, a human NR10 protein (extracellular domain) (Referential Example 4) diluted with PBS(−) was intravenously administered at 10 g/head as the final immunization. Four days after the final immunization, mouse spleen cells were fused with mouse myeloma P3X63Ag8U.1 cells (abbreviated as P3U1; ATCC CRL-1597) by a conventional method using PEG1500 (Roche Diagnostics). The resulting fused cells, i.e., hybridomas, were cultured in RPMI1640 supplemented with 10% FBS (hereinafter abbreviated as 10% FBS/RPMI1640).

### 1.2.2. Selection of Hybridomas

On the next day of fusion, the fused cells were suspended in a semisolid medium (StemCells), and cultured for selection as well as colonization of hybridomas.

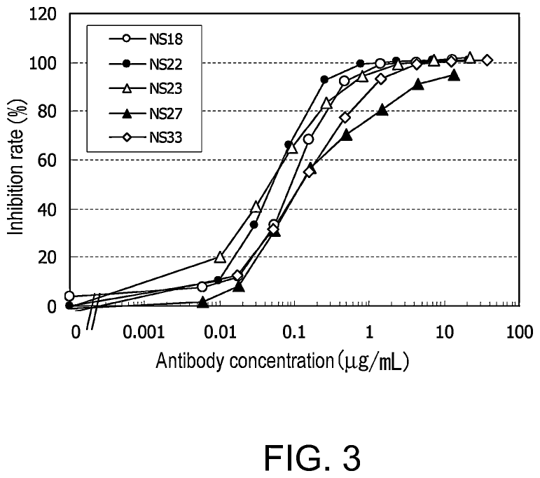

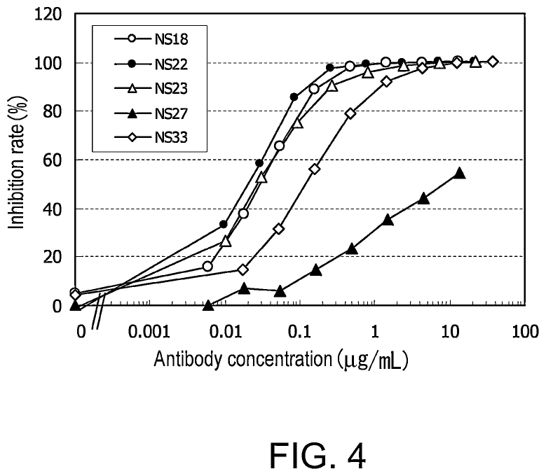

After nine or ten days of fusion, hybridoma colonies were picked up and each colony was seeded into each well of 96-well plates containing the HAT selection medium (10% FBS/RPMI1640, 2 vol % of HAT 50x concentrate (Dainippon Pharmaceutical), and 5 vol % of BM-Condimed HI (Roche Diagnostics)). After three to four days of culture, the culture supernatant was collected from each well to determine the concentration of mouse IgG in the supernatant. The culture supernatants in which mouse IgG was detected were assessed for a neutralizing activity using a human IL-31-dependent cell line (hNR10/hOSMR/BaF3 cells; Referential Example 2), and several clones having a strong NR10-neutralizing activity were obtained (FIG. 3). Clones that suppress the human IL-31-induced growth of cells in a concentration-dependent manner and suppress the cynomolgus monkey IL-31-induced growth of cells (cynNR10/cynOSMR/BaF3 cells; Referential Example 2) in a concentration-dependent manner were obtained (FIG. 4).

### Example 2

### Preparation of Chimeric Antibodies

### Preparation of Expression Vectors for Chimeric Antibodies

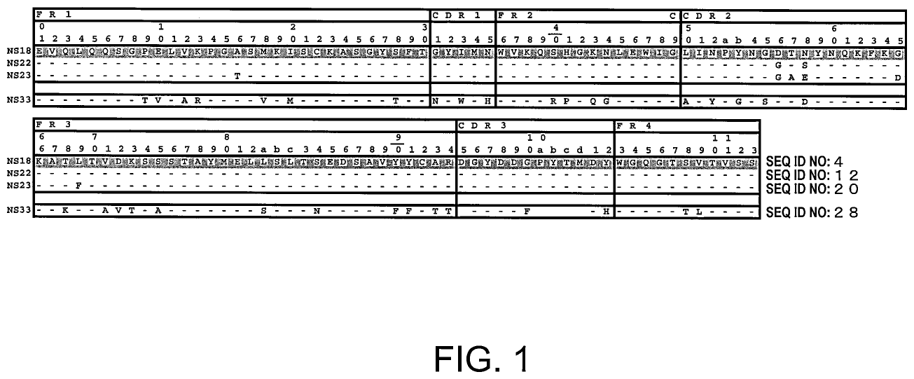

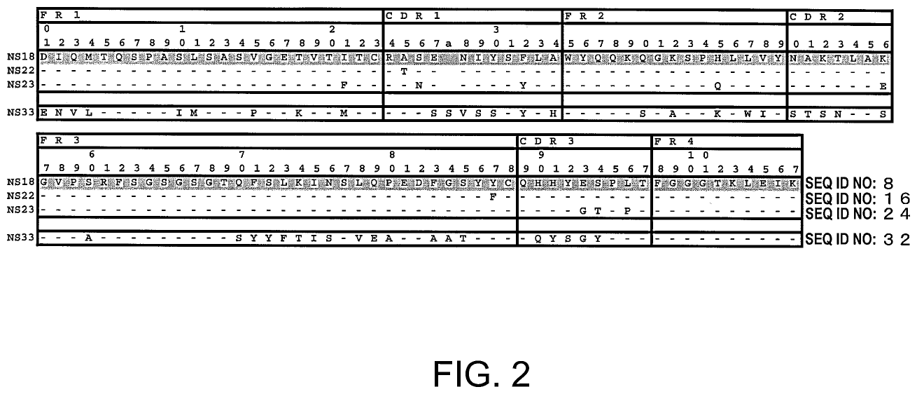

Total RNAs were extracted from the hybridomas using RNeasy Mini Kits (QIAGEN), and cDNAs were synthesized from them using SMART RACE cDNA Amplification Kit (BD Biosciences). Antibody variable region genes were isolated by PCR using PrimeSTAR HS DNA polymerase (TaKaRa), 10x Universal Primer A Mix attached to SMART RACE cDNA Amplification Kit (BD Biosciences), and primers designed for each antibody constant region (H chain, mlgG1-rnot; L chain, mlgK-rnot). The nucleotide sequence of each isolated DNA fragment was determined with ABI PRISM 3730xL DNA Sequencer or ABI PRISM 3700 DNA Sequencer (Applied Biosystems), using BigDye Terminator Cycle Sequencing Kit (Applied Biosystems) according to the method described in the appended instruction manual. The determined amino acid sequences of H chain and L chain variable regions in the mouse antibodies NS18, NS22, NS23, and NS33 were shown in FIGS. 1 and 2, respectively.

Each of the resulting H and L chain fragments was subjected to PCR using PrimeSTAR HS DNA Polymerase (TaKaRa) and the primer sets shown in Table 1. The resulting amplified fragments were ligated with the constant region (human 1 or 12, and human O, respectively), and then inserted into an animal cell expression vector. The nucleotide sequence of each DNA fragment was determined with ABI PRISM 3730xL DNA Sequencer or ABI PRISM 3700 DNA Sequencer (Applied Biosystems), using BigDye Terminator Cycle Sequencing Kit (Applied Biosystems) according to the method described in the appended instruction manual.

TABLE 1

| | Sequence (5′→3′) | SEQ ID NO: |
|---|---|---|
| mIgG1-rnot | TAATAGCGGCCGCTCATTATTTACCAGGAGAGTGGGAGAG | 90 |
| mIgK-rnot | TAATAGCGGCCGCTCATTAACACTCATTCCTGTTGAAGCT | 91 |
| mNS18H-feco | GACGAATTCCACCATGGGATGGAGCTGGATCTT | 92 |
| mNS18L-feco | GACGAATTCCACCATGAGTGTGCCCACTCAGGT | 93 |
| mNS33H-feco | GACGAATTCCACCATGGAATGTAACTGGATACT | 94 |
| mNS33L-feco | GACGAATTCCACCATGGATTTTCTGGTGCAGAT | 95 |

| | Forward primer | Reverse primer |
|---|---|---|
| NS18 H chain | mNS18H-feco | mIG1-rnot |
| NS18 L chain | mNS18L-feco | mIGK-rnot |
| NS22 H chain | Mns18H-feco | mIG1-rnot |
| NS22 L chain | mNS18L feco | mIGK-rnot |
| NS23 H chain | mNS18H-feco | mIG1-rnot |
| NS23 L chain | mNS18L feco | mIGK-rnot |
| NS33 H chain | mNS33H-feco | mIG1-rnot |
| NS33 L chain | mNS33L-feco | mIGK-rnot |

### Preparation of Chimeric Antibodies

Human embryonic kidney cancer cell line HEK293H (Invitrogen) was suspended in DMEM (Invitrogen) supplemented with 10% fetal bovine serum (Invitrogen), and 10 ml of cells were seeded into dishes for adherent cells (10 cm in diameter; CORNING) at a cell density of 6×10⁵ cells/ml. The cells were incubated in a CO₂ incubator (37°° C., 5% CO₂) for one whole day and night. Then, the medium was removed by aspiration, and 6.9 ml of CHO-S-SFMII medium (Invitrogen) was added. CHO-S-SFMII medium was added to the prepared plasmid DNA mixture (13.8 □g in total) to a volume of 700 □l. This was mixed with 20.7 □l of 1 □g/ml polyethyleneimine (Polysciences Inc.), and allowed to stand at room temperature for 10 minutes. The solution was added to the cells in each dish. The cells were incubated in a CO₂ incubator (37° C., 5% CO₂) for four to five hours. Then, 6.9 ml of CHO-S-SFMII medium (Invitrogen) was added, and the cells were incubated in a CO₂ incubator for three to four days. The culture supernatants were collected and then centrifuged (approx. 2000 g, five minutes, room temperature) to remove the cells. The supernatants were filtered through 0.22-m filter MILLEX®-GV (Millipore). Each sample was stored at 4° C. until use. Antibodies were purified from the supernatants using Protein G Sepharose (Amersham Biosciences). The purified antibodies were concentrated with Amicon Ultra 15 (Millipore), and then the solvent was replaced with PBS(−) containing 0.05% NaN₃ using PD-10 Desalting columns (Amersham Biosciences. The absorbance at 280 nm was measured with ND-1000 Spectrophotometer (NanoDrop), and the concentrations were determined by the method of Pace et al. (Protein Science (1995) 4: 2411-2423).

### Assessment of the Activity of Chimeric NS22

The activity of neutralizing hIL-31 was assessed using the hNR10/hOSMR/BaF3 cell line, which grows in an hIL-31 dose-dependent manner, as described below.

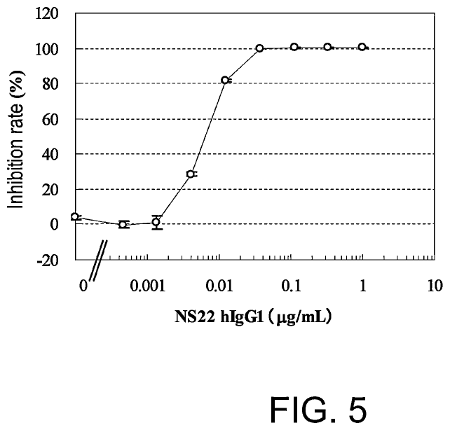

hNR10/hOSMR/BaF3 cells were prepared at 1.5×10⁵ cells/ml using RPMI1640 medium (GIBCO) containing 10% FBS (MOREGATE) and 1% Penicillin-Streptomycin (Invitrogen). hIL-31 (R&D Systems) was added to an aliquot of the cells to a final concentration of 4 ng/ml (IL-31(+); final conc.: 2 ng/ml). The remaining cell suspension was used as IL-31(−). The purified NS22 was adjusted to 2 g/ml using the medium, and eight serial dilutions were prepared at a common dilution ratio of 3 (final conc.: 1 g/ml or less). 50 1 each of the cell suspension and the dilution of chimeric NS22 (human 1,) was added to each well of 96-well flat-bottom plates (CORNING), and the cells were cultured in a 5% CO₂ incubator at 37° C. for two days. After culture, 20 1 of a mixture of equal amounts of Cell Counting Kit-8 (Dojindo) and PBS was added to each well, and the absorbance (450 nm/620 nm) was measured (TECAN, SUNRISE CLASSIC). After the reaction was allowed to continue for two hours in a 5% CO₂ incubator at 37° C., the absorbance was measured again. The neutralizing activity of NS22 was presented as an inhibition rate using a value obtained by subtracting the 0-hour value from the 2-hour value. The result showed that NS22 suppressed the IL-31-induced growth of the hNR 10/hOSMR/BaF3 cell line in a concentration-dependent manner. This demonstrates that NS22 has a neutralizing activity against the human IL-31 signaling (FIG. 5).

The IL-31-neutralizing activity was assessed as described below using the DU145 cell line (human prostate cancer cell line), in which IL-6 production is induced upon IL-31 stimulation.

DU145 cells were prepared at 2.5×10⁵ cells/ml in MEM (Invitrogen) containing 10% FBS (MOREGATE), 2 mmol/l L-glutamine (Invitrogen), and 1 mmol/l sodium pyruvate (SIGMA), and 200-□1 aliquots were dispensed into each well of 48-well plates (CORNING). The cells were incubated at 37° C. under 5% CO₂ overnight. The purified chimeric NS22 (human □1,□) was diluted to 100 □g/ml with MEM containing 10% FBS, 2 mmol/l L-glutamine, and sodium pyruvate. Using this solution, six serial dilutions were prepared at a common dilution ratio of 5. Each dilution was combined with 100 ng/ml human interleukin-31 (R&D systems) at a ratio of 1:1, and a 50-□1 aliquot was added to each well. After two days of culture at 37° C. under 5% CO₂, the concentration of IL-6 in the culture supernatant was determined using DuoSet ELISA Development kit (R&D systems). The neutralizing activity of NS22 was assessed by determining the inhibition rate (%). Specifically, assuming the IL-6 concentration in the absence of IL-31 (A) as the maximal inhibitory activity (100% inhibition) and the IL-6 concentration in the presence of IL-31 without NS22 (B) as no inhibitory activity (0% inhibition), the IL-6 concentration in the presence of IL-31 and NS22 (C) was determined according to the following formula:

$$Inhibition rate ( \% ) = ( B - C ) / ( B - A ) \times 100$$

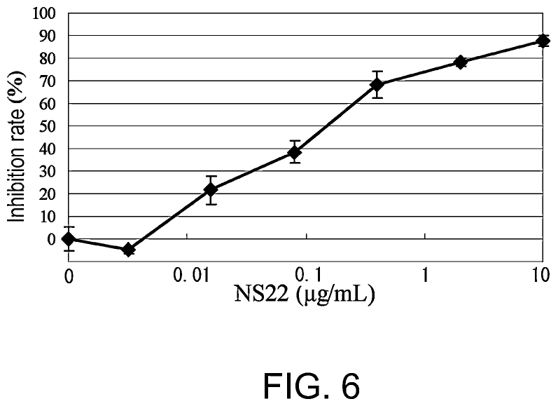

The result showed that NS22 suppressed the IL-31-induced IL-6 production in the DU145 cell line in a concentration-dependent manner and thus demonstrated that NS22 had a neutralizing activity against the human IL-31 signaling (FIG. 6).

Assessment of Competition of Chimeric Anti-NR 10 Antibody with IL-31

Human IL-31 (R&D Systems) was labeled with FMAT Blue Monofunctional Reactive Dye (Applied Biosystems). 100 □l of hIL-31 prepared at 0.5 mg/ml using 50 mM sodium phosphate buffer (pH 8.0) was mixed with 5.25 □l of 25 nmoles FMAT Blue dissolved in DMSO (Junsei). After vortexing, the mixture was allowed to stand at room temperature for 15 minutes. The FMAT Blue-conjugating reaction with hIL-31 was terminated by adding 5 □l of 1 M Tris-HCl (pH 7.4) and 1.1 □l of 10% Tween20, and then FMAT Blue-labeled hIL-31 and unreacted FMAT Blue were separated by gel filtration using Superdex 75 (GE Healthcare, 17-0771-01) column with 0.1% Tween20/PBS developing solution.

Antibodies were assessed for the activity of inhibiting the IL-31/NR10 binding by using hNR 10-expressing CHO cells as described below.

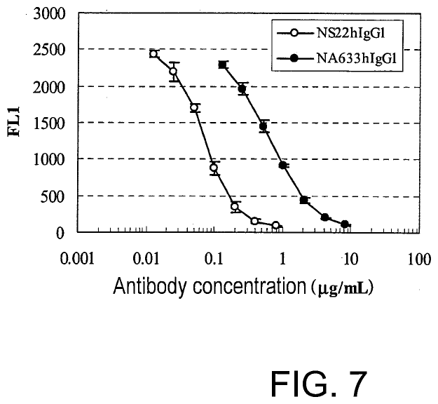

NS22 and NA633 (the constant region of each is □1,□) were diluted at an appropriate concentration using Assay buffer (10 mM HEPES, 140 mM NaCl, 2.5 mM CaCl₂, 3 mM MgCl₂, 2% FBS, 0.01% NaN₃), and then seven serial dilutions were prepared at a common dilution ratio of 2. The dilutions were added at 40 □l/well to plates (96-Well FMAT Plates; Applied Biosystems). Then, FMAT Blue-labeled hIL-31 was diluted 400 times with Assay buffer and added at 20 □l/well. Finally, cell suspensions adjusted to 2.5×10⁵ cells/ml using Assay buffer were added at 40 □l/well (final 1×10⁴ cells/well). Two hours after addition of cells, the fluorescence (FL1) was determined using the 8200 Cellular Detection System (Applied Biosystems). The result showed that NS22 inhibited the binding of hIL-31/hNR10 in a dose-dependent manner, and demonstrated that its activity was superior to that of NA633 (FIG. 7).

### Example 3

### Competition of Anti-NR10 Antibody Against NR10

The antibody NS22 purified from a hybridoma culture supernatant was labeled with FMAT Blue (Applied Biosystems, 4328853). 170 □l of NS22 prepared at 1 mg/ml in PBS was mixed with 17 1 of 1 M NaHCO₃ solution and 3.4 □l of FMAT Blue (17 nmoles) dissolved in DMSO. After vortexing, the mixture was allowed to stand at room temperature for 30 minutes. The FMAT Blue conjugating reaction with NS22 was terminated by adding 8 □l of 1 M Tris-HCl (pH 7.4) and 1.9 □l of 1% Tween 20, and then FMAT Blue-labeled NS22 (FMAT Blue-NS22) and unreacted FMAT Blue were separated by gel filtration using Superdex 75 (GE Healthcare, 17-0771-01) column with 0.01% Tween20/PBS developing solution.

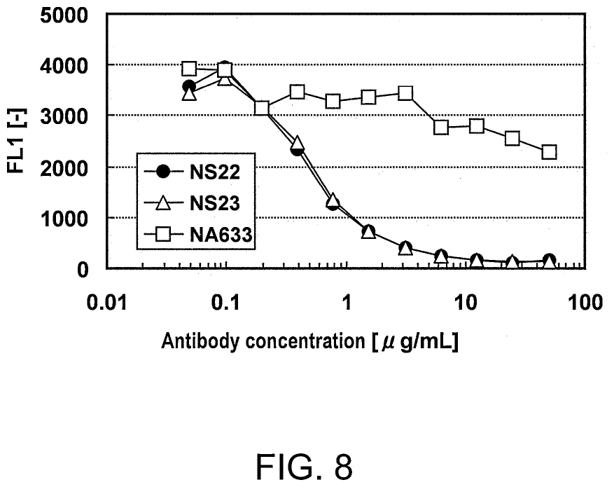

Each antibody was examined for inhibition of the binding of the prepared FMAT Blue-NS22 to hNR10-expressing CHO cells (Referential Example 3) using the 8200 Cellular Detection System (Applied Biosystems, 4342920). The chimeric anti-NR10 antibodies (the constant region of each is 1,) were added at various concentrations to each well containing 7500 cells and 8.8×10⁻² g/ml FMAT Blue-NS22. The cells were allowed to stand in the dark for four hours, and then the fluorescent signal from FMAT Blue bound to the cells was measured. The reaction was carried out in 10 mM Hepes-KOH containing 2.5 mM CaCl₂, 3 mM MgCl₂, 140 mM NaCl, 2% FBS, and 0.01% NaNO₃. The result is shown in FIG. 8. The fluorescence value FL1, which represents the binding of FMAT Blue-NS22 to NR10-expressing cells, was reduced with the increase in the concentration of antibody NS22 or NS23. On the other hand, FLI was hardly reduced with the increase in the concentration of antibody NA633 (Referential Example 6) (FIG. 8).

### Example 4

### Humanization of NS22 Antibody

### Selection of Each Framework Sequence

The variable regions of mouse NS22 antibody were compared with human germline sequences. FR sequences used for humanization are summarized in Table 2. CDRs and FRs were determined based on the Kabat numbering. The humanized variable region sequences of H chain composed of FR1, FR2, FR3_1, and FR4, and composed of FR1, FR2, FR3_2, and FR4, which are listed in Table 2, are designated as H0-VH (SEQ ID NO: 50) and H1-VH (SEQ ID NO: 112), respectively. Meanwhile, the sequence of L chain composed of FR1, FR2, FR3, and FR4 is designated as L0 (SEQ ID NO: 52).

### Preparation of Variable Region for Humanized NS22 H0L0

Synthetic oligo DNAs were designed for each of the H and L chains to construct the variable regions of humanized NS22 in which the CDRs of NS22 are grafted onto the FRs used for humanization. The respective synthetic oligo DNAs were mixed, and then subjected to assembly PCR to construct a gene encoding the variable region of humanized NS22. The assembly PCR was carried out using KOD-Plus (TOYOBO) according to the following conditions. A reaction mixture containing 10 pmol synthetic oligo DNAs and the appended PCR Buffer, dNTPs, MgSO₄, and KOD-Plus was heated at 94° C. for five minutes. The mixture was then subjected to two PCR cycles of 94° C. for two minutes, 55° C. for two minutes, and 68° C. for two minutes. Then, 10 pmol each of a primer in which a restriction site and Kozak sequence has been added to the 5′ end of the variable region, and a primer in which a restriction site has been added to the 3′ end of the variable region, was added and subjected to 35 PCR cycles of 94° C. for 30 seconds, 55°° C. for 30 seconds, and 68° C. for one minute to yield a amplified fragment. The resulting amplified fragment was cloned into TOPO TA Cloning vector (TOYOBO), and its nucleotide sequence was determined by sequencing. The constructed variable regions were combined with the constant regions to prepare H0-SKSC (SEQ ID NO: 54) and L0 (SEQ ID NO: 56). The resulting construct was inserted into an expression vector capable of expressing the inserted gene in animal cells. The nucleotide sequence of each DNA fragment was determined using BigDye Terminator Cycle Sequencing Kit (Applied Biosystems) with ABI PRISM 3730xL DNA Sequencer or ABI PRISM 3700 DNA Sequencer (Applied Biosystems) according to the method described in the appended instruction manual.

### Preparation of Variable Region for Humanized NS22 H1

H1-SKSC (SEQ ID NO: 130) was generated by substituting the glutamine (E) at Kabat-numbering position 73 in FR3 of H0-SKSC (SEQ ID NO: 54) with lysine (K). The mutant was prepared using commercially available QuikChange Site-Directed Mutagenesis Kit (Stratagene) according to the appended instruction manual.

### Expression of IgG-Converted Antibody

Antibody expression was performed by the method described below. Human fetal renal cancer cell-derived cell line HEK293H (Invitrogen) was suspended in DMEM (Invitrogen) containing 10% fetal bovine serum (Invitrogen), and 10 ml of cells at a density of 5-6 x 105 cells/ml was seeded onto dishes for adherent cells (10 cm in diameter; CORNING). The cells were incubated in a CO₂ incubator (37° C., 5% CO₂) for one whole day and night. Then, the medium was removed by aspiration, and 6.9 ml of CHO-S-SFMII medium (Invitrogen) was added to the cells. The prepared plasmid DNA mixture (13.8 □g in total) was mixed with 20.7 □1 of 1 □g/ml polyethyleneimine (Polysciences Inc.) and 690 □1 of CHO-S-SFMII medium, and allowed to stand at room temperature for 10 minutes. The mixture was added to the cells in each dish, and the cells were incubated in a CO₂ incubator (5% CO₂, 37° C.) for four to five hours. Then, 6.9 ml of CHO-S-SFMII medium (Invitrogen) was added, and the cells were incubated in a CO₂ incubator for three days. The culture supernatant was collected and centrifuged (approx. 2000 g, five minutes, room temperature) to remove the cells. The supernatant was then sterilized by filtration through 0.22-□m filter MILLEX®-GV (Millipore). Each sample was stored at 4° C. until use.

Purification of IgG-Converted ntibody

50 □1 of rProtein A Sepharose™ Fast Flow (Amersham Biosciences) suspended in TBS was added to the obtained culture supernatant, and mixed by inversion at 4° C. for four hours or more. The solution was transferred to 0.22-□m filter cup of Ultrafree®-MC (Millipore). After three washes with 500 □l of TBS, rProtein A Sepharose™ resin was suspended in 100 □l of aqueous solution of 50 mM sodium acetate (pH 3.3), and allowed to stand for three minutes to elute the antibody. The solution was immediately neutralized by adding 6.7 □l of 1.5 M Tris-HCl (pH 7.8). The elution was performed twice and 200 □l of purified antibody was obtained. 2 □l of the antibody-containing solution was subjected to ND-1000 Spectrophotometer (NanoDrop)(Thermo Scientific NanoDrop™ 1000 Spectrophotometer (Thermo Scientific)) or 50 □l was subjected to Spectrophotometer DU-600 (BECKMAN) to measure absorbance at 280 nm, and the antibody concentration was calculated by the method of Pace et al. (Protein Science (1995) 4: 2411-2423).

Measurement of Competition with IL-31 Using FMAT

Antibodies were assessed for the activity of inhibiting the IL-31/NR10 binding by using hNR 10-expressing CHO cells as described below. The chimeric NS22 antibody and NS22_H0L0 (H chain, H0-SKSC/SEQ ID NO: 54; L chain, L0/SEQ ID NO: 56) were diluted at an appropriate concentration using Assay buffer (10 mM HEPES, 140 mM NaCl, 2.5 mM CaCl₂, 3 mM MgCl2, 2% FBS, 0.01% NaN₃, pH7.4), and further eight serial dilutions were prepared at a common dilution ration of 2. The dilutions were added at 40 □l/well to plates (96-Well FMAT Plates, Applied Biosystems). Then, FMAT Blue-labeled hIL-31 was diluted 400 times with Assay buffer, and added at 20 □l/well. Finally, a cell suspension adjusted to 2.5×10⁵ cells/ml using Assay buffer was added at 40 □l/well (final 1×10⁴ cells/well). Two hours after addition of cells, the fluorescence (FL1) was measured using the 8200 Cellular Detection System (Applied Biosystems).

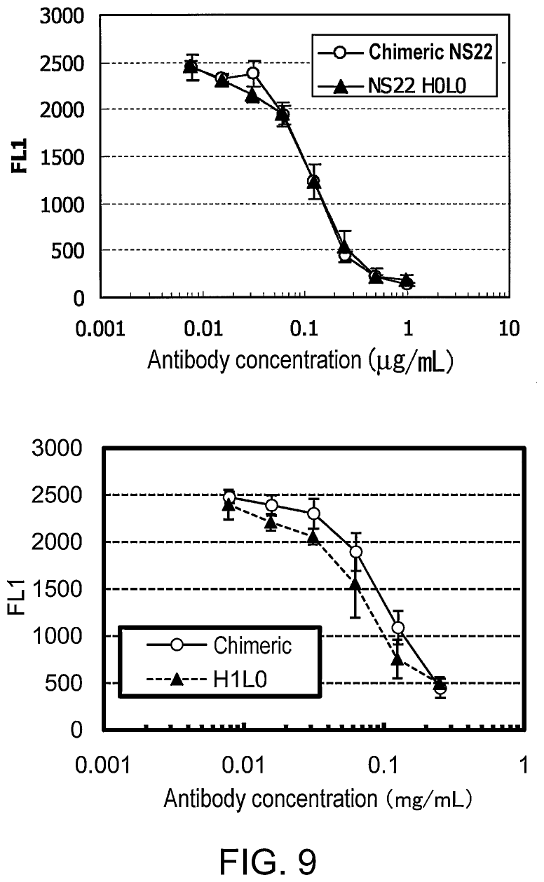

The result showed that, as shown in FIG. 9, humanized NS22 antibodies H0L0 (H chain, H0-SKSC/SEQ ID NO: 54; L chain, L0/SEQ ID NO: 56), and H1L0 (H chain, H1-SKSC/SEQ ID NO: 130; L chain, L0/SEQ ID NO: 56) exhibited a competition activity comparable to that of the chimeric antibody, suggesting that both H0L0 and H1L0 are humanized anti-IL-31 receptor antibodies. In addition, it is considered that the FRs used for H0L0 and H1L0 can be used for humanization.

Accordingly, all of the mutations in CDRs described in the Examples hereinafter can be introduced into both H0 and H1.

TABLE 2

| H0 | Germline | Human FR sequence |
|---|---|---|
| FR1 | Germline:hVH_1_46 (Accession No. X92343) | QVQLVQSGAEVKKPGASVKVSCKASGYTFT |
| | | (SEQ ID NO: 96) |
| FR2 | Germline:hVH_1_46 (Accession No. X92343) | WVRQAPGQGLEWMG (SEQ ID NO: 97) |
| FR3_1 | Germline:hVH_1_69 (Accession No. L22582) | RVTITADESTSTAYMELSSLRSEDTAVYYCAR |
| | | (SEQ ID NO: 98) |
| FR3_2 | Germline:hVH_1_69 (Accession No. Z27506) | RVTITADKSTSTAYMELSSLRSEDTAVYYCAR |
| | | (SEQ ID NO: 131) |
| FR4 | Germline:JH1 | WGQGTLVTVSS (SEQ ID NO: 99) |
| L0 | Germline | Human FR sequence |
| FR1 | Germline:hVK_1_39 (Accession No. X59315) | DIQMTQSPSSLSASVGDRVTITC |
| | | (SEQ ID NO: 100) |
| FR2 | Germline:hVK_1_39 (Accession No. X59315) | WYQQKPGKAPKLLIY (SEQ ID NO: 101) |
| FR3 | Germline:hVK_1_39 (Accession No. X59315) | GVPSRFSGSGSGTDFTLTISSLQPEDFATYYC |
| | | (SEQ ID NO: 102) |
| FR4 | Germline:JK4 | FGGGTKVEIK (SEQ ID NO: 103) |

### Example 5

### Heterogeneity-Reducing Effect of Novel Constant Regions M14 and M58 in Humanized Anti-IL31 Receptor Antibody

As shown in Referential Examples 7 to 9, it was demonstrated that the conversion of the constant region from IgG2 to M14 or M58 in the huPM1 antibody, a humanized anti-IL-6 receptor antibody, could reduce the heterogeneity derived from the IgG2 hinge region without loss of stability. Thus, humanized anti-IL-31 receptor antibodies were also tested to assess whether the heterogeneity can be reduced by converting their constant regions from the wild-type IgG2 to M14 or M58.

H0-M14, H0-M58, H0-IgG1, and H0-IgG2, which were generated by combining IgG1 (SEQ ID NO: 60), IgG2 (SEQ ID NO: 132), M14 (SEQ ID NO: 129) and M58 (SEQ ID NO: 128) generated in Referential Examples 8 and 9, with H chain variable region H0 (H0-VH/SEQ ID NO: 50) of humanized anti-IL-31 receptor antibody generated in Example 4, were used as H chains, and L0 (L0/SEQ ID NO: 56) produced in Example 4 was used as an L chain, to generate H0L0-IgG1 (H chain, H0-IgG1/SEQ ID NO: 133; L chain, L0/SEQ ID NO: 56), H0L0-IgG2 (H chain, H0-IgG2/SEQ ID NO: 134; L chain, L0/SEQ ID NO: 56), H0L0-M14 (H chain, H0-M14/SEQ ID NO: 135; L chain, L0/SEQ ID NO: 56), and H0L0-M58 (H chain, H0-M58/SEQ ID NO: 136; L chain, L0/SEQ ID NO: 56). Each antibody was expressed and purified by the method described in Example 4.

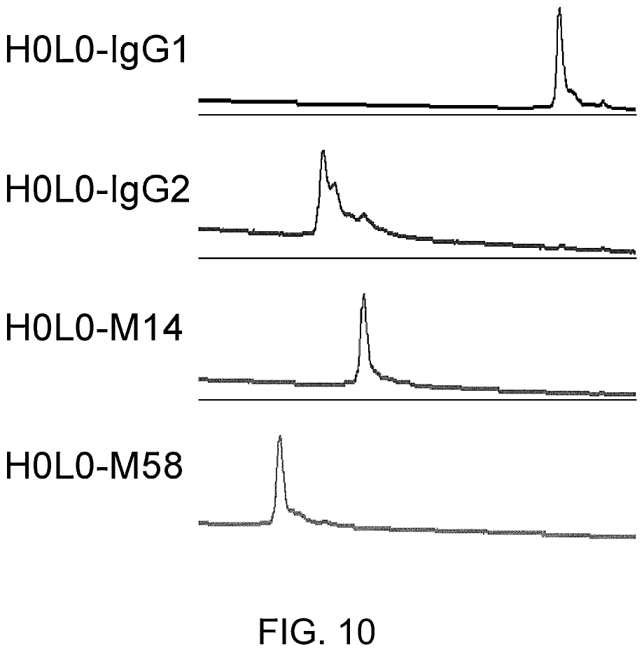

The heterogeneity was assessed by cation exchange chromatography. The prepared antibodies were assessed for heterogeneity using ProPac WCX-10 (Dionex) column, 20 mM sodium acetate (pH 5.0) as mobile phase A, and 20 mM sodium acetate/1M NaCl (pH 5.0) as mobile phase B, with an appropriate flow rate and gradient. The result of assessment by cation exchange chromatography (IEC) is shown in FIG. 10.

As shown in FIG. 10, the heterogeneity was increased by conversion of the constant region from IgG1 to IgG2 in the anti-IL-31 receptor antibody, and the heterogeneity can be reduced by conversion of the constant region to M14 or M58 in any antibody.

### Example 6

### Pharmacokinetics Improving Effect of Novel Constant Region M58 in anti-IL-31 Receptor Antibodies

As shown in Referential Example 9, conversion of the constant region from IgG1 to M58 in anti-IL-6 receptor antibody huPM1 was found to improve its human FcRn-binding activity and the pharmacokinetics in human FcRn transgenic mice. Thus, anti-IL-31 receptor antibodies were also tested to assess whether conversion of the constant region to M58 improves their pharmacokinetics.

H0L0-IgG1 (H chain: H0-IgG1/SEQ ID NO: 133; L chain: L0/SEQ ID NO: 56) and H0L0-M58 (H chain: H0-M58/SEQ ID NO: 136; L chain L0/SEQ ID NO: 56) prepared as described in Examples 4 and 5 were assessed for the human FcRn-binding activity by the method described in Referential Example 9. The result is shown in Table 3.

TABLE 3

| | KD(μM) |
|---|---|
| H0L0-IgG1 | 1.07 |
| H0L0-M58 | 0.91 |

As shown in Table 3, conversion of the constant region from IgG1 to M58 also improved the human FcRn-binding activity of the anti-IL-31 receptor antibody H0L0 as in the anti-IL-6 receptor antibody hPM1. This suggests that conversion of the constant region from IgG1 to M58 may improve the pharmacokinetics of anti-IL-31 receptor antibody in human.

### Example 7

### Identification of Mutation Sites Reducing the Isoelectric Point Production of Mutants

Each mutant was produced by the method described in Example 4 or by assembly PCR. In the method using assembly PCR, oligo DNAs are synthesized based on forward and reverse sequences including an altered site. Forward oligo DNA including an altered site and reverse oligo DNA binding to the vector in which the gene to be altered was inserted were combined, and reverse oligo DNA including an altered site and forward oligo DNA binding to the vector in which the gene to be altered was inserted were combined. PCR was carried out using PrimeSTAR (Takara) to produce 5′-end and 3′-end fragments including the altered site. The two fragments were assembled by assembly PCR to produce each mutant. The produced mutant was inserted into an expression vector capable of expressing the insert gene in animal cells. The nucleotide sequence of the resulting expression vector was determined by a method known to those skilled in the art. Antibodies were produced and purified by the method described in Example 4.

### Identification of Mutation Sites

To improve the pharmacokinetics of H0L0 (H chain, H0-SKSC/SEQ ID NO: 54; L chain, L0/SEQ ID NO: 56), altered sites capable of reducing the isoelectric point of the variable region were examined. Screening of mutation sites in the variable regions predicted from the three-dimensional structure model revealed mutation sites that would decrease the isoelectric point of the variable regions without significantly reducing its binding to NR10. These are summarized in Table 4 (Hp5-VH/SEQ ID NO: 137, Hp7-VH/SEQ ID NO: 138, Hp8-VH/SEQ ID NO: 139, Hp6-VH/SEQ ID NO: 140, Hp9-VH/SEQ ID NO: 141, Hp1-VH/SEQ ID NO: 142, Hp13-VH/SEQ ID NO: 143, Lp1-VL/SEQ ID NO: 144, Lp2-VL/SEQ ID NO: 145, Lp3-VL/SEQ ID NO: 146, Lp4-VL/SEQ ID NO: 147, Lp7-VL/SEQ ID NO: 148, Lp5-VL/SEQ ID NO: 149, Lp6-VL/SEQ ID NO: 150). Each variant was produced and purified by the method described in Example 4.

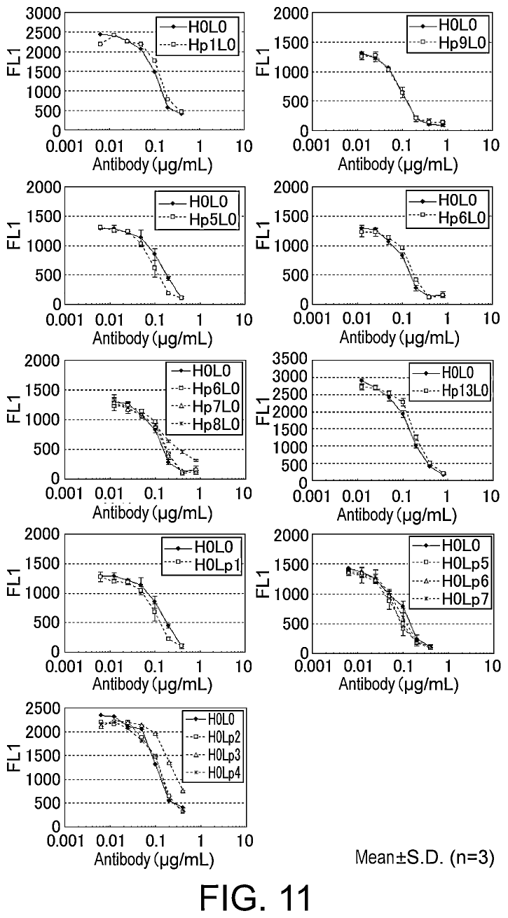

Each variant was tested for the activity of inhibiting the hIL-31/hNR10 binding by using FMAT. The test was carried out according to the method as described in Example 4. As shown in FIG. 11, the competition activity of each variant was not greatly reduced as compared to that of H0L0.

TABLE 4

| | | | Mutation site | H0 | Amino acid | Sequence after |
|---|---|---|---|---|---|---|
| Name | Type | H0 sequence | (kabat No) | sequence | after mutation | mutation |
| Hp5 | FR2 | WVRQAPGQGLEWMG | 38 | R | Q | WVQQSPGQGLEWMG |
| | | (SEQ ID NO: 97) | 40 | *A | S | (SEQ ID NO: 120) |
| Hp7 | CDR2 | LINPYNGGTSYNQKFKG | 50 | L | E | EINPYNGGTSYNQKFKG |
| | | (SEQ ID NO: 10) | | | | (SEQ ID NO: 113) |
| Hp8 | CDR2 | LINPYNGGTSYNQKFKG | 52 | N | D | LIDPYNGGTSYNQKFKG |
| | | (SEQ ID NO: 10) | | | | (SEQ ID NO: 114) |
| Hp6 | CDR2 | LINPYNGGTSYNQKFKG | 61 | Q | D | LINPYNGGTSYNQKFKG |
| | | (SEQ ID NO: 10) | | | | (SEQ ID NO: 115) |
| Hp9 | CDR2 | LINPYNGGTSYNQKFKG | 62 | K | Q | LINPYNGGTSYNQQFKG |
| | | (SEQ ID NO: 10) | | | | (SEQ ID NO: 116) |
| Hp1 | CDR2 | LINPYNGGTSYNQKFKG | 64 | K | Q | LINPYNGGTSYNQKFQG |
| | | (SEQ ID NO: 10) | | | | (SEQ ID NO: 117) |
| Hp13 | CDR2 | LINPYNGGTSYNQKFKG | 64 | K | Q | LINPYNGGTSYNQKFQD |
| | | (SEQ ID NO: 10) | 65 | G | D | (SEQ ID NO: 119) |
| | | | Mutation site | L0 | Amino acid | Sequence after |
| Name | Type | L0 sequence | (kabat No) | sequence | after mutation | mutation |
| Lp1 | CDR1 | RTSENIYSFLA | 24 | R | Q | QTSENIYSFLA |
| | | (SEQ ID NO: 13) | | | | (SEQ ID NO: 121) |
| Lp2 | CDR1 | RTSENIYSFLA | 28 | N | D | RTSEDIYSFLA |
| | | (SEQ ID NO: 13) | | | | (SEQ ID NO: 122) |
| Lp3 | CDR2 | NAKTLAK | 50 | N | D | DAKTLAK |
| | | (SEQ ID NO: 14) | | | | (SEQ ID NO: 123) |
| Lp4 | CDR2 | NAKTLAK | 52 | K | Q | NAQTLAK |
| | | (SEQ ID NO: 14) | | | | (SEQ ID NO: 124) |
| Lp7 | CDR2 | NAKTLAK | 54 | L | E | NAKTEAK |
| | | (SEQ ID NO: 14) | | | | (SEQ ID NO: 125) |
| Lp5 | CDR2 | NAKTLAK | 56 | K | Q | NAKTLAQ |
| | | (SEQ ID NO: 14) | | | | (SEQ ID NO: 126) |
| Lp6 | CDR2 | NAKTLAK | 56 | K | D | NAKTLAD |
| | | (SEQ ID NO: 14) | | | | (SEQ ID NO: 127) |

Asterisk (*) in Table 4 above indicates a site that was not relevant to the isoelectric point but altered for conversion into a human sequence.

Examples of the humanized NS22 antibodies whose isoelectric point has been reduced by combining these alterations include Hp3Lp15 (H chain: Hp3-SKSC/SEQ ID NO: 151; L chain: Lp15/SEQ ID NO: 152). Affinity for NR10, isoelectric point, and plasma retention in mice were compared between Hp3Lp15 and H0L0.

### Measurement of Affinity

The affinity of each antibody for NR10 was determined by the method described in Referential Example 10.

The result of affinity measurement is shown in Table 5. The affinity of Hp3Lp15 was shown to be almost the same as that of H0L0.

TABLE 5

| | ka (1/Ms) | kd (1/s) | KD (M) |
|---|---|---|---|
| H0L0 | 3.7E+05 | 1.2E−03 | 3.3E−09 |
| Hp3Lp15 | 4.2E+05 | 1.6E−03 | 3.9E−09 |

### Measurement of Isoelectric Point

Each antibody was analyzed by isoelectric focusing to assess changes in the isoelectric point of the whole antibody due to the amino acid alterations in its variable region. Isoelectric focusing was performed by the following method.

Phast-Gel Dry IEF gel (Amersham Biosciences) was swollen in Phastsystem Cassette (Amersham Biosciences) for about 30 minutes using the swelling solution shown below.

| | |
|---|---|
| MilliQ water | 1.5 ml |
| Pharmalyte 5-8 for IEF (Amersham Biosciences) | 100 □l |

Electrophoresis was carried out in PhastSystem (Amersham Biosciences) using the swollen gel according to the program indicated below. The samples were loaded onto the gel in Step 2. Calibration Kit for pI (Amersham Biosciences) was used as a pI marker.

| | | | | | |
|---|---|---|---|---|---|
| Step 1: | 2000 V | 2.5 mA | 3.5 W | 15° C. | 75 Vh |
| Step 2: | 200 V | 2.5 mA | 3.5 W | 15° C. | 15 Vh |
| Step 3: | 2000 V | 2.5 mA | 3.5 W | 15° C. | 410 Vh |

After electrophoresis, the gel was fixed with 20% TCA, and then silver-stained using the Silver Staining Kit, Protein (Amersham Biosciences), according to the protocol attached to the kit. After staining, the isoelectric point of the sample (the whole antibody) was calculated from the known isoelectric points of the pI markers.

The result of isoelectric point measurement by isoelectric focusing showed that the isoelectric point of H0L0 was about 7.8, and the isoelectric point of Hp3Lp15 was about 5.5, showing that the isoelectric point of Hp3Lp15 was decreased by about 2.3 as compared to H0L0.When the theoretical isoelectric point of the variable region VH/VL was calculated by GENETYX (GENETYX CORPORATION), the theoretical isoelectric points of the variable regions of H0L0 and Hp3Lp15 were 7.76 and 4.63, respectively. Thus, the theoretical isoelectric point of Hp3Lp15 was decreased by 3.13 as compared to H0L0.

Assessment of Pharmacokinetics of Antibody with Reduced Isoelectric Point Using Mice

In order to assess the plasma retention of Hp3Lp15, a modified antibody with reduced isoelectric point, the plasma retention of H0L0 and Hp3Lp15 was compared in normal mice. A single dose of H0L0 or Hp3Lp15 was intravenously administered at 1 mg/kg to mice (C57BL/6J, Charles River Japan, Inc.) to compare the time course of the plasma concentration. The plasma concentrations were determined by ELISA. Appropriate concentrations of a calibration sample and test plasma samples were dispensed into immunoplates (Nunc-Immuno Plate, MaxiSorp (Nalge Nunc International)) coated with anti-human IgG (Fc-specific) antibody (Sigma). The samples were allowed to stand at room temperature for one hour. After reaction with Goat Anti-Human IgG-ALP (Sigma) at room temperature for one hour, color developing reaction was carried out using BluePhos Microwell Phosphatase Substrates System (Kirkegaard & Perry Laboratories) as a substrate. The absorbance at 650 nm was measured with a microplate reader. The plasma concentrations were determined based on the absorbance of the calibration curve using the analytical software SOFTmax PRO (Molecular Devices).

Pharmacokinetic parameters (AUC and systemic clearance (CL)) were calculated from the obtained time-course data of the plasma concentration using the pharmacokinetics analysis software WinNonlin (Pharsight). The parameters are shown in Table 6. AUC and the clearance of Hp3Lp15 after the intravenous administration were reduced by about 14% and about 12%, respectively, as compared to H0L0. Thus, it was demonstrated that Hp3Lp15, in which the isoelectric point of H0L0 has been reduced, had improved pharmacokinetics.

TABLE 6

| | |
|---|---|
| AUC(μg · d/kg) | CL(ml/d/kg) |

| | Mean | SD | Mean | SD |
|---|---|---|---|---|
| H0L0 | 281.8 | 13.1 | 3.6 | 0.2 |
| Hp3Lp15 | 321.1 | 26.1 | 3.1 | 0.3 |

### Example 8

### Effect of Combinations of Variable Region and Constant Region on the Biological Activity

In order to assess the effects of different constant regions on the biological activity, the following variants were produced.

SKSC (SEQ ID NO: 62) and M58 (SEQ ID NO: 128), constant regions prepared in Referential Examples 7 and 9, were combined with Hp3 (Hp3-VH/SEQ ID NO: 167), a variable region prepared in Example 7, to produce Hp3-M58 (SEQ ID NO: 240) and Hp3-SKSC (SEQ ID NO: 151) as H chains. The prepared H chains were combined with Lp15 (Lp15/SEQ ID NO: 152), an L chain prepared in Example 7, to produce Hp3Lp15-SKSC (H chain, Hp3-SKSC/SEQ ID NO: 151; L chain, Lp15/SEQ ID NO: 152) and Hp3Lp15-M58 (H chain, Hp3-M58/SEQ ID NO: 240; L chain, Lp15/SEQ ID NO: 152). Each antibody was expressed and purified by the method described in Example 4.

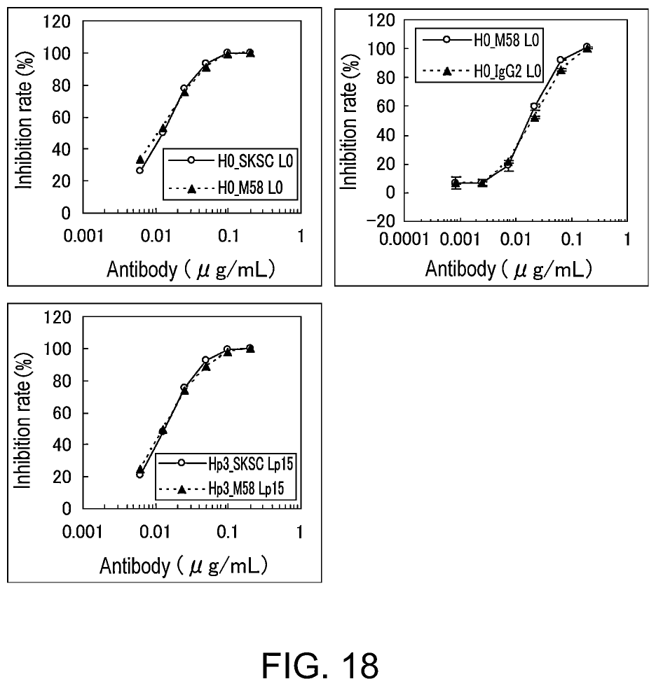

The antibodies produced as described above, H0L0-SKSC (H chain, H0-SKSC/SEQ ID NO: 54; L chain, L0/SEQ ID NO: 56) prepared using the constant region SKSC (SEQ ID NO: 62) described in Referential Example 7, and H0L0-M58 (H chain, H0-M58/SEQ ID NO: 136; L chain, L0/SEQ ID NO: 56) and H0L0-IgG2 (H chain, H0-IgG2/SEQ ID NO: 134; L chain, L0/SEQ ID NO: 56) prepared in Example 5, were assessed for the biological activity by the method described in Example 2 using BaF/NR10. The result is summarized in FIG. 18.

As shown in FIG. 18, no significant difference in the biological activity was detected between the constant regions. Since the biological activity was not affected when combining the two variable regions H0 and Hp3 with each constant region, combining variable regions created in future with any constant region would not result in alteration in the biological activity.

### Example 9

### Identification of Mutation Sites Suppressing Degradation by Thermal Acceleration Study

Antibodies used for pharmaceuticals have heterogeneity even though they are monoclonal antibodies obtained from clones derived from single antibody-producing cells. Such antibody heterogeneity is known to result from modification such as oxidation or deamidation, and to be increased during long-term storage or upon exposure to stress conditions, such as heat stress or light stress (see “Heterogeneity of Monoclonal Antibodies”, Journal of pharmaceutical sciences, vol. 97, No. 7, 2426-2447). However, when an antibody is developed as a pharmaceutical, physical properties of the protein, particularly homogencity and stability, are highly important. Thus, it is desired that the heterogeneity of desired/related substances be reduced and the substance be composed of a single substance as much as possible. In this context, the experiment described below was conducted to assess the antibody heterogencity under stress conditions and to reduce the heterogeneity.

To assess degradation products, an accelerated sample of H0L0 (H chain, H0-SKSC/SEQ ID NO: 54; L chain, L0/SEQ ID NO: 56) was prepared by the method described below. The prepared accelerated sample and non-accelerated sample (initial) were analyzed by cation exchange chromatography using the method described below.

### Method for Preparing Accelerated Samples

### Buffer: PBS

Antibody concentration: 0.2 to 1.0 mg/ml
Acceleration temperature: 60°° C.
Acceleration period: one day

### Method for Analysis by Cation Exchange Chromatography

Column: ProPac WCX-10, 4×250 mm (Dionex)
Mobile phase: (A) 25 mmol/l MES/NaOH, pH 6.1
   (B) 25 mmol/l MES/NaOH, 250 mmol/l NaCl, pH 6.1
Flow rate: 0.5 ml/min
Column temperature: 40°° C.
Gradient: % B 0 to 0 (0-5 min)→0 to 30 (5-80 min)
Detection: 280 nm

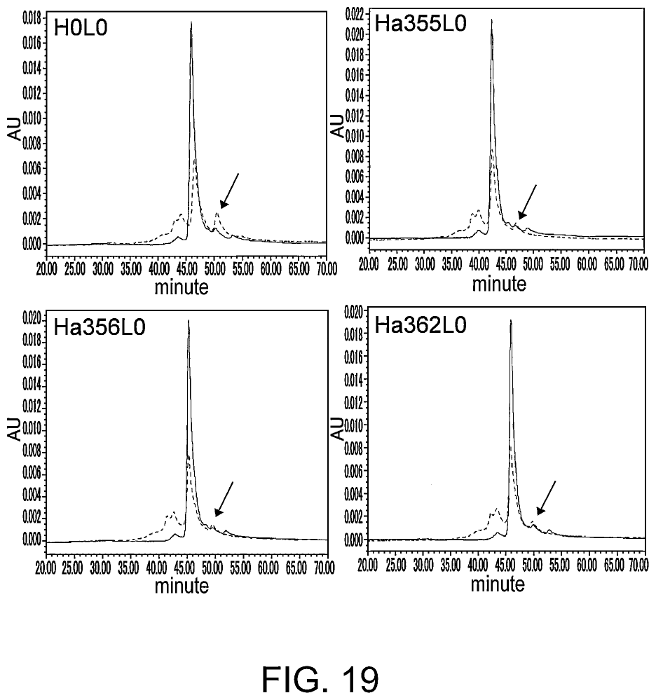

The resulting chromatograms for H0L0 samples before and after acceleration are shown in FIG. 19. The H0L0 sample after acceleration had a tendency to show an increased basic peak.

Then, screening was carried out to reduce this peak. As a result, Ha355, Ha356, Ha360, and Ha362 were found. These H chain variants were combined with L0 to produce Ha355L0 (H chain, Ha355-SKSC/SEQ ID NO: 242; L chain, L0/SEQ ID NO: 56), Ha356L0 (H chain, Ha356-SKSC/SEQ ID NO: 243; L chain, L0/SEQ ID NO: 56), Ha360L0 (H chain, Ha360-SKSC/SEQ ID NO: 244; L chain, L0/SEQ ID NO: 56), and Ha362L0 (H chain, Ha362-SKSC/SEQ ID NO: 245; L chain, L0/SEQ ID NO: 56). The sequence of each variant is shown in Table 7.

TABLE 7

| | | | Mutation site | H0 | Amino acid | Sequence after |
|---|---|---|---|---|---|---|
| Name | Type | H0 sequence | (kabat No) | sequence | after mutation | mutation |
| Ha355 | CDR3 | DGYDDGPYTMDY | 100d | M | L | DGYDDGPYTLET |
| | | (SEQ ID NO: 265) | 101 | D | E | (SEQ ID NO: 266) |
| | | | 102 | Y | T | |
| Ha356 | CDR3 | DGYDDGPYTMDY | 101 | D | E | DGYDDGPYTMET |
| | | (SEQ ID NO: 265) | 102 | Y | T | (SEQ ID NO: 267) |
| Ha360 | CDR3 | DGYDDGPYTMDY | 97 | Y | L | DGLDDGPYTMET |
| | | (SEQ ID NO: 265) | 101 | D | E | (SEQ ID NO: 268) |
| | | | 102 | Y | T | |
| Ha362 | CDR3 | DGYDDGPYTMDY | 97 | Y | L | DGLDDGPYTMES |
| | | (SEQ ID NO: 265) | 101 | D | E | (SEQ ID NO:269) |
| | | | 102 | Y | s | |

Each of the identified antibodies was expressed and purified by the method described in Example 4. As with H0L0, a accelerated sample of each prepared antibody was prepared, and analyzed by cation exchange chromatography. The result is shown in FIG. 19.

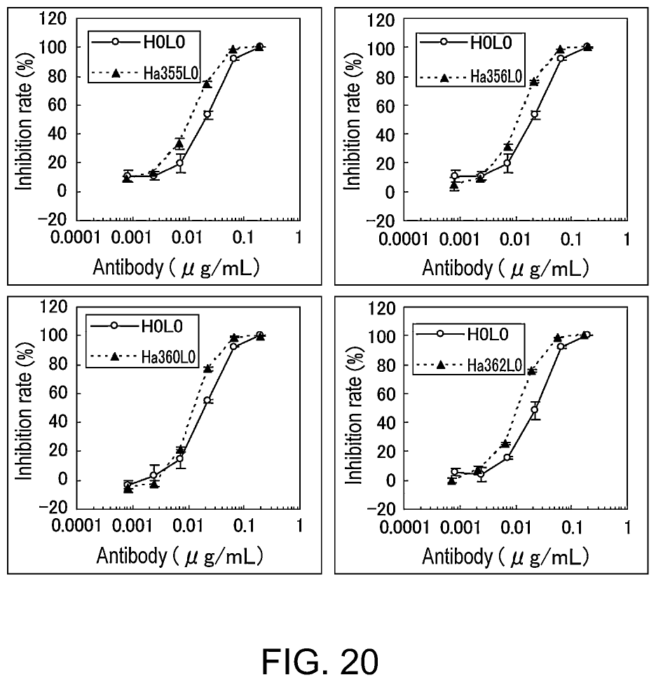

The result showed that the generation of the basic peak increased after acceleration was reduced in the modified antibody containing a substitution of aspartic acid with glutamic acid at position 101 in the H chain, as compared to H0L0. The modified antibodies were assessed for the biological activity by the method described in Example 2 using BaF/NR10. The result is shown in FIG. 20. As shown in FIG. 20, the biological activities of the modified antibodies were comparable to or stronger than that of H0L0. These findings demonstrated that the modifications of Ha355, Ha356, Ha360, and Ha362 suppressed the generation of degradation products by acceleration, and therefore are effective in improving the stability of antibody.

### Example 10

### Identification of Mutation Sites Increasing the Affinity

A library in which mutations were introduced into CDR sequences was constructed and examined to improve the affinity of H0L0 for NR10. As a result of screening of the library in which mutations were introduced into CDRs, mutations that improve the affinity for NR10 were found. The mutations are shown in Table 8. Each of the H chain variants Ha101-SKSC (SEQ ID NO: 246), Ha103-SKSC (SEQ ID NO: 247), Ha111-SKSC (SEQ ID NO: 248), Ha204-SKSC (SEQ ID NO: 249), and Ha219-SKSC (SEQ ID NO: 250) was combined with L0 (L0/SEQ ID NO: 56); and each of the modified L chains La134 (SEQ ID NO: 251), La130 (SEQ ID NO: 252), La303 (SEQ ID NO: 253), and La328 (SEQ ID NO: 254) was combined with H0 (H0-SKSC/SEQ ID NO: 54), to construct an antibody. Each variant was produced and purified by the method described in Example 4.

The affinity of each antibody for NR10 was assessed using Biacore. The result is shown in Table 9. The assay was carried out using the method described in Referential Example 10. As shown in Table 9, the KD value for each variant was found to be improved as compared to that of H0L0 (H chain, H0-SKSC/SEQ ID NO: 54; L chain, L0/SEQ ID NO: 56).

TABLE 8

| | | | Mutation site | H0 | Amino acid | Sequence |
|---|---|---|---|---|---|---|
| Name | Type | H0 sequence | (kabat No) | sequence | after mutation | after mutation |
| Ha101 | CDR1 | GYIMN | 33 | I | V | GYVMN |
| | | (SEQ ID NO: 270) | | | | (SEQ ID NO: 272) |
| Ha103 | CDR1 | GYIMN | 34 | M | I | GYIIN |
| | | (SEQ ID NO: 270) | | | | (SEQ ID NO: 273) |
| Ha111 | CDR1 | GYIMN | 34 | M | L | GYILN |
| | | (SEQ ID NQ: 270) | | | | (SEQ ID NO: 274) |
| Ha204 | CDR2 | LINPYNGGTSYNQKFKG | 58 | S | D | LINPYNGGTDYNQKFKG |
| | | (SEQ ID NO: 271) | | | | (SEQ ID NO: 275) |
| Ha219 | CDR2 | LINPYNGGTSYNQKFKG | 61 | Q | P | LINPYNGGTSYNPKFKG |
| | | (SEQ ID NO: 271) | | | | (SEQ ID NO: 276) |
| | | | Mutation site | L0 | Amino acid | Sequence |
| Name | Type | L0 sequence | (kabat No) | sequence | after mutation | after mutation |
| La134 | CDR1 | RTSENIYSFLA | 31 | S | R | RTSENIYRFLA |
| | | (SEQ ID NO: 277) | | | | (SEQ ID NO: 279) |
| La130 | CDR1 | RTSENIYSFLA | 31 | S | R | RTSENIYRFVA |
| | | (SEQ ID NO: 277) | 33 | L | V | (SEQ ID NO: 280) |
| Ls303 | CDR3 | QHHYESPLT | 93 | E | D | QHHYDSPLT |
| | | (SEQ ID NO: 278) | | | | (SEQ ID NO: 281) |
| La328 | CDR3 | QHHYESPLT | 94 | S | D | QHHYEDPLT |
| | | (SEQ ID NO: 278) | | | | (SEQ ID NO: 282) |
| La326 | CDR3 | QHHYESPLT | 97 | T | F | QHHYESPLF |
| | | (SEQ ID NO: 278) | | | | (SEQ ID NO: 283) |

TABLE 9

| Name | ka(1/Ms) | kd(1/s) | KD(M) |
|---|---|---|---|
| H0L0 | 1.9E+05 | 6.2E−04 | 3.2E−09 |
| Ha101L0 | 2.0E+05 | 3.1E−04 | 1.5E−09 |
| Ha103L0 | 2.2E+05 | 5.3E−04 | 2.4E−09 |
| Ha111L0 | 2.6E+05 | 5.6E−04 | 2.1E−09 |
| Ha204L0 | 3.7E+05 | 4.8E−04 | 1.3E−09 |
| Ha219L0 | 3.2E+05 | 9.6E−04 | 3.0E−09 |
| H0L0 | 1.5E+05 | 7.4E−04 | 5.1E−09 |
| H0La134 | 2.5E+05 | 4.4E−04 | 1.8E−09 |
| H0La130 | 2.6E+05 | 4.0E−04 | 1.5E−09 |
| H0La303 | 2.2E+05 | 4.6E−04 | 2.1E−09 |
| H0La328 | 1.8E+05 | 5.2E−04 | 2.9E−09 |
| H0La326 | 1.4E+05 | 5.2E−04 | 3.7E−09 |

Examples of combinations of these affinity-improving mutations with the isoelectric point-lowering mutations generated in Example 7 include, for example, Ha401La402 (H chain, Ha401-SKSC/SEQ ID NO: 255; L chain, La402/SEQ ID NO: 256) and H17L11 (H chain, H17-M58/SEQ ID NO: 222; L chain, L11/SEQ ID NO: 236). Each variant was produced and purified by the method described in Example 4.

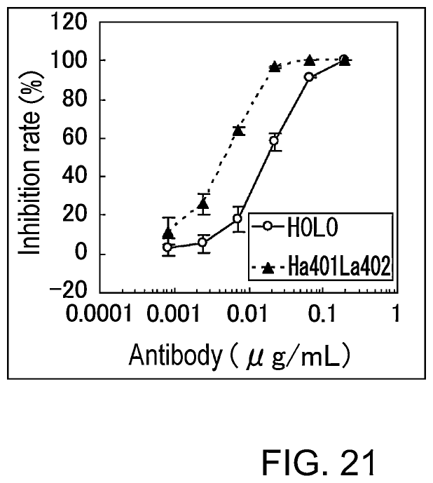

Ha401La402 (H chain, Ha401-SKSC/SEQ ID NO: 255; L chain, La402/SEQ ID NO: 256) was assessed for its affinity for NR10 and its biological activity by the method described in Referential Example 10 and the method using BaF/NR10 as described in Example 2, respectively, and they were compared to those of H0L0 (H chain, H0-SKSC/SEQ ID NO: 54; L chain, L0/SEQ ID NO: 56). The result of affinity measurement is shown in Table 10, and the biological activity determined using BaF/NR10 is shown in FIG. 21. Both affinity and biological activity were found to be improved as compared to those of H0L0 (H chain, H0-SKSC/SEQ ID NO: 54; L chain, L0/SEQ ID NO: 56).

TABLE 10

| | ka(1/Ms) | kd(1/s) | KD(M) |
|---|---|---|---|
| H0L0 | 2.9E+05 | 9.1E−04 | 3.2E−09 |
| Ha401La402 | 5.8E+05 | 2.9E−04 | 5.0E−10 |

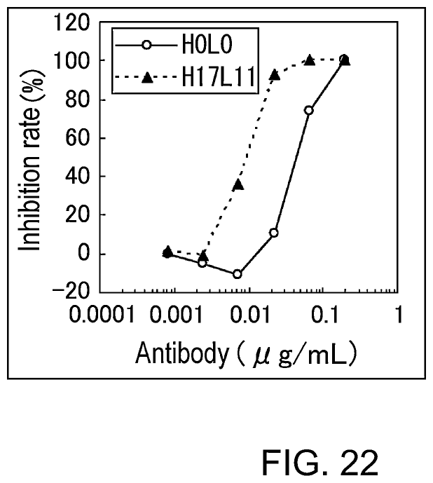

Furthermore, H17L11 (H chain, H17-M58/SEQ ID NO: 222; L chain, L11/SEQ ID NO: 236) was assessed for its affinity for NR10 and its biological activity by the method described in Example 7 and the method using BaF/NR10 as described in Example 2, respectively, and they were compared to those of H0L0 (H chain, H0-M58/SEQ ID NO: 136; L chain, L0/SEQ ID NO: 56). The result of affinity measurement is shown in Table 11, and the biological activity determined using BaF/NR10 is shown in FIG. 22. Both affinity and biological activity were found to be improved as compared to those of H0L0 (H chain, H0-M58/SEQ ID NO: 136; L chain, L0/SEQ ID NO: 56).

TABLE 11

| | ka(1/Ms) | kd(1/s) | KD(M) |
|---|---|---|---|
| H0L0 | 1.4E+05 | 6.9E−04 | 4.8E−09 |
| H17L11 | 4.3E+05 | 2.6E−04 | 6.2E−10 |

### Example 11

### Identification of Mutation Sites Reducing Immunogenicity Risk Reduction of Immunogenicity Risk in H Chain CDR1

T-cell epitopes present in the variable region sequence of H0L0 were analyzed using TEPITOPE (Methods 2004 December; 34(4): 468-75). As a result, CDR1 of the H chain was predicted to have many T-cell epitopes that bind to HLA (i.e. have sequences with a high immunogenicity risk). Then, TEPITOPE analysis was carried out to examine substitutions that would reduce the immunogenicity risk of the H chain CDR1. As a result, the immunogenicity risk was found to be greatly reduced by substituting isoleucine at position 33 in kabat numbering with alanine (A) (Table 12). Then, this alteration was added to H17 generated in Example 10 to produce H19 (H19-M58/SEQ ID NO: 223). The generated H19 was combined with L12 to produce H19L12 (H chain, H19-M58/SEQ ID NO: 223; L chain, L12/SEQ ID NO: 237). Each variant was produced and purified by the method described in Example 4.

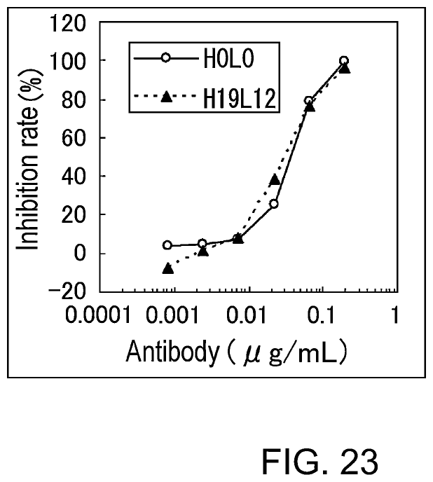

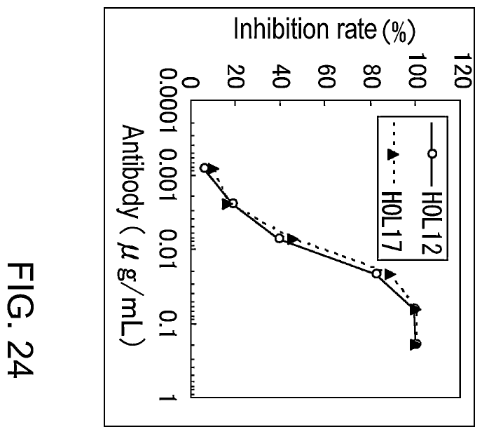

The antibody was assessed for the affinity for NR10 and the biological activity by the method described in Referential Example 10 and the method using BaF/NR10 as described in Example 2, respectively, and they were compared to those of H0L0 (H chain, H0-M58/SEQ ID NO: 136; L chain, L0/SEQ ID NO: 56). The result of affinity measurement is shown in Table 13, and the biological activity determined using BaF/NR10 is shown in FIG. 23. Both affinity and biological activity were shown to be almost equal to those of H0L0.

TABLE 12

| | | | Mutation | site H0 | Amino acid | Sequence |
|---|---|---|---|---|---|---|
| Name | Type | H0 sequence | (kabat No) | sequence | after mutation | after mutation |
| H19 | CDR1 | GYIMN | 33 | I | A | GYAMN |
| | | (SEQ ID NO: 270) | | | | (SEQ ID NO: 284) |

TABLE 13

| | ka(1/Ms) | kd(1/s) | KD(M) |
|---|---|---|---|
| H0L0 | 1.8E+05 | 8.7E−04 | 4.8E−09 |
| H19L12 | 2.3E+05 | 1.2E−03 | 5.1E−09 |

### Reduction of Immunogenicity Risk in L Chain CDR1

Threonine (T) present at kabat-numbering position 25 in CDR1 of the L chain corresponds to alanine (A) or serine (S) in the germline sequence. Thus, it is predicted that the immunogenicity risk is reduced by substituting threonine (T) at position 25 with alanine (A) or serine (S) (Table 14). Therefore, the above substitution was added to L12 to produce L17 (SEQ ID NO: 238). The produced L17 was combined with H0 to produce H0L17 (H chain, H0-M58/SEQ ID NO: 136; L chain, L17/SEQ ID NO: 238). Each variant was produced and purified by the method described in Example 4.

Each variant was assessed for the affinity for NR10 and the biological activity by the method described in Referential Example 10 and the method using BaF/NR10 as described in Example 2, respectively, and they were compared to those of H0L0 (H chain, H0-M58/SEQ ID NO: 136; L chain, L0/SEQ ID NO: 56) and H0L12 (H chain, H0-M58/SEQ ID NO: 136; L chain, L12/SEQ ID NO: 237). Since L12 contains a sequence that improves the affinity, it exhibits about two times higher affinity than H0L0. The result of affinity measurement is shown in Table 15, and the biological activity determined using BaF/NR10 is shown in FIG. 24. Both affinity and biological activity were shown to be almost equal to those of H0L12.

TABLE 14

| | | | Mutation | site L0 | Amino acid | Sequence |
|---|---|---|---|---|---|---|
| Name | Type | L0 sequence | (kabat No) | sequence | after mutation | after mutation |
| Ld-1 | CDR1 | RTSENIYSFLA | 25 | T | A | RASENIYSFLA |
| | | (SEQ ID NO: 277) | | | | (SEQ ID NO: 285) |
| Ld-2 | CDR1 | RTSENIYSFLA | 25 | T | S | RSSENIYSFLA |
| | | (SEQ ID NO: 277) | | | | (SEQ ID NO: 286) |

TABLE 15

| | ka(1/Ms) | kd(1/s) | KD(M) |
|---|---|---|---|
| H0L0 | 1.6E+05 | 7.8E−04 | 4.8E−09 |
| H0L12 | 3.8E+05 | 7.4E−04 | 2.0E−09 |
| H0L17 | 3.9E+05 | 8.1E−04 | 2.1E−09 |

### Example 12

### Preparation of Completely Humanized NS22 Antibody

Variable regions of NS22 variants were prepared by combining the multiple mutations that reduce the pI, increase the affinity, suppress the degradation of H chain, and reduce the immunogenicity risk, all of which were found in the above Examples, in H0 (H0-M58/SEQ ID NO: 136), H1 (H1-M58/SEQ ID NO: 257), or L0 (L0/SEQ ID NO: 56), and subjected to various screening procedures. As a result, H28L17 (H chain, H28-M58/SEQ ID NO: 224; L chain, L17/SEQ ID NO: 238), H30L17 (H chain, H30-M58/SEQ ID NO: 225; L chain, L17/SEQ ID NO: 238), H34L17 (H chain, H34-M58/SEQ ID NO: 226, L chain, L17/SEQ ID NO: 238), H42L17 (H chain, H42-M58/SEQ ID NO: 227; L chain, L17/SEQ ID NO: 238), H44L17 (H chain, H44-M58/SEQ ID NO: 228; L chain, L17/SEQ ID NO: 238), H46L17 (H chain, H46-M58/SEQ ID NO: 229; L chain, L17/SEQ ID NO: 238), H57L17 (H chain, H57-M58/SEQ ID NO: 230; L chain, L17/SEQ ID NO: 238), H71L17 (H chain, H71-M58/SEQ ID NO: 231; L chain, L17/SEQ ID NO: 238), H78L17 (H chain, H78-M58/SEQ ID NO: 232; L chain, L17/SEQ ID NO: 238), H92L17 (H chain, H92-M58/SEQ ID NO: 233; L chain, L17/SEQ ID NO: 238), H97L50 (H chain, H97-M58/SEQ ID NO: 234; L chain, L50/SEQ ID NO: 239), and H98L50 (H chain, H98-M58/SEQ ID NO: 235; L chain, L50/SEQ ID NO: 239) were found. Each variant was produced and purified by the method described in Example 4.

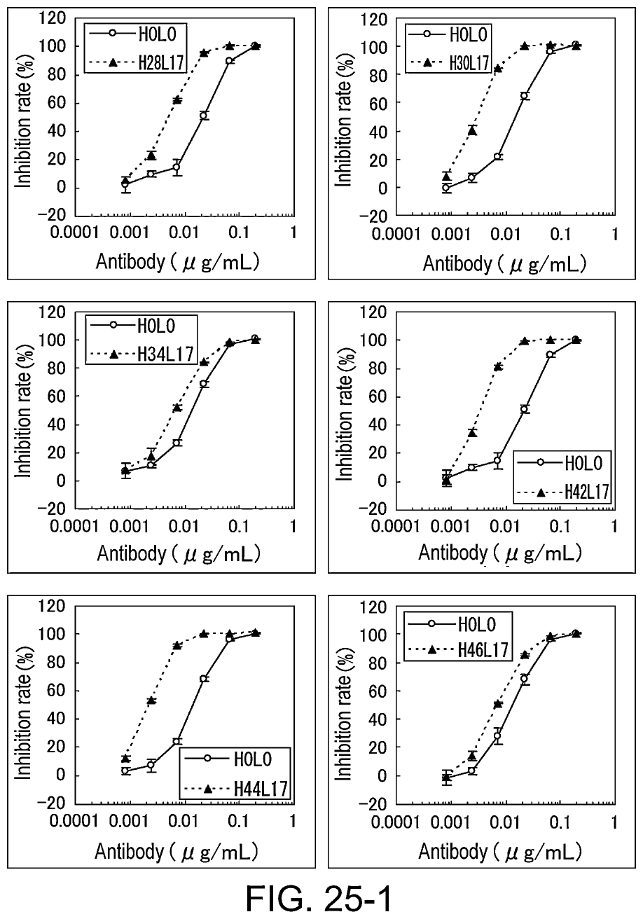

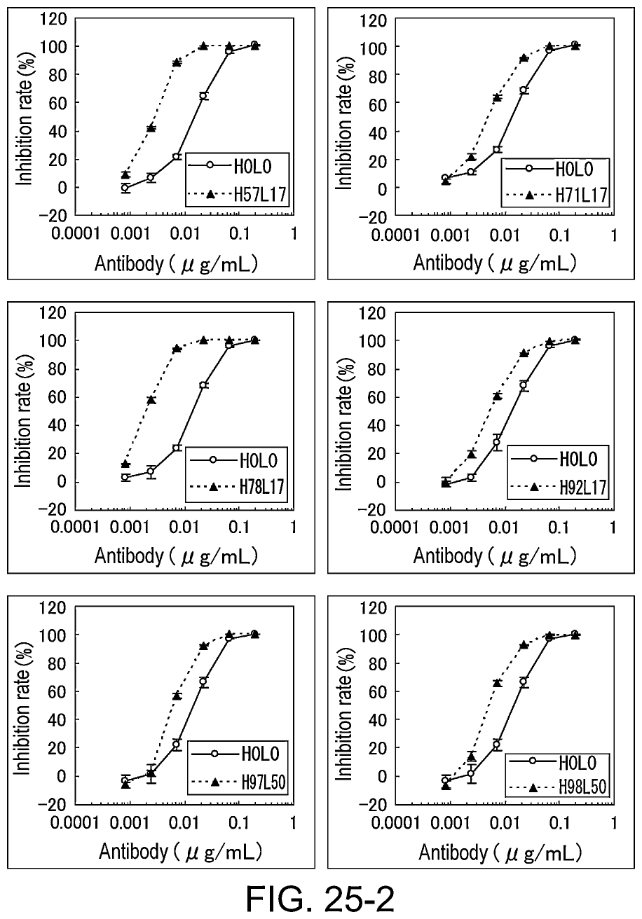

Each variant was assessed for the affinity for NR10 and the biological activity by the method described in Referential Example 10 and the method using BaF/NR10 as described in Example 2, respectively, and they were compared to those of H0L0 (H chain, H0-M58/SEQ ID NO: 136; L chain, L0/SEQ ID NO: 56). The result of affinity measurement is shown in Table 16, and the biological activity determined using BaF/NR10 is shown in FIGS. 25-1 and 25-2. Both affinity and biological activity of each antibody were shown to be almost equal to or greater than those of H0L0.

TABLE 16

| Sample | ka(1/Ms) | kd(1/s) | KD(M) |
|---|---|---|---|
| H0L0 | 2.1E+05 | 8.8E−04 | 4.2E−09 |
| H28L17 | 6.4E+05 | 3.3E−04 | 5.2E−10 |
| H30L17 | 6.8E+05 | 5.7E−04 | 8.3E−10 |
| H34L17 | 3.4E+05 | 1.2E−03 | 3.6E−09 |
| H42L17 | 5.7E+05 | 3.7E−04 | 6.5E−10 |
| H44L17 | 6.1E+05 | 7.2E−04 | 1.2E−09 |
| H46L17 | 2.9E+05 | 1.3E−03 | 4.6E−09 |
| H57L17 | 7.1E+05 | 5.5E−04 | 7.7E−10 |
| H71L17 | 3.7E+05 | 1.2E−03 | 3.3E−09 |
| H78L17 | 6.1E+05 | 7.0E−04 | 1.1E−09 |
| H92L17 | 3.1E+05 | 1.3E−03 | 4.1E−09 |
| H97L50 | 3.6E+05 | 1.3E−03 | 3.5E−09 |
| H98L50 | 2.9E+05 | 1.3E−03 | 4.6E−09 |

### Example 13

### Analysis of the Binding Domain of Anti-NR10 Neutralizing Antibody

### (1) Preparation of Human/Mouse Wild-Type and Chimeric Antigens

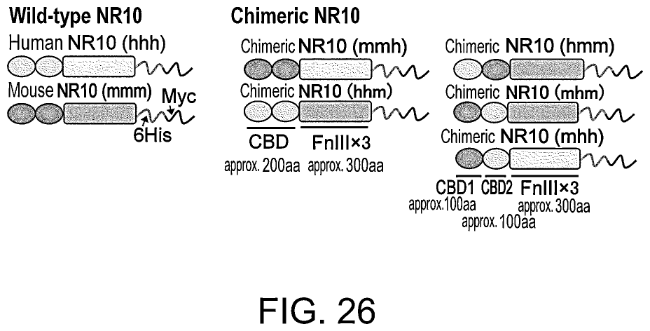

The genes encoding human and mouse wild-type extracellular domains and chimeric extracellular domains of NR10 (hhh (SEQ ID NO: 258), mmm (SEQ ID NO: 259), hhm (SEQ ID NO: 260), mmh (SEQ ID NO: 261), hmm (SEQ ID NO: 262), mhm (SEQ ID NO: 263), and mhh (SEQ ID NO: 264)), were fused to His tag and Myc tag (HHHHHHEQKLISEEDL/SEQ ID NO: 287) at their C termini, inserted into an animal expression vector, and transiently expressed using FreeStyle 293 Expression System (Invitrogen™). Schematic diagrams for the human/mouse wild-type and chimeric NR10-ECDs are shown in FIG. 26.

The human/mouse wild-type and chimeric antigens (hhh, mmm, hhm, mmh, hmm, mhm, and mhh) were purified from culture supernatants by Ni-NTA Superflow column chromatography. Specifically, 1 ml of Ni-NTA Superflow (QIAGEN) was loaded onto Poly-Prep Empty Column (BioRad), and 30 ml of each culture supernatant was added thereto. After washing with D-PBS (Dulbecco's phosphate-buffered saline) containing 150 mM sodium chloride and 20 mM imidazole, the column was eluted with D-PBS containing 150 mM sodium chloride and 250 mM imidazole. The eluted fractions were buffer-exchanged with D-PBS and concentrated using Amicon-Ultra (Millipore) with a molecular weight cut-off of 10K.

### (2) Detection of Binding Antigen by Western Blotting

Each of the prepared human/mouse wild-type and chimeric antigens was electrophoresed at 0.5 □g/lane on three 4-20% polyacrylamide gels (Daiichi Pure Chemicals Co.). The proteins were electro-transferred onto PVDF membranes (Millipore) in a semi-dry blotting apparatus, and the membranes were blocked with TBS containing 5% skim milk. One membrane was incubated with 5 □g/ml of H44M58L17 (detection system for humanized anti-human NR 10 antibody); another with 5 □g/ml of ND41 (detection system for mouse anti-human NR 10 antibody); and the other one with anti-Myc antibody (SantaCruz, Cat. #sc-789) 500-times diluted with TBS containing 5% skim milk (detection system for Myc tag) at room temperature for one hour.

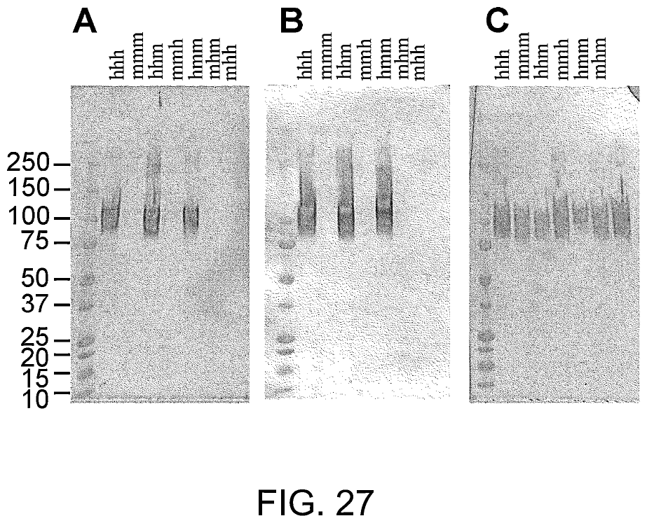

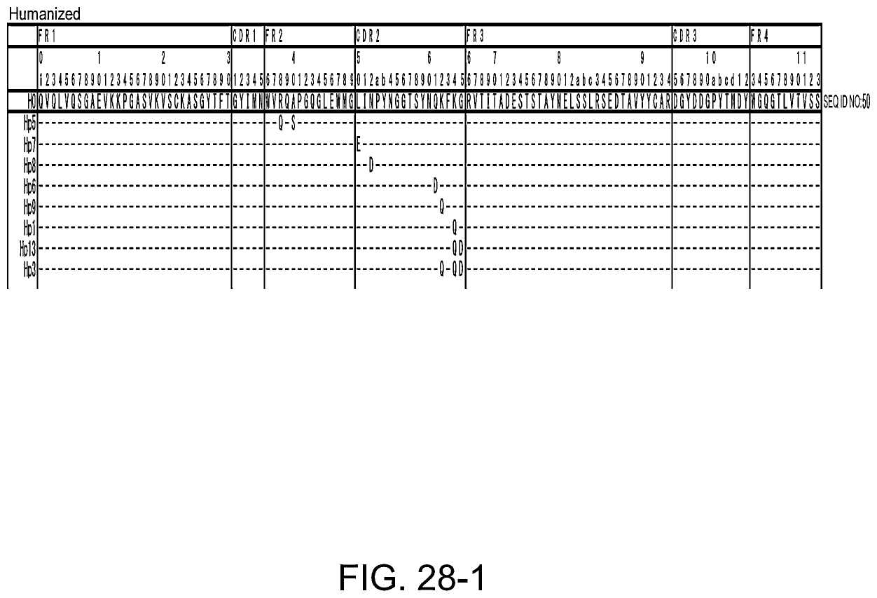

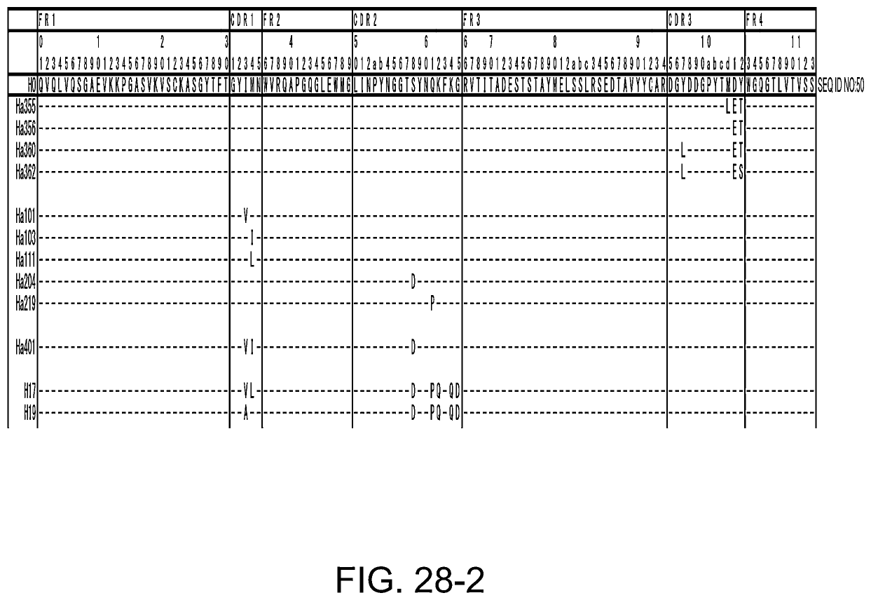

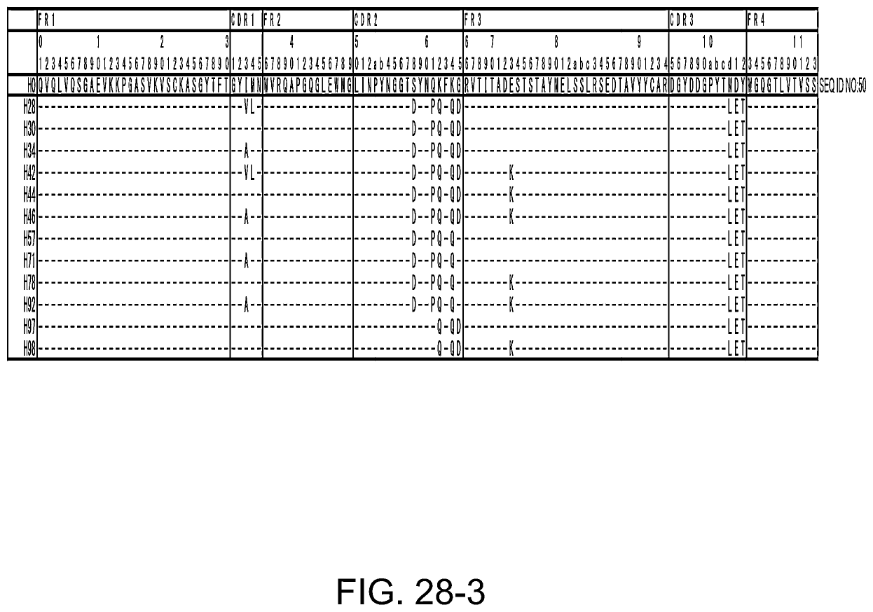

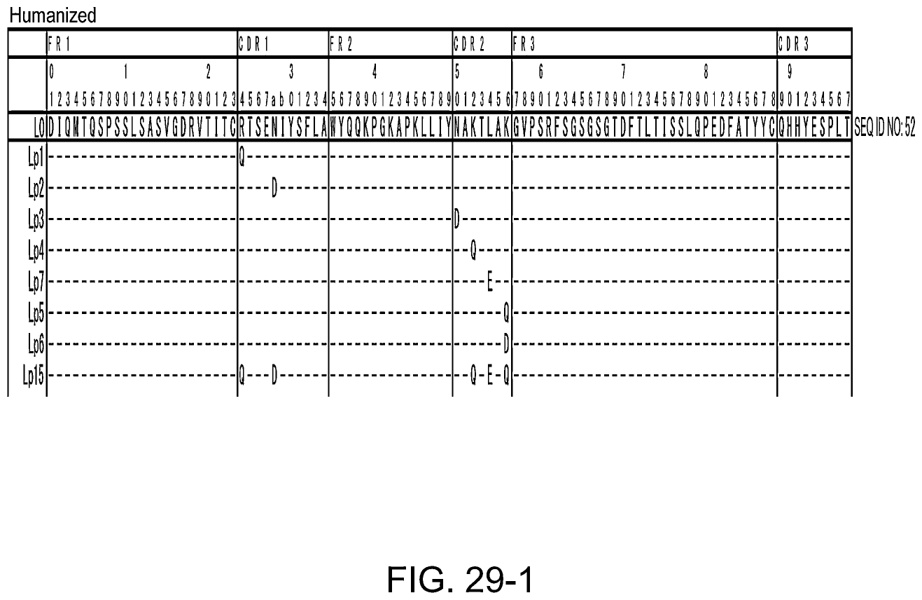

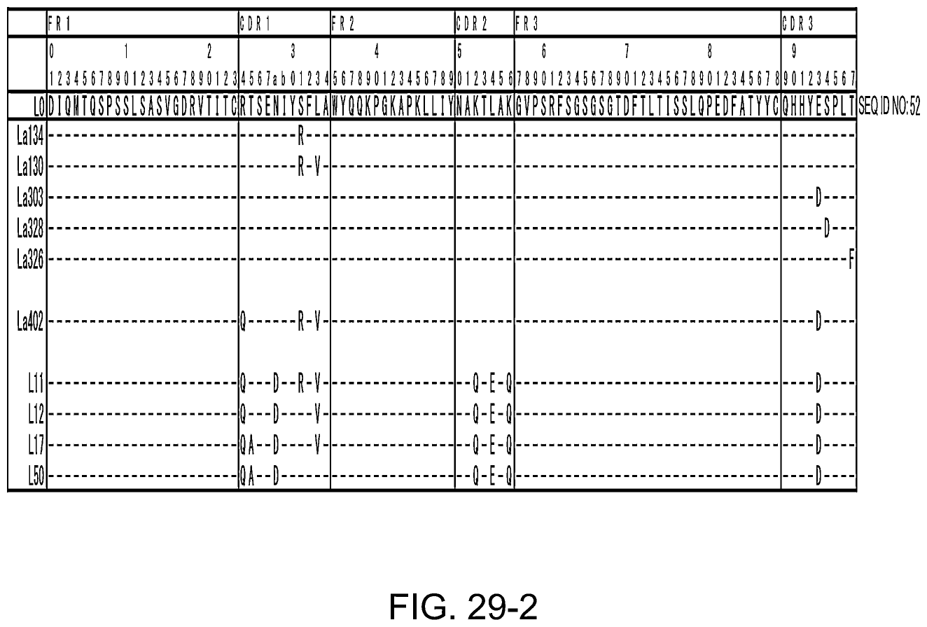

After washing three times with TBS containing 0.05% Tween™ 20, the secondary antibodies were incubated with the membranes. Alkaline phosphatase-labeled goat anti-human IgG (BIOSOURCE, Cat. #AHI0305) was used to detect humanized anti-human NR10 antibody; alkaline phosphatase-labeled goat anti-mouse IgG (SantaCruz, Cat. #sc-2008) was used to detect mouse anti-human NR10 antibody; and alkaline phosphatase-labeled goat anti-rabbit IgG (SantaCruz, Cat. #sc-2057) was used to detect Myc tag. The reaction was carried out at room temperature for one hour. After washing four times with TBS containing 0.05% Tween™ 20 for three minutes, color development was carried out using BCIP/NBT Phosphatase substrate, 1-Component System (KPL). TBS (Tris-buffered saline) used here was prepared by dissolving a pack of TBS (Tris buffered saline) powder (TaKaRa) in 1 L of distilled water. The result is shown in FIG. 27.

When the humanized antibody or mouse antibody was used, the binding was detected only for hhh, hhm, and hmm, which are NR 10 extracellular domains.

### Referential Example 1

### Isolation of Cynomolgus Monkey NR10, OSMR, and IL-31 Genes

Since the cross-reactivity and neutralizing activity in cynomolgus monkeys were considered important for safety assessment at a pre-clinical stage, the cynomolgus monkey NR10 and OSMR genes were isolated. Primers were designed based on published information of Rhesus monkey genome and others, and the NR10 and OSMR genes were successfully amplified by PCR from cynomolgus monkey pancreatic cDNA. The sequences of the isolated cynomolgus monkey NR10, OSMR, and IL-31 genes are shown in SEQ ID NOs: 65, 69, and 67, respectively, and the amino acid sequences of cynomolgus monkey NR10, OSMR, and IL-31 are shown in SEQ ID NOs: 66, 70, and 68, respectively.

### Referential Example 2

### Establishment of NR10- and OSMR-Expressing Ba/F3 Cell Lines

The full-length human NR10 cDNA (SEQ ID NO: 75) was inserted into the expression vector pCOS1 (Biochem. Biophys. Res. Commun. 228, p838-45, 1996), and the resulting vector was named pCosNR10.3. An oncostatin M receptor cDNA (OSMR, GenBank accession No. NM003999) was isolated by PCR from a human placental library, and the expression vector pCos1-hOSMR was constructed in the same manner. 10 □g each of the vectors were simultaneously introduced into mouse IL-3-dependent pro-B cell-derived cell line Ba/F3 by electroporation (BioRad Gene Pulser, 960 □F, 0.33 kV). After introduction, human IL-31 (R&D Systems) was added, and the cells were cultured to obtain a cell line (hNR10/hOSMR/BaF3 cell) that proliferates in an IL-31-dependent manner. Furthermore, the cynomolgus monkey IL-31 gene (SEQ ID NO: 67) was inserted into a mammalian cell expression vector and introduced into CHO cell line DG44. The resulting culture supernatant was obtained as cynomolgus monkey IL-31. As with hNR10/hOSMR/BaF3, the full-length cynomolgus monkey NR10 and OSMR genes were inserted into the expression vector pCOS1 and expressed in Ba/F3 cells, and a cynomolgus monkey IL-31-dependent cell line (cynNR10/cynOSMR/BaF3 cell) was established using the culture supernatant described above.

### Referential Example 3

### Establishment of NR10-Expressing CHO Cell Lines

The genes for cytoplasmic domain-lacking human NR10 (SEQ ID NO: 73) and cytoplasmic domain-lacking cynomolgus monkey NR10 (SEQ ID NO: 71) were each inserted to a mammalian cell expression vector. The resulting vectors were linearized with a restriction enzyme, and then introduced into CHO cell line DG44 by electroporation (BioRad Gene Pulser, 25 □F, 1.5 kV). After drug selection, NR10-expressing cells were selected and established by FCM analysis using anti-human NR10 antibody. The amino acid sequence encoded by the nucleotide sequence of cytoplasmic domain-lacking human NR10 gene (SEQ ID NO: 73) is shown in SEQ ID NO: 74, and the amino acid sequence encoded by the nucleotide sequence of cytoplasmic domain-lacking cynomolgus monkey NR10 gene (SEQ ID NO: 71) is shown in SEQ ID NO: 72.

### Referential Example 4

### Preparation of NR10 Protein (Extracellular Domain)

The human NR10 cDNA was used as a template to amplify only the extracellular domain by PCR. The amplified region was then fused to a FLAG tag sequence at the C terminus and inserted to a mammalian cell expression vector. Ten □g of the linearized vector was introduced into Chinese hamster ovary cell line DG44 by electroporation (BioRad Gene PulserII, 25 □F, 1.5 kV). A cell line showing high level expression was obtained. The supernatant of the cell line cultured on a large scale was purified using anti-FLAG antibody column (Sigma) and gel filtration to obtain soluble NR10. The nucleotide sequence of soluble NR 10 is shown in SEQ ID NO: 77, and the amino acid sequence is shown in SEQ ID NO: 78.

### Referential Example 5

### Preparation of Anti-Human NR10 Antibodies

Mice were immunized with human NR10 protein (extracellular domain) (described in Referential Example 4), and hybridomas were prepared by a conventional method. The culture supernatants of these hybridomas were assessed for the neutralizing activity using the human IL-31-dependent cell line (hNR10/hOSMR/BaF3 cell) described in Referential Example 2, and thereby NA633 which has an NR10-neuralizing activity was obtained.

Furthermore, DNA immunization was carried out by He gas-driven gene gun using a mammalian expression vector carrying the full-length human NR10 gene (SEQ ID NO: 75), and hybridomas were prepared by a conventional method. The culture supernatants of these hybridomas were assessed for the neutralizing activity using the human IL-31-dependent cell line (hNR10/hOSMR/BaF3 cell) described in Referential Example 2, and thereby ND41 which has an NR10-neuralizing activity was obtained.

### Referential Example 6

### Preparation of Human Chimeric antibody

The amino acid sequences of heavy chain and light chain variable regions of NA633 are shown in SEQ ID NOs: 104 and 108, respectively. The amino acid sequences of CDR1, CDR2, and CDR3 of the heavy chain variable region of NA633 are shown in SEQ ID NOs: 105, 106, and 107, respectively, while those of CDR1, CDR2, and CDR3 of the light chain variable region are shown in SEQ ID NOs: 109, 110, and 111, respectively. Furthermore, a chimeric antibody between these mouse variable regions and human constant region (H chain, □1; L chain, □) was produced by a conventional method.

### Referential Example 7

### Preparation of huPM1-SKSC in Which the Heterogeneity of Wild Type IgG2 is Reduced without Loss of Stability

Since the NS22 antibody is an NR10-neutralizing antibody, its binding to Fc receptor may be unfavorable in consideration of the immunogenicity and adverse effects. A possible method for reducing the binding to Fc□ receptor is to select IgG2 or IgG4 instead of IgG1 as the isotype of the constant region (Ann Hematol. 1998 June; 76(6): 231-48.). From the viewpoint of Fc□0 receptor I and retention in plasma, IgG2 has been considered more desirable than IgG4 (Nat Biotechnol. 2007 December; 25(12): 1369-72). Meanwhile, when an antibody is developed as a pharmaceutical, properties of the protein, particularly homogeneity and stability, are highly important. The IgG2 isotype has been reported to have very high heterogeneity resulting from the disulfide bonds in the hinge region (J Biol Chem. 2008 Jun. 6; 283(23): 16206-15.). It is not easy and would be more costly to manufacture it as pharmaceutical in a large scale while maintaining difference in the heterogeneity of desired/related substances among products resulting from the above. Accordingly, it is desired that the substance be composed of a single substance as much as possible. Thus, when antibodies of IgG2 isotype are developed as pharmaceuticals, it is preferred to reduce the heterogeneity resulting from disulfide bonds, without lowering the stability.

In order to reduce the heterogeneity of the wild type IgG2, cysteines in the hinge region and CH1 domain of IgG2 were substituted. As a result of examination of various variants, SKSC (SEQ ID NO: 62), which is a constant region obtained by altering cysteine at position 131 and arginine at position 133 in the EU numbering (Sequences of proteins of immunological interest, NIH Publication No.91-3242) within the H-chain CH1 domain of the wild type IgG2 constant region sequence to serine and lysine, respectively, and altering cysteine at EU-numbering position 219 in the H-chain upper hinge to serine could reduce the heterogeneity without decreasing the stability. Meanwhile, other possible methods for decreasing heterogeneity are to alter only cysteine at EU-numbering position 219 in the H-chain upper hinge to serine, and to alter only cysteine at EU-numbering position 220 to serine. Thus, constant region SC (SEQ ID NO: 153) in which cysteine at EU-numbering position 219 in IgG2 has been altered to serine, and constant region CS (SEQ ID NO: 154) in which cysteine at EU-numbering position 220 in IgG2 has been altered to serine, were produced.

huPMI-SC (SEQ ID NO: 157), huPM1-CS (SEQ ID NO: 158), huPM1-IgG1 (SEQ ID NO: 159), huPM1-IgG2 (SEQ ID NO: 160), and huPM1-SKSC (SEQ ID NO: 161), which were prepared by combining the constant regions produced as above, IgG1 (SEQ ID NO: 60), and IgG2 (SEQ ID NO: 132) with the variable region of the humanized anti-IL-6 receptor antibody (H chain variable region, huPM1-VH/SEQ ID NO: 155; L chain variable region huPM1-VL/SEQ ID NO: 156)(Cancer Res. 1993 Feb. 15;53(4): 851-6.), were used as an H chain, and huPM1-L (SEQ ID NO: 162) was used as an L chain, to produce each antibody. Each antibody was expressed and purified by the method described in Example 4.

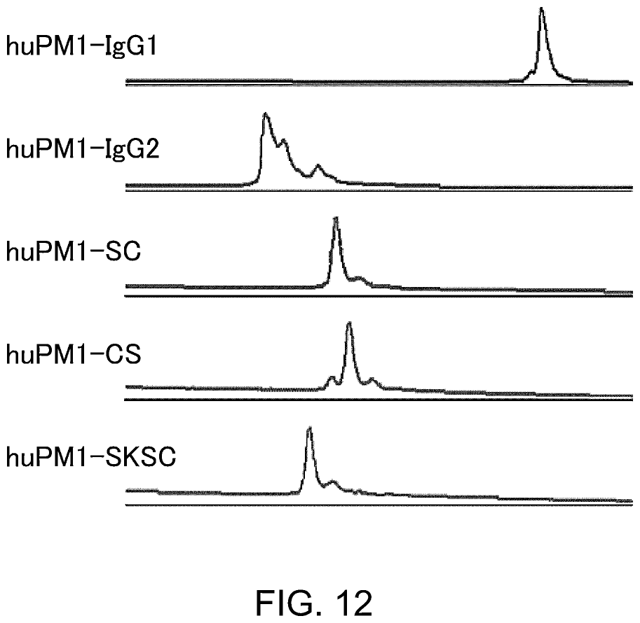

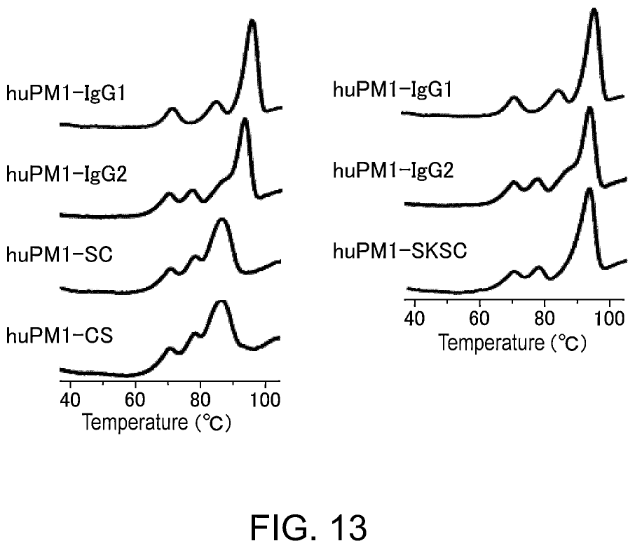

The antibodies were compared to each other in terms of the heterogeneity. The heterogencity of huPM1-IgG1, huPM1-IgG2, huPM1-SC, huPM1-CS, and huPM1-SKSC was assessed by cation exchange chromatography. The chromatography was carried out using a ProPac WCX-10 (Dionex) column, 20 mM sodium acetate (pH 5.0) as mobile phase A, and 20 mM sodium acetate/1M NaCl (pH 5.0) as mobile phase B, with an appropriate flow rate and gradient. The result of assessment by cation exchange chromatography is shown in FIG. 12.

As shown in FIG. 12, conversion of the constant region from IgG1 into IgG2 increased the heterogeneity. In contrast, the heterogeneity was markedly reduced by converting the constant region into SKSC. While constant region SC resulted in considerable reduction of the heterogeneity as in SKSC, constant region CS did not sufficiently improve the heterogeneity. When an antibody is developed as a pharmaceutical, it is generally desired that the antibody have high stability in addition to low heterogeneity for the production of stable preparations. Thus, to assess the stability, the thermal denaturation midpoint temperature (Tm value) was determined by differential scanning calorimetry (DSC) (VP-DSC; Microcal). The thermal denaturation midpoint temperature (Tm value) serves as an indicator of stability. In order to prepare stable preparations as pharmaceuticals, a higher thermal denaturation midpoint temperature (Tm value) is preferred (J Pharm Sci. 2008 April; 97(4): 1414-26.). Thus, huPM1-IgG1, huPM1-IgG2, huPM1-SC, huPM1-CS, and huPM1-SKSC were dialyzed against a solution of 20 mM sodium acetate/150 mM NaCl (pH 6.0) (EasySEP; TOMY), and DSC measurement was carried out using about 0.1 mg/ml of protein at a heating rate of 1° C./min between 40 and 100° C. The denaturation curves obtained by DSC are shown in FIG. 13. The Tm values of the Fab domains are listed in Table 17 below.

TABLE 17

| Name | Tm/° C. |
|---|---|
| huPM1-IgG1 | 94.8 |
| huPM1-IgG2 | 93.9 |
| huPM1-SC | 86.7 |
| huPM1-CS | 86.4 |
| huPM1-SKSC | 93.7 |

The Tm values of huPM1-IgG1 and huPM1-IgG2 were almost the same, namely, about 94° C. (IgG2 was lower by about 1° C.). Meanwhile, the Tm values of huPM1-SC and huPM1-CS were about 86° C., which was significantly lower than those of huPM1-IgG1 and huPM1-IgG2. On the other hand, the Tm value of huPMI-SKSC was about 94° C., and almost the same as huPM1-IgG1 and huPM1-IgG2. Since the stability of huPM1-SC and huPM1-CS was markedly lower than that of IgG2, huPM1-SKSC in which cysteine in the CH1 domain have also been altered to serine may be more preferred in the development of pharmaceuticals. The significant decrease in Tm value of huPM1-SC and huPM1-CS as compared to IgG2 may be due to the disulfide-bonding pattern of huPMI-SC and huPM1-CS that is different from that of IgG2.

Furthermore, comparison of the DSC denaturation curves showed that the denaturation peak for the Fab domain was sharp in huPM1-IgG1 and huPM1-SKSC, while it was broader in huPM1-SC and huPM1-CS than the above two, and huPM1-IgG2 gave a shoulder peak on the lower temperature side of the Fab domain denaturation peak. The denaturation peak in DSC generally becomes sharp in the case of a single component, but may become broad when two or more components with different Tm values (namely, heterogeneity) are present. Thus, it was suggested that huPM1-IgG2, huPM1-SC, and huPM1-CS contained two or more components, and the heterogeneity of natural IgG2 was not reduced in huPM1-SC and huPMI-CS. This finding suggests that cysteines present in both the hinge region and the CH1 domain are involved in the heterogencity of natural IgG2, and it is necessary to alter not only cysteine in the hinge region but also that in the CH1 domain to decrease the heterogeneity on DSC. Furthermore, as described above, it is only possible to attain stability equivalent to that of natural IgG2 by altering not only cysteine in the hinge region but also that in the CH1 domain.

As described above, as to the constant regions in which the heterogeneity resulting from the hinge region of IgG2 has been reduced, it was discovered that SC and CS, which are constant regions in which only cysteine in the hinge region has been substituted with serine, may be insufficient from the viewpoint of heterogencity and stability, and that it is only possible to significantly reduce the heterogencity while maintaining the stability comparable to IgG2 by additionally substituting cysteine at EU-numbering position 131 in the CH1 domain with serine. Such constant regions include SKSC.

### Referential Example 8

### Production and Assessment of Optimized, Non-Fc Receptor-Binding Constant Region M14

In the Fc receptor-binding domain of IgG2 constant region, the residues at EU-numbering positions 233, 234, 235, and 236 are of non-binding type, while the residues at EU-numbering positions 327, 330, and 331 are different from those of IgG4, which are of non-binding type. Thus, it is necessary to alter the amino acids at EU-numbering positions 327, 330, and 331 to the sequence of IgG4 (G2Aa in Eur J Immunol. 1999 August; 29(8):2613-24). However, since the amino acid at EU-numbering position 339 is alanine in IgG4 while it is threonine in IgG2, mere alteration of the amino acids at EU-numbering positions 327, 330, and 331 to the sequence of IgG4 will generate a novel non-naturally occurring 9-amino acid peptide sequence that could be a T-cell epitope peptide, thereby causing a risk of immunogenicity. Thus, it was found that the occurrence of the novel peptide sequence could be prevented by altering threonine at EU-numbering position 339 in IgG2 to alanine, in addition to the alterations described above. In addition to the mutations described above, methionine at EU-numbering position 397 was mutated into valine to improve the stability of IgG2 under acidic condition. Furthermore, in SKSC (SEQ ID NO: 62) produced in Referential Example 7, in which the heterogeneity resulting from the disulfide bonds in the hinge region has been improved, introduction of mutations at positions 131 and 133 will generate a novel non-naturally occurring 9-amino acid peptide sequence that could be a T-cell epitope peptide, thereby causing a risk of immunogenicity. Thus, the peptide sequence around positions 131 to 139 was converted into the same as IgG1 by mutating glutamic acid at EU-numbering position 137 into glycine and mutating serine at EU-numbering position 138 into glycine. The constant region sequence M14 (SEQ ID NO: 129) was produced by introducing all the above mutations.

The expression and purification of huPM1-M14, prepared by using huPM1-M14 as an H chain and huPM1-L (SEQ ID NO: 162) as an L chain, was carried out by the method described in Referential Example 7. The prepared huPM1-M14 (SEQ ID NO: 163), huPM1-IgG1, and huPM1-IgG2 were assessed for the heterogencity using cation exchange chromatography by the method described in Referential Example 7.

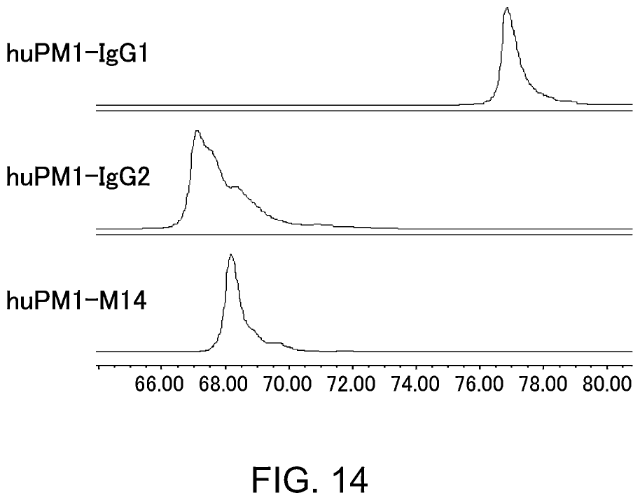

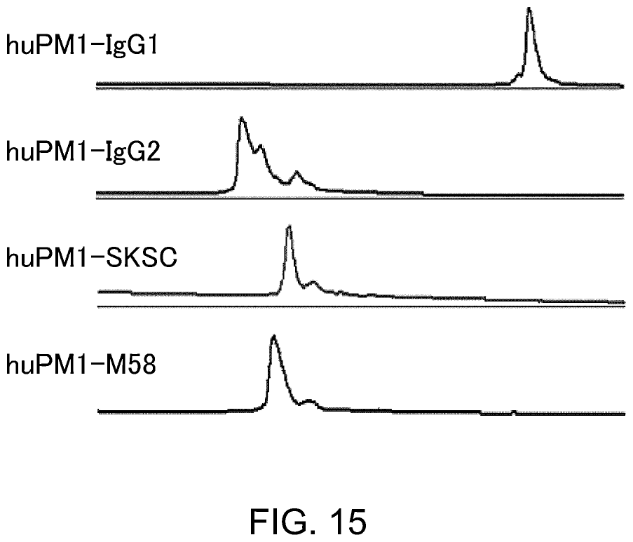

As shown in FIG. 14, the heterogeneity was also reduced in huPM1-M14 as in huPM1-SKSC.

### Referential Example 9

### Preparation of huPM1-M58 with Reduced H-Chain C-terminal Heterogeneity and Improved Pharmacokinetics

Preparation of huPM1-M58 Molecule

huPM1 is an IgG1 antibody. For the heterogeneity in the C-terminal sequence of the H chain of IgG antibody, the deletion of the C-terminal lysine residue and the amidation of the C-terminal amino group due to deletion of the two C-terminal amino acids, glycine and lysine, have been reported (Anal Biochem. 2007 Jan. 1;360(1): 75-83). Also in huPM1, while the major component is a sequence in which the C-terminal lysine encoded by the nucleotide sequence has been deleted by post-translational modification, there are also a minor component in which the lysine remains and a minor component in which the C-terminal amino group is amidated due to deletion of both glycine and lysine, which contribute to heterogeneity. Producing a pharmaceutical in a large scale while maintaining the difference in the heterogencity of desired/related substances between products is not easy but rather results in increase of cost, and it is thus desired that the substance be composed of a single substance as much as possible. When an antibody is developed as a pharmaceutical, reduction of the heterogeneity is desired. Thus, it is desired that the C-terminal of the H chain has no heterogeneity when developed as pharmaceuticals. It is also desirable to prolong the plasma half-life of the antibody in order to reduce the antibody dose.

Thus, the alterations described below were introduced to prepare a novel constant region in which the heterogeneity at C-terminal of the H chain has been reduced, the pharmacokinetics has been improved as compared to huPM1-IgG1, and the heterogeneity derived from wild-type IgG2 has also been reduced without loss of stability.

Specifically, in huPM1-SKSC, which has high stability and in which the above-mentioned heterogeneity related to antibodies with IgG2-isotype constant regions is reduced, glutamic acid at EU-numbering position 137 was substituted with glycine; serine at position 138 with glycine; histidine at position 268 with glutamine; arginine at position 355 with glutamine; and glutamine at position 419 with glutamic acid. In addition to the above substitutions, glycine and lysine at positions 446 and 447 were deleted to reduce the heterogeneity of the H-chain C terminus, thereby obtaining huPM1-M58 (SEQ ID NO: 164). huPM1-M58 prepared by using huPM1-M58 as an H chain and huPM1-L (SEQ ID NO: 162) as an L chain was expressed and purified by the method described in Example 4.

The huPM1-M58, huPM1-IgG1, and huPM1-IgG2 were assessed for the heterogeneity and stability by the methods described in Example 5 using cation exchange chromatography and DSC, respectively.

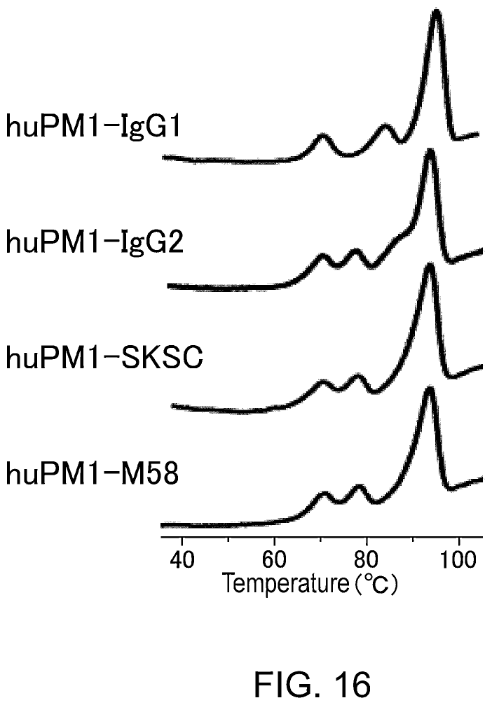

The result of DSC is shown in Table 18. As shown in FIGS. 13 and 16, huPM1-M58 was found to show reduced heterogeneity without loss of stability as in huPM1-SKSC.

TABLE 18

| Name | Tm/° C. |
|---|---|
| huPM1-IgG1 | 94.8 |
| huPM1-IgG2 | 93.9 |
| huPM1-SKSC | 93.7 |
| huPM1-M58 | 93.7 |

Assessment of huPM1-M58 for Plasma Retention

The prolonged retention (slow elimination) of IgG molecule in plasma is due to the function of FcRn, which is known as a salvage receptor of IgG molecule (Nat Rev Immunol. 2007 September; 7(9): 715-25). When incorporated into endosomes via pinocytosis, IgG molecules bind to FcRn expressed in endosomes under the acidic conditions within the endosome (approx. pH 6.0). While IgG molecules that are not bound to FcRn are transferred to and degraded in lysosomes, those bound to FcRn are translocated to the cell surface and then released from FcRn into plasma again under the neutral conditions in plasma (approx. pH 7.4). IgG-type antibodies are known to include IgG1, IgG2, IgG3, and IgG4 isotypes. The plasma half-lives of these isotypes in human are reported to be about 36 days for IgG1 and IgG2; about 29 days for IgG3; and 16 days for IgG4 (Nat. Biotechnol. 2007 December; 25(12): 1369-72). Thus, the retention of IgG1 and IgG2 in plasma is believed to be the longest. In general, the isotypes of antibodies used as pharmaceutical agents are IgG1, IgG2, and IgG4. Reported methods for further improving the pharmacokinetics of these IgG antibodies include methods for improving the above-described binding activity to human FcRn by altering the sequence of IgG constant region (J. Biol. Chem. 2007 Jan. 19;282(3): 1709-17; J. Immunol. 2006 Jan. 1;176(1): 346-56).

There are species differences between mouse FcRn and human FcRn (Proc. Natl. Acad. Sci. USA. 2006 Dec. 5;103(49): 18709-14). Therefore, to predict the retention of IgG antibodies having an altered constant region sequence in human plasma, it may be desirable to assess the binding to human FcRn and the plasma retention in human FcRn transgenic mice (Int. Immunol. 2006 December;18(12): 1759-69).

### Assessment of the Binding to Human FcRn

FcRn is a complex of FcRn and 2-microglobulin. Oligo-DNA primers were prepared based on the published human FcRn gene sequence (J. Exp. Med. (1994) 180 (6), 2377-2381). A DNA fragment encoding the whole gene was prepared by PCR using human cDNA (Human Placenta Marathon-Ready cDNA, Clontech) as a template and the prepared primers. Using the obtained DNA fragment as a template, a DNA fragment encoding the extracellular domain containing the signal region (Metl-Leu290) was amplified by PCR, and inserted into an animal cell expression vector (the amino acid sequence of human FcRn/SEQ ID NO: 165). Likewise, oligo-DNA primers were prepared based on the published human 2-microglobulin gene sequence (Proc. Natl. Acad. Sci. U.S.A. 99 (26), 16899-16903 (2002)). A DNA fragment encoding the whole gene was prepared by PCR using human cDNA (Hu-Placenta Marathon-Ready cDNA, CLONTECH) as a template and the prepared primers. Using the obtained DNA fragment as a template, a DNA fragment encoding the whole □2-microglobulin containing the signal region (Met1-Met119) was amplified by PCR and inserted into an animal cell expression vector (the amino acid sequence of human □2-microglobulin/SEQ ID NO: 166).

Soluble human FcRn was expressed by the following procedure. The prepared plasmids for human FcRn and □2-microglobulin were introduced into the human embryonic kidney cancer-derived cell line HEK293H (Invitrogen) using 10% fetal bovine serum

(Invitrogen) by lipofection. The resulting culture supernatant was collected and purified using IgG Sepharose 6 Fast Flow (Amersham Biosciences) by the method described in J. Immunol. 2002 Nov. 1; 169(9):5171-80. Then further purification was carried out using HiTrap Q HP (GE Healthcare).

The binding to human FcRn was assessed using Biacore 3000. An antibody was bound to Protein L or rabbit anti-human IgG Kappa chain antibody immobilized onto a sensor chip, human FcRn was added as an analyte for interaction with the antibody, and the affinity (KD) was calculated from the amount of bound human FcRn. Specifically, Protein L was immobilized onto sensor chip CM5 (BIACORE) by the amine coupling method using 50 mM Na-phosphate buffer (pH 6.0) containing 150 mM NaCl as the running buffer. Then, an antibody was diluted with the running buffer containing 0.02% Tween20, and injected and allowed to bind to the chip. Human FcRn was then injected to assess the binding activity of the antibody to the human FcRn.

The affinity was calculated using BIAcvaluation software. The obtained sensorgram was used to calculate the amount of hFcRn bound to the antibody immediately before the end of human FcRn injection. This was fitted by the steady state affinity method to calculate the affinity of human FcRn for the antibody.

Predictive Assessment of Plasma Retention of huPM1-IgG1 and huPM1-M58 in Human Using Human FcRn

The binding activities of huPM1-IgG1 and huPM1-M58 to human FcRn were assessed using BIAcore. As shown in Table 19, the binding activity of huPM1-M58 was greater than that of huPM1-IgG1 by about 1.4 times.

TABLE 19

| | KD (μM) |
|---|---|
| huPM1-IgG1 | 1.62 |
| huPM1-M58 | 1.17 |

### Assessment of the Plasma Retention in Human FcRn Transgenic Mice

The pharmacokinetics in human FcRn transgenic mice (B6.mFcRn−/−. hFcRn Tg line 276 +/+ mice; Jackson Laboratories) was assessed by the following procedure. An antibody was intravenously administered once at a dose of 1 mg/kg to mice, and blood was collected at appropriate time points. The collected blood was immediately centrifuged at 15,000 rpm for 15 minutes at 4° C. to obtain plasma. The separated plasma was stored in a freezer at −20° C. or below until use. The plasma concentration was determined by ELISA.

Predictive Assessment of the Plasma Retention of huPM1-IgG1 and huPM1-M58 in human using Human FcRn Transgenic Mice

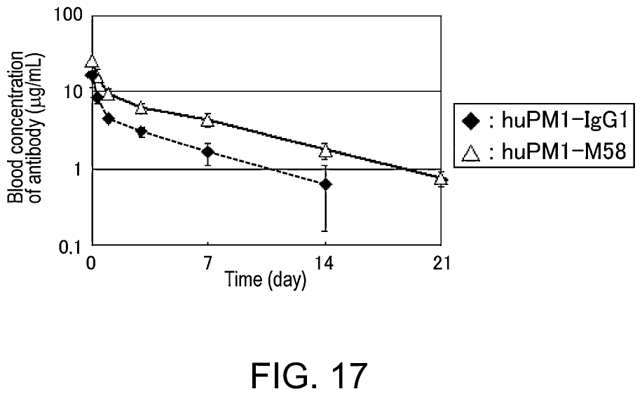

The plasma retention of huPM1-IgG1 and huPM1-M58 in human FcRn transgenic mice was assessed. As shown in FIG. 17, the result demonstrated that the pharmacokinetics of huPM1-M58 was improved as compared to huPM1-IgG1. It was suggested that the human FcRn-binding activity was correlated to the plasma retention in human FcRn transgenic mice.

### Referential Example 10

### Measurement of the Affinity in Antigen-Antibody Reaction using Biacore

Kinetic analysis of the aantigen-antibody reaction was carried out using Biacore T100 (GE Healthcare Biosciences). The antigen-antibody interaction was measured by immobilizing rec-Protein A (hereinafter Protein A) (ZYMED) onto a sensor chip, capturing an antibody on the immobilized Protein A, and then reacting the antigen as an analyte. Various concentrations of rhNR10 were used as the antigen. The kinetic parameters, association rate constant kₐ (1/Ms) and dissociation rate constant k_{d} (1/s), were calculated from the sensorgrams obtained by the measurement. Then, K_{D} (M) was determined based on the rate constants. Each parameter was determined using Biacore T100 Evaluation Software version 1.1 (GE Healthcare Biosciences).

Immobilization of Protein A onto Sensor Chip

Protein A was immobilized onto all flow cells of sensor chip CM5 (GE Healthcare Bioscinences) by the amine coupling method. The experiment was carried out using HBS-EP+ (10 mM HEPES, 0.15 M NaCl, 3 mM EDTA, 0.05% v/v Surfactant P20) as a running buffer at a flow rate of 10 □L/min. The carboxyl groups of carboxymethyl dextran on the sensor chip were activated with 100 □L of a 1:1 mixture of 75 mg/ml EDC (N-ethyl-N′-(3-dimethylaminopropyl)carbodiimide hydrochloride) and 11.5 mg/ml NHS (N-hydroxysuccinimide), and Protein A prepared at 50 □g/ml using 10 mM acetate buffer (pH 4.5) was allowed to flow for reaction. Then, 100 □L of 1 M ethanolamine hydrochloride (pH 8.5) was allowed to flow to inactivate the unreacted active groups. Ultimately, about 4000 to 5000 RU were immobilized. The experiment was carried out at 25° C. at all times.

Measurement of affinity in antigen-antibody reaction between rhNR10 and antibody captured on Protein A

The running buffer used was HBS-EP+. Each antibody was prepared at 0.25 g/ml, or prepared so that about 100 RU would bind to Protein A. rhNR10 used as an analyte was prepared at 0, 38.5, 77.0, and 154 nM, or at 0, 19.25, and 77.01 nM using HBS-EP+. In the measurement, first, the antibody solution was captured on Protein A, and an analyte solution was reacted at a flow rate of 20 □L/min for three minutes. Then, the solution was switched to HBS-EP+, and the dissociation phase was measured for five minutes. After measurement of the dissociation phase, the sensor chip was regenerated by washing with 10 mM glycine-HCl (pH 1.5). The obtained sensorgrams were kinetically analyzed using the Biacore-specific data analysis software, Biacore T100 Evaluation Software Version 1.1.

### INDUSTRIAL APPLICABILITY

The anti-NR10 antibodies obtained by the present inventors exhibit an effective neutralizing activity against NR10, and are useful as, for example, therapeutic agents for inflammatory diseases.

1. An antibody that recognizes domain 1 of NR10.

2. The antibody of claim 1, which has a neutralizing activity.

3. The antibody of claim 1 or 2, which is a humanized antibody.

4. An anti-NR10 antibody which is any one of:
(1) an antibody comprising a heavy chain variable region which comprises CDR1 comprising the amino acid sequence of SEQ ID NO: 1, CDR2 comprising the amino acid sequence of SEQ ID NO: 2, and CDR3 comprising the amino acid sequence of SEQ ID NO: 3;
(2) an antibody comprising the heavy chain variable region of SEQ ID NO: 4;
(3) an antibody comprising a light chain variable region which comprises CDR1 comprising the amino acid sequence of SEQ ID NO: 5, CDR2 comprising the amino acid sequence of SEQ ID NO: 6, and CDR3 comprising the amino acid sequence of SEQ ID NO: 7;
(4) an antibody comprising the light chain variable region of SEQ ID NO: 8;
(5) an antibody comprising the heavy chain variable region of (1) and the light chain variable region of (3);
(6) an antibody comprising the heavy chain variable region of (2) and the light chain variable region of (4);
(7) an antibody in which one or more amino acids are substituted, deleted, added, and/or inserted in the antibody of any one of (1) to (6), which has an activity equivalent to that of the antibody of any one of (1) to (6); and
(8) an antibody which binds to the same epitope as an epitope bound by the antibody of any one of (1) to (7).

5. An anti-NR10 antibody which is any one of:
(1) an antibody comprising a heavy chain variable region which comprises CDR1 comprising the amino acid sequence of SEQ ID NO: 9, CDR2 comprising the amino acid sequence of SEQ ID NO: 10, and CDR3 comprising the amino acid sequence of SEQ ID NO: 11;
(2) an antibody comprising the heavy chain variable region of SEQ ID NO: 12;
(3) an antibody comprising a light chain variable region which comprises CDR1 comprising the amino acid sequence of SEQ ID NO: 13, CDR2 comprising the amino acid sequence of SEQ ID NO: 14, and CDR3 comprising the amino acid sequence of SEQ ID NO: 15;
(4) an antibody comprising the light chain variable region of SEQ ID NO: 16;
(5) an antibody comprising the heavy chain variable region of (1) and the light chain variable region of (3);
(6) an antibody comprising the heavy chain variable region of (2) and the light chain variable region of (4);
(7) an antibody in which one or more amino acids are substituted, deleted, added, and/or inserted in the antibody of any one of (1) to (6), which has an activity equivalent to that of the antibody of any one of (1) to (6); and
(8) an antibody which binds to the same epitope as an epitope bound by the antibody of any one of (1) to (7).

6. An anti-NR10 antibody which is any one of:
(1) an antibody comprising a heavy chain variable region which comprises CDR1 comprising the amino acid sequence of SEQ ID NO: 17, CDR2 comprising the amino acid sequence of SEQ ID NO: 18, and CDR3 comprising the amino acid sequence of SEQ ID NO: 19;
(2) an antibody comprising the heavy chain variable region of SEQ ID NO: 20;
(3) an antibody comprising a light chain variable region which comprises CDR1 comprising the amino acid sequence of SEQ ID NO: 21, CDR2 comprising the amino acid sequence of SEQ ID NO: 22, and CDR3 comprising the amino acid sequence of SEQ ID NO: 23;
(4) an antibody comprising the light chain variable region of SEQ ID NO: 24;
(5) an antibody comprising the heavy chain variable region of (1) and the light chain variable region of (3);
(6) an antibody comprising the heavy chain variable region of (2) and the light chain variable region of (4);
(7) an antibody in which one or more amino acids are substituted, deleted, added, and/or inserted in the antibody of any one of (1) to (6), which has an activity equivalent to that of the antibody of any one of (1) to (6); and
(8) an antibody which binds to the same epitope as an epitope bound by the antibody of any one of (1) to (7).

7. An anti-NR10 antibody which is any one of:
(1) an antibody comprising a heavy chain variable region which comprises CDR1 comprising the amino acid sequence of SEQ ID NO: 25, CDR2 comprising the amino acid sequence of SEQ ID NO: 26, and CDR3 comprising the amino acid sequence of SEQ ID NO: 27;
(2) an antibody comprising the heavy chain variable region of SEQ ID NO: 28;
(3) an antibody comprising a light chain variable region which comprises CDR1 comprising the amino acid sequence of SEQ ID NO: 29, CDR2 comprising the amino acid sequence of SEQ ID NO: 30, and CDR3 comprising the amino acid sequence of SEQ ID NO: 31;
(4) an antibody comprising the light chain variable region of SEQ ID NO: 32;
(5) an antibody comprising the heavy chain variable region of (1) and the light chain variable region of (3);
(6) an antibody comprising the heavy chain variable region of (2) and the light chain variable region of (4);
(7) an antibody in which one or more amino acids are substituted, deleted, added, and/or inserted in the antibody of any one of (1) to (6), which has an activity equivalent to that of the antibody of any one of (1) to (6); and
(8) an antibody which binds to the same epitope as an epitope bound by the antibody of any one of (1) to (7).

8. An antibody or antibody variable region which is any one of:
(1) a heavy chain variable region comprising CDR1 of SEQ ID NO: 196, CDR2 of SEQ ID NO:
197, and CDR3 of SEQ ID NO: 11 (H17);
(2) a heavy chain variable region comprising CDR1 of SEQ ID NO: 176, CDR2 of SEQ ID NO:
197, and CDR3 of SEQ ID NO: 11 (H19);
(3) a heavy chain variable region comprising CDR1 of SEQ ID NO: 196, CDR2 of SEQ ID NO:
197, and CDR3 of SEQ ID NO: 184 (H28, H42);
(4) a heavy chain variable region comprising CDR1 of SEQ ID NO: 9, CDR2 of SEQ ID NO:
197, and CDR3 of SEQ ID NO: 184 (H30, H44);
(5) a heavy chain variable region comprising CDR1 of SEQ ID NO: 176, CDR2 of SEQ ID NO:
197, CDR3 of SEQ ID NO: 184 (H34, H46);
(6) a heavy chain variable region comprising CDR1 of SEQ ID NO: 9, CDR2 of SEQ ID NO:
198, and CDR3 of SEQ ID NO: 184 (H57, H78);
(7) a heavy chain variable region comprising CDR1 of SEQ ID NO: 176, CDR2 of SEQ ID NO:
198, and CDR3 of SEQ ID NO: 184 (H71, H92);
(8) a heavy chain variable region comprising CDR1 of SEQ ID NO: 9, CDR2 of SEQ ID NO:
199, and CDR3 of SEQ ID NO: 184 (H97, H98);
(9) a light chain variable region comprising CDR1 of SEQ ID NO: 200, CDR2 of SEQ ID NO:
170, and CDR3 of SEQ ID NO: 193 (L11);
(10) a light chain variable region comprising CDR1 of SEQ ID NO: 201, CDR2 of SEQ ID NO:
170, and CDR3 of SEQ ID NO: 193 (L12);
(11) a light chain variable region comprising CDR1 of SEQ ID NO: 202, CDR2 of SEQ ID NO:
170, and CDR3 of SEQ ID NO: 193 (L17);
(12) a light chain variable region comprising CDR1 of SEQ ID NO: 203, CDR2 of SEQ ID NO:
170, and CDR3 of SEQ ID NO: 193 (L50);
(13) an antibody comprising the heavy chain variable region of (3) and the light chain variable region of (11);
(14) an antibody comprising the heavy chain variable region of (4) and the light chain variable region of (11);
(15) an antibody comprising the heavy chain variable region of (5) and the light chain variable region of (11);
(16) an antibody comprising the heavy chain variable region of (6) and the light chain variable region of (11);
(17) an antibody comprising the heavy chain variable region of (7) and the light chain variable region of (11);
(18) an antibody comprising the heavy chain variable region of (8) and the light chain variable region of (12);
(19) an antibody in which one or more amino acids are substituted, deleted, added, and/or inserted in the antibody of any one of (13) to (18), which has an activity equivalent to that of the antibody of any one of (13) to (18); and
(20) an antibody which binds to the same epitope as an epitope bound by the antibody of any one of (13) to (18).

9. An antibody or antibody variable region which is any one of:
(1) a heavy chain variable region comprising the amino acid sequence of SEQ ID NO: 204 (H17);
(2) a heavy chain variable region comprising the amino acid sequence of SEQ ID NO: 205 (H19);
(3) a heavy chain variable region comprising the amino acid sequence of SEQ ID NO: 206 (H28);
(4) a heavy chain variable region comprising the amino acid sequence of SEQ ID NO: 207 (H30);
(5) a heavy chain variable region comprising the amino acid sequence of SEQ ID NO: 208 (H34), (6) a heavy chain variable region comprising the amino acid sequence of SEQ ID NO: 209 (H42);
(7) a heavy chain variable region comprising the amino acid sequence of SEQ ID NO: 210 (H44);
(8) a heavy chain variable region comprising the amino acid sequence of SEQ ID NO: 211 (H46);
(9) a heavy chain variable region comprising the amino acid sequence of SEQ ID NO: 212 (H57);
(10) a heavy chain variable region comprising the amino acid sequence of SEQ ID NO: 213 (H71);
(11) a heavy chain variable region comprising the amino acid sequence of SEQ ID NO: 214 (H78);
(12) a heavy chain variable region comprising the amino acid sequence of SEQ ID NO: 215 (H92);
(13) a heavy chain variable region comprising the amino acid sequence of SEQ ID NO: 216 (H97);
(14) a heavy chain variable region comprising the amino acid sequence of SEQ ID NO: 217 (H98);
(15) a light chain variable region comprising the amino acid sequence of SEQ ID NO: 218 (L11);
(16) a light chain variable region comprising the amino acid sequence of SEQ ID NO: 219 (L12);
(17) a light chain variable region comprising the amino acid sequence of SEQ ID NO: 220 (L17);
(18) a light chain variable region comprising the amino acid sequence of SEQ ID NO: 221 (L50);
(19) an antibody comprising the heavy chain variable region of (3) and the light chain variable region of (17) (H28L17);
(20) an antibody comprising the heavy chain variable region of (4) and the light chain variable region of (17) (H30L17);
(21) an antibody comprising the heavy chain variable region of (5) and the light chain variable region of (17) (H34L17);
(22) an antibody comprising the heavy chain variable region of (6) and the light chain variable region of (17) (H42L17);
(23) an antibody comprising the heavy chain variable region of (7) and the light chain variable region of (17) (H44L17);
(24) an antibody comprising the heavy chain variable region of (8) and the light chain variable region of (17) (H46L17);
(25) an antibody comprising the heavy chain variable region of (9) and the light chain variable region of (17) (H57L17);
(26) an antibody comprising the heavy chain variable region of (10) and the light chain variable region of (17) (H71L17);
(27) an antibody comprising the heavy chain variable region of (11) and the light chain variable region of (17) (H78L17);
(28) an antibody comprising the heavy chain variable region of (12) and the light chain variable region of (17) (H92L17);
(29) an antibody comprising the heavy chain variable region of (13) and the light chain variable region of (18) (H97L50);
(30) an antibody comprising the heavy chain variable region of (14) and the light chain variable region of (18) (H98L50), (31) an antibody in which one or more amino acids are substituted, deleted, added, and/or inserted in the antibody of any one of (19) to (30), which has an activity equivalent to that of the antibody of any one of (19) to (30); and
(32) an antibody which binds to the same epitope as an epitope bound by the antibody of any one of (19) to (30).

10. The anti-NR10 antibody of any one of claims 4 to 9, which is a humanized antibody.

11. An antibody, antibody heavy chain, or antibody light chain, which is any one of:
(1) a heavy chain comprising the amino acid sequence of SEQ ID NO: 222 (H17);
(2) a heavy chain comprising the amino acid sequence of SEQ ID NO: 223 (H19);
(3) a heavy chain comprising the amino acid sequence of SEQ ID NO: 224 (H28);
(4) a heavy chain comprising the amino acid sequence of SEQ ID NO: 225 (H30);
(5) a heavy chain comprising the amino acid sequence of SEQ ID NO: 226 (H34);
(6) a heavy chain comprising the amino acid sequence of SEQ ID NO: 227 (H42);
(7) a heavy chain comprising the amino acid sequence of SEQ ID NO: 228 (H44);
(8) a heavy chain comprising the amino acid sequence of SEQ ID NO: 229 (H46);
(9) a heavy chain comprising the amino acid sequence of SEQ ID NO: 230 (H57);
(10) a heavy chain comprising the amino acid sequence of SEQ ID NO: 231 (H71);
(11) a heavy chain comprising the amino acid sequence of SEQ ID NO: 232 (H78);
(12) a heavy chain comprising the amino acid sequence of SEQ ID NO: 233 (H92);
(13) a heavy chain comprising the amino acid sequence of SEQ ID NO: 234 (H97);
(14) a heavy chain comprising the amino acid sequence of SEQ ID NO: 235 (H98);
(15) a light chain comprising the amino acid sequence of SEQ ID NO: 236 (L11);
(16) a light chain comprising the amino acid sequence of SEQ ID NO: 237 (L12);
(17) a light chain comprising the amino acid sequence of SEQ ID NO: 238 (L17);
(18) a light chain comprising the amino acid sequence of SEQ ID NO: 239 (L50);
(19) an antibody comprising the heavy chain of (3) and the light chain of (17) (H28L17);
(20) an antibody comprising the heavy chain of (4) and the light chain of (17) (H30L17);
(21) an antibody comprising the heavy chain of (5) and the light chain of (17) (H34L17);
(22) an antibody comprising the heavy chain of (6) and the light chain of (17) (H42L17);
(23) an antibody comprising the heavy chain of (7) and the light chain of (17) (H44L17);
(24) an antibody comprising the heavy chain of (8) and the light chain of (17) (H46L17);
(25) an antibody comprising the heavy chain of (9) and the light chain of (17) (H57L17);
(26) an antibody comprising the heavy chain of (10) and the light chain of (17) (H71L17);
(27) an antibody comprising the heavy chain of (11) and the light chain of (17) (H78L17);
(28) an antibody comprising the heavy chain of (12) and the light chain of (17) (H92L17);
(29) an antibody comprising the heavy chain of (13) and the light chain of (18) (H97L50);
(30) an antibody comprising the heavy chain of (14) and the light chain of (18) (H98L50);
(31) an antibody in which one or more amino acids are substituted, deleted, added, and/or inserted in the antibody of any one of (19) to (30), which has an activity equivalent to that of the antibody of any one of (19) to (30); and
(32) an antibody which binds to the same epitope as an epitope bound by the antibody of any one of (19) to (30).

12. A pharmaceutical composition comprising the antibody of any one of claims 1 to 11.

13. The pharmaceutical composition of claim 12, which is an agent for treating an inflammatory disease.


SEQUENCE LISTING (287 sequences)

SEQ ID NO: 1 - protein, 5 aa, Mus musculus
GYIMN

SEQ ID NO: 2 - protein, 17 aa, Mus musculus
LINPYNGDTNYNQKFKG

SEQ ID NO: 3 - protein, 12 aa, Mus musculus
DGYDDGPYTMDY

SEQ ID NO: 4 - protein, 121 aa, Mus musculus
EVQLQQSGPELVKPGASMKISCKASGYSFTGYIMNWVKQSHGKNLEWIGLINPYNGDTNYNQKFKGKATLTVDKSSSTAYMELLSLTSEDSAVYYCARDGYDDGPYTMDYWGQGTSVTVSS

SEQ ID NO: 5 - protein, 11 aa, Mus musculus
RASENIYSFLA

SEQ ID NO: 6 - protein, 7 aa, Mus musculus
NAKTLAK

SEQ ID NO: 7 - protein, 9 aa, Mus musculus
QHHYESPLT

SEQ ID NO: 8 - protein, 107 aa, Mus musculus
DIQMTQSPASLSASVGETVTITCRASENIYSFLAWYQQKQGKSPHLLVYNAKTLAKGVPSRFSGSGSGTQFSLKINSLQPEDFGSYYCQHHYESPLTFGGGTKLEIK

SEQ ID NO: 9 - protein, 5 aa, Mus musculus
GYIMN

SEQ ID NO: 10 - protein, 17 aa, Mus musculus
LINPYNGGTSYNQKFKG

SEQ ID NO: 11 - protein, 12 aa, Mus musculus
DGYDDGPYTMDY

SEQ ID NO: 12 - protein, 121 aa, Mus musculus
EVQLQQSGPELVKPGASMKISCKASGYSFTGYIMNWVKQSHGKNLEWIGLINPYNGGTSYNQKFKGKATLTVDKSSSTAYMELLSLTSEDSAVYYCARDGYDDGPYTMDYWGQGTSVTVSS

SEQ ID NO: 13 - protein, 11 aa, Mus musculus
RTSENIYSFLA

SEQ ID NO: 14 - protein, 7 aa, Mus musculus
NAKTLAK

SEQ ID NO: 15 - protein, 9 aa, Mus musculus
QHHYESPLT

SEQ ID NO: 16 - protein, 107 aa, Mus musculus
DIQMTQSPASLSASVGETVTITCRTSENIYSFLAWYQQKQGKSPHLLVYNAKTLAKGVPSRFSGSGSGTQFSLKINSLQPEDFGSYFCQHHYESPLTFGGGTKLEIK

SEQ ID NO: 17 - protein, 5 aa, Mus musculus
GYIMN

SEQ ID NO: 18 - protein, 17 aa, Mus musculus
LINPYNGGAEYNQKFKD

SEQ ID NO: 19 - protein, 12 aa, Mus musculus
DGYDDGPYTMDY

SEQ ID NO: 20 - protein, 121 aa, Mus musculus
EVQLQQSGPELVKPGTSMKISCKASGYSFTGYIMNWVKQSHGKNLEWIGLINPYNGGAEYNQKFKDKATFTVDKSSSTAYMELLSLTSEDSAVYYCARDGYDDGPYTMDYWGQGTSVTVSS

SEQ ID NO: 21 - protein, 11 aa, Mus musculus
RANENIYSYLA

SEQ ID NO: 22 - protein, 7 aa, Mus musculus
NAKTLAE

SEQ ID NO: 23 - protein, 9 aa, Mus musculus
QHHYGTPPT

SEQ ID NO: 24 - protein, 107 aa, Mus musculus
DIQMTQSPASLSASVGETVTFTCRANENIYSYLAWYQQKQGKSPQLLVYNAKTLAEGVPSRFSGSGSGTQFSLKINSLQPEDFGSYYCQHHYGTPPTFGGGTKLEIK

SEQ ID NO: 25 - protein, 5 aa, Mus musculus
NYWMH

SEQ ID NO: 26 - protein, 17 aa, Mus musculus
AIYPGNSDTDYNQKFKG

SEQ ID NO: 27 - protein, 8 aa, Mus musculus
DGYDDFDH

SEQ ID NO: 28 - protein, 116 aa, Mus musculus
EVQLQQSGTVLARPGASVKMSCKASGYTFTNYWMHWVKQRPGQGLEWIGAIYPGNSDTDYNQKFKGKAKLTAVTSASTAYMELSSLTNEDSAVFFCTTGYDDFDHWGQGTTLTVSS

SEQ ID NO: 29 - protein, 12 aa, Mus musculus
RASSSVSSSYLH

SEQ ID NO: 30 - protein, 7 aa, Mus musculus
STSNLAS

SEQ ID NO: 31 - protein, 9 aa, Mus musculus
QQYSGYPLT

SEQ ID NO: 32 - protein, 108 aa, Mus musculus
ENVLTQSPAIMSASPGEKVTMTCRASSSVSSSYLHWYQQKSGASPKLWIYSTSNLASGVPARFSGSGSGTSYYFTISSVEAEDAATYYCQQYSGYPLTFGGGTKLEIK

SEQ ID NO: 33 - DNA, 1406 bp, Mus musculus
  source 1..1406: mol_type = unassigned DNA
atgggatggagctggatctttctcttcctcctgtcaggaactgcaggtgtccactctgaggtccagctgcaacagtctggacctgagctggtgaagcctggagcttcaatgaagatctcctgcaaggcttctggttactcattcactggctacatcatgaactgggtgaagcagagccatggaaagaaccttgagtggattggacttattaatccttacaatggtgatactaactacaaccagaagttcaagggcaaggccacattaactgtagacaagtcatccagcacagcctacatggaactcctcagtctgacatcagaggactctgcagtctattactgtgcaagggatggttacgacgacggaccctatactatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaataatgagcggccgc

SEQ ID NO: 34 - protein, 445 aa, Mus musculus
EVQLQQSGPELVKPGASMKISCKASGYSFTGYIMNWVKQSHGKNLEWIGLINPYNGDTNYNQKFKGKATLTVDKSSSTAYMELLSLTSEDSAVYYCARDGYDDGPYTMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 35 - DNA, 716 bp, Mus musculus
  source 1..716: mol_type = unassigned DNA
atgagtgtgcccactcaggtcctggggttgctgctgctgtggcttacaggtgccagatgtgacatccagatgactcagtctccagcctccctatctgcatctgtgggagaaactgtcaccatcacatgtcgagcaagtgagaatatttacagttttttagcatggtatcagcagaaacagggaaaatctcctcacctcctggtctataatgcaaaaaccttagcaaaaggtgtgccatcaaggttcagtggcagtggatctggcacacagttttctctgaagatcaacagcctgcagcctgaagattttgggagttattactgtcaacatcattatgagagtcctctgacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaatgagcggccgc

SEQ ID NO: 36 - protein, 214 aa, Mus musculus
DIQMTQSPASLSASVGETVTITCRASENIYSFLAWYQQKQGKSPHLLVYNAKTLAKGVPSRFSGSGSGTQFSLKINSLQPEDFGSYYCQHHYESPLTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 37 - DNA, 1406 bp, Mus musculus
  source 1..1406: mol_type = unassigned DNA
atgggatggagctggatctttctcttcctcctgtcaggaactgcaggtgtccactctgaggtccagctgcaacagtctggacctgagctggtgaagcctggagcttcaatgaagatctcctgcaaggcttctggttactcattcactggctacatcatgaactgggtgaagcagagccatggaaagaaccttgagtggattggacttattaatccttacaatggtggtactagctacaaccagaagttcaagggcaaggccacattaactgtagacaagtcatccagtacagcctacatggaactcctcagtctgacatcagaggactctgcagtctattactgtgcaagggatggttacgacgacggaccctatactatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaataatgagcggccgc

SEQ ID NO: 38 - protein, 445 aa, Mus musculus
EVQLQQSGPELVKPGASMKISCKASGYSFTGYIMNWVKQSHGKNLEWIGLINPYNGGTSYNQKFKGKATLTVDKSSSTAYMELLSLTSEDSAVYYCARDGYDDGPYTMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 39 - DNA, 716 bp, Mus musculus
  source 1..716: mol_type = unassigned DNA
atgagtgtgcccactcaggtcctggggttgctgctgctgtggcttacaggtgccagatgtgacatccagatgactcagtctccagcctccctatctgcatctgtgggagaaactgtcaccatcacatgtcgaacaagtgagaatatttacagttttttagcatggtatcagcagaaacagggaaaatctcctcacctcctggtctataatgcaaaaaccttagcaaaaggtgtgccatcaaggttcagtggcagtggatctggcacacagttttctctgaagatcaacagcctgcagcctgaagattttgggagttatttctgtcaacatcattatgagagtcctctgacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaatgagcggccgc

SEQ ID NO: 40 - protein, 214 aa, Mus musculus
DIQMTQSPASLSASVGETVTITCRTSENIYSFLAWYQQKQGKSPHLLVYNAKTLAKGVPSRFSGSGSGTQFSLKINSLQPEDFGSYFCQHHYESPLTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 41 - DNA, 1406 bp, Mus musculus
  source 1..1406: mol_type = unassigned DNA
atgggatggagctggatctttctcttcctcctgtcaggaactgcaggtgtccactctgaggtccagctgcaacagtctggacctgagctggtgaagcctggaacttcaatgaagatatcctgcaaggcttctggttactcattcactggctacatcatgaactgggtgaagcagagccatggaaagaaccttgagtggattggacttattaatccttacaatggtggtgctgagtacaaccagaagttcaaggacaaggccacattcactgtagacaagtcatccagcacagcctacatggagctcctcagtctgacatctgaagactctgcagtctattactgtgcaagggatggttacgacgacggaccctatactatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaataatgagcggccgc

SEQ ID NO: 42 - protein, 445 aa, Mus musculus
EVQLQQSGPELVKPGTSMKISCKASGYSFTGYIMNWVKQSHGKNLEWIGLINPYNGGAEYNQKFKDKATFTVDKSSSTAYMELLSLTSEDSAVYYCARDGYDDGPYTMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 43 - DNA, 716 bp, Mus musculus
  source 1..716: mol_type = unassigned DNA
atgagtgtgcccactcaggtcctggggttgctgctgctgtggcttacaggtgccagatgtgacatccagatgactcagtctccagcctccctatctgcatctgtgggagaaactgtcaccttcacatgtcgagcaaatgagaatatttacagttatttagcatggtatcagcagaaacagggaaaatctcctcagctcctggtctataatgcaaaaaccttagcagaaggtgtgccatcaaggttcagtggcagtggatcaggcacacagttttctctgaagatcaacagcctgcagcctgaagattttgggagttattactgtcaacatcattatggaactcctccgacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaatgagcggccgc

SEQ ID NO: 44 - protein, 214 aa, Mus musculus
DIQMTQSPASLSASVGETVTFTCRANENIYSYLAWYQQKQGKSPQLLVYNAKTLAEGVPSRFSGSGSGTQFSLKINSLQPEDFGSYYCQHHYGTPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 45 - DNA, 1391 bp, Mus musculus
  source 1..1391: mol_type = unassigned DNA
atggaatgtaactggatacttccttttattctgtcggtaatttcaggggtctactcagaggttcagctccagcagtctgggactgtgctggcaaggcctggggcttccgtgaagatgtcctgcaaggcttctggctacacctttaccaactactggatgcactgggtaaaacagaggcctggacagggtctagaatggattggtgctatttatcctggaaatagtgatactgactacaaccagaagttcaagggcaaggccaaactgactgcagtcacatccgccagcactgcctacatggaactcagcagcctgacaaatgaggactctgcggtctttttctgtaccactggttacgacgacttcgaccactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaataatgagcggccgc

SEQ ID NO: 46 - protein, 440 aa, Mus musculus
EVQLQQSGTVLARPGASVKMSCKASGYTFTNYWMHWVKQRPGQGLEWIGAIYPGNSDTDYNQKFKGKAKLTAVTSASTAYMELSSLTNEDSAVFFCTTGYDDFDHWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 47 - DNA, 725 bp, Mus musculus
  source 1..725: mol_type = unassigned DNA
atggattttctggtgcagattttcagcttcttgctaatcagtgcctcagttgcaatgtccagaggagaaaatgtgctcacccagtctccagcaatcatgtctgcatctccaggggaaaaggtcaccatgacctgcagggccagctcaagtgtaagttccagttacttgcactggtaccagcagaagtcaggtgcctcccccaaactctggatttatagcacttccaacttggcttctggagtccctgctcgcttcagtggcagtgggtctgggacctcttactatttcacaatcagcagtgtggaggctgaagatgctgccacttattactgccagcaatacagtggttacccactcacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaatgagcggccgc

SEQ ID NO: 48 - protein, 215 aa, Mus musculus
ENVLTQSPAIMSASPGEKVTMTCRASSSVSSSYLHWYQQKSGASPKLWIYSTSNLASGVPARFSGSGSGTSYYFTISSVEAEDAATYYCQQYSGYPLTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 49 - DNA, 425 bp, synthetic construct
  misc_feature 1..425: note = an artificially synthesized sequence
  source 1..425: mol_type = other DNA
ccaccatggactggacctggagggtcttctgcttgctggctgtagctccaggtgctcactcccaggtgcagctggtgcagtctggggctgaggtgaagaagcctggggcctcagtgaaggtttcctgcaaggcatctggatacaccttcaccggctacatcatgaactgggtgcgacaggcccctggacaagggcttgagtggatgggacttattaatccttacaatggtggtactagctacaaccagaagttcaagggcagagtcacgattaccgcggacgaatccacgagcacagcctacatggagctgagcagcctgagatctgaggacacggccgtgtattactgtgcgagagatggttacgacgacggaccctatactatggactactggggccagggcaccctcgtcacagtctcctca

SEQ ID NO: 50 - protein, 121 aa, synthetic construct
  REGION 1..121: note = an artificially synthesized sequence
QVQLVQSGAEVKKPGASVKVSCKASGYTFTGYIMNWVRQAPGQGLEWMGLINPYNGGTSYNQKFKGRVTITADESTSTAYMELSSLRSEDTAVYYCARDGYDDGPYTMDYWGQGTLVTVSS

SEQ ID NO: 51 - DNA, 398 bp, synthetic construct
  misc_feature 1..398: note = an artificially synthesized sequence
  source 1..398: mol_type = other DNA
ccaccatggacatgagggtccccgctcagctcctggggctcctgctactctggctccgaggtgccagatgtgacatccagatgacccagtctccatcctccctgtctgcatctgtaggagacagagtcaccatcacttgccgaacaagtgagaatatttacagttttttagcatggtatcagcagaaaccagggaaagcccctaagctcctgatctataatgcaaaaaccttagcaaaaggggtcccatcaaggttcagtggcagtggatctgggacagatttcactctcaccatcagcagtctgcaacctgaagattttgcaacttactactgtcaacatcattatgagagtcctctgacgttcggcggagggaccaaggtggagatcaaacgtacg

SEQ ID NO: 52 - protein, 107 aa, synthetic construct
  REGION 1..107: note = an artificially synthesized sequence
DIQMTQSPSSLSASVGDRVTITCRTSENIYSFLAWYQQKPGKAPKLLIYNAKTLAKGVPSRFSGSGSGTDFTLTISSLQPEDFATYYCQHHYESPLTFGGGTKVEIK

SEQ ID NO: 53 - DNA, 1422 bp, synthetic construct
  misc_feature 1..1422: note = an artificially synthesized sequence
  source 1..1422: mol_type = other DNA
gaattccaccatggactggacctggagggtcttctgcttgctggctgtagctccaggtgctcactcccaggtgcagctggtgcagtctggggctgaggtgaagaagcctggggcctcagtgaaggtttcctgcaaggcatctggatacaccttcaccggctacatcatgaactgggtgcgacaggcccctggacaagggcttgagtggatgggacttattaatccttacaatggtggtactagctacaaccagaagttcaagggcagagtcacgattaccgcggacgaatccacgagcacagcctacatggagctgagcagcctgagatctgaggacacggccgtgtattactgtgcgagagatggttacgacgacggaccctatactatggactactggggccagggcaccctcgtcacagtctcctcagctagcaccaagggcccatcggtcttccccctggcgccctcctccaagagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatcttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgataagcggccgc

SEQ ID NO: 54 - protein, 447 aa, synthetic construct
  REGION 1..447: note = an artificially synthesized sequence
QVQLVQSGAEVKKPGASVKVSCKASGYTFTGYIMNWVRQAPGQGLEWMGLINPYNGGTSYNQKFKGRVTITADESTSTAYMELSSLRSEDTAVYYCARDGYDDGPYTMDYWGQGTLVTVSSASTKGPSVFPLAPSSKSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 55 - DNA, 719 bp, synthetic construct
  misc_feature 1..719: note = an artificially synthesized sequence
  source 1..719: mol_type = other DNA
ccaccatggacatgagggtccccgctcagctcctggggctcctgctactctggctccgaggtgccagatgtgacatccagatgacccagtctccatcctccctgtctgcatctgtaggagacagagtcaccatcacttgccgaacaagtgagaatatttacagttttttagcatggtatcagcagaaaccagggaaagcccctaagctcctgatctataatgcaaaaaccttagcaaaaggggtcccatcaaggttcagtggcagtggatctgggacagatttcactctcaccatcagcagtctgcaacctgaagattttgcaacttactactgtcaacatcattatgagagtcctctgacgttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgataa

SEQ ID NO: 56 - protein, 214 aa, synthetic construct
  REGION 1..214: note = an artificially synthesized sequence
DIQMTQSPSSLSASVGDRVTITCRTSENIYSFLAWYQQKPGKAPKLLIYNAKTLAKGVPSRFSGSGSGTDFTLTISSLQPEDFATYYCQHHYESPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - DNA, 327 bp, Homo sapiens
  source 1..327: mol_type = unassigned DNA
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgataa

SEQ ID NO: 58 - protein, 107 aa, Homo sapiens [sequence omitted]

SEQ ID NO: 59 - DNA, 990 bp, Homo sapiens
  source 1..990: mol_type = unassigned DNA
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 60 - protein, 330 aa, Homo sapiens [sequence omitted]

SEQ ID NO: 61 - DNA, 984 bp, Homo sapiens
  source 1..984: mol_type = unassigned DNA
gctagcaccaagggcccatcggtcttccccctggcgccctcctccaagagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatcttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgataa

SEQ ID NO: 62 - protein, 326 aa, Homo sapiens
ASTKGPSVFPLAPSSKSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 63 - DNA, 995 bp, Homo sapiens
  source 1..995: mol_type = unassigned DNA
gctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggttaatgataagcggccgc

SEQ ID NO: 64 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 65 - DNA, 2208 bp, Macaca fascicularis
  source 1..2208: mol_type = unassigned DNA
atgatgtggacctgggcactgtggatgttccctttactctgcaaattcggcctggcagctctgccagctaagcctgagaacatttcctgtgtctactactataggaaaaatttaacctgcacttggagtccaggaaaggaaactagttatacccagtacacagctaagagaacttacgcttttggaaaaaaacatgataattgtacaaccagtagttctacaagtgaaaatcgtgcttcgtgctcttttttccttccaagaataacgatcccagataattataccattgaggtggaagctgaaaatggagatggtgtaattaaatctgatatgacatgttggagattagaggacatagcgaaaactgaaccacctgagattttcagtgtgaaaccagttttgggcatcaaacgaatgattcggattgaatggataaagcctgagttggcacctgtttcatctgatttaaaatatgcacttcgattcaggacagtcaatagtaccagctggatggaagtcaacttcgctaagaaccgtaaagatacaaaccaaacctacaaccttatggggctgcaggcttttacagagtatgtcgtagctctgcgatgtgcggtcaaggagtcaaagttctggagtgactggagccaagaaaaaatgggaatgactgaggaagaagctccatgtggcctggaactgtggagagtcctgaaaccaactgaggtggatggaagaaggccagtgcggttgttatggaagaaggcaagaggagccccagtcctagagaaaacacttggctacaacatatggtactttccagaaaacaacactaacctcacagagacagtgaacaccactaaccagcagcttgaactgcatctgggaggcgagagctattgggtgtctatgatttcttataattctcttgggaagtctccagtgaccaccctgaggattccagccattcaggaaaagtcatttcggtgcattgaggtcatgcaggcctgccttgctgaggaccagctagtggtgaagtggcaaagctctgctctagacgtgaacacttggatgattgaatggtttccggacatggactcagagcaccccactctttcctgggaatctgtgtctcaggccacgaactggacaatccagcaagataaattaaaacctttctggtgctataacatctctgtgtatccaatgttgcacgacaaagttggcgagccatattccatccaggcttatgccaaagaaggcattccatcaaaaggtcctgagaccaaggtggagaacattggcgtgaagacggtcacgatcacatggaaagagattcccaagagtgagagaaagggtatcatctgcaactacaccatcttttaccaagctgaaggtggaaaaggattctccaagacagtcaactccagcatcttgcagtatggcctggagtccctgaaacgaaagacctcttacactgttcgggtcatggccagcaccagtgctgggggaatcaacgggaccagcataaatttcaagacattgtcattcagtgtttttgagattatccttataacttctctgattggtggaggccttcttattctcattatcctgacggtggcatatggtctcaaaaaacccaacaaattgactcacctgtgttggcccagtgttcccaaccctgctgaaagtagtatagccacatggcgtggagatgatttcaaggataagctaaacctgaaggagtctgatgactctgtgaacacagaagacaggatcttaaaaccatgttccacccccagtgacaagttggttattgacaagtcggtggtgaactttgggaatgttctgcaagaaatgttcacagatgaagccagaacgggtcaggaaaacaatttaggaggggaaaagaatgagtatgtgacccaccccttcagggctgactgtcccctggggaaaagttttgaggagctcccagtttcacctgagattcctcccagaaaatcccaatacctacgttcgaggatgccagaagggacctgcctagaagccgaagagcagcttctcgtttctggtcaaagtctagaaagtctagcaccagaccatgtgcgggaggcagcggccccaaatccgtatttgaaaaattcagtgacaaccagggaatttcttgtgtctcaaaaacttccagagcacaccaaaggagaagtctaa

SEQ ID NO: 66 - protein, 735 aa, Macaca fascicularis
MMWTWALWMFPLLCKFGLAALPAKPENISCVYYYRKNLTCTWSPGKETSYTQYTAKRTYAFGKKHDNCTTSSSTSENRASCSFFLPRITIPDNYTIEVEAENGDGVIKSDMTCWRLEDIAKTEPPEIFSVKPVLGIKRMIRIEWIKPELAPVSSDLKYALRFRTVNSTSWMEVNFAKNRKDTNQTYNLMGLQAFTEYVVALRCAVKESKFWSDWSQEKMGMTEEEAPCGLELWRVLKPTEVDGRRPVRLLWKKARGAPVLEKTLGYNIWYFPENNTNLTETVNTTNQQLELHLGGESYWVSMISYNSLGKSPVTTLRIPAIQEKSFRCIEVMQACLAEDQLVVKWQSSALDVNTWMIEWFPDMDSEHPTLSWESVSQATNWTIQQDKLKPFWCYNISVYPMLHDKVGEPYSIQAYAKEGIPSKGPETKVENIGVKTVTITWKEIPKSERKGIICNYTIFYQAEGGKGFSKTVNSSILQYGLESLKRKTSYTVRVMASTSAGGINGTSINFKTLSFSVFEIILITSLIGGGLLILIILTVAYGLKKPNKLTHLCWPSVPNPAESSIATWRGDDFKDKLNLKESDDSVNTEDRILKPCSTPSDKLVIDKSVVNFGNVLQEMFTDEARTGQENNLGGEKNEYVTHPFRADCPLGKSFEELPVSPEIPPRKSQYLRSRMPEGTCLEAEEQLLVSGQSLESLAPDHVREAAAPNPYLKNSVTTREFLVSQKLPEHTKGEV

SEQ ID NO: 67 - DNA, 495 bp, Macaca fascicularis
  source 1..495: mol_type = unassigned DNA
atggcctctcactcagcaggccccgcgacgtccgtgctgtttctgctctgctgcctgggaggctggctgacctcccacacgttgcccgtccatttcctacaaccaagtgatatacagaaaatagtcgaggaattacagtccctctcgaagatgcttttgaaagatgtgaaggaagacaagggggtgctcgtgtcccagaattacacgctgccgtgtctcacccctgacgcccagccgccaaacatcatccacagcccagccatccgggcatatctcaagacaatcagacagttagacaacaaatctgttattgatgagatcatagagcacctcgacaaactcatatttcaagatgcaccagaaacaaacatttctgtgccaacagacacccatgaatgtaaacgcttcatcctgactatttctcaacagttttcagagtgcatggaccttgcattaaaatcgttgacttctggagcccagcaggccaccacttaa

SEQ ID NO: 68 - protein, 164 aa, Macaca fascicularis
MASHSAGPATSVLFLLCCLGGWLTSHTLPVHFLQPSDIQKIVEELQSLSKMLLKDVKEDKGVLVSQNYTLPCLTPDAQPPNIIHSPAIRAYLKTIRQLDNKSVIDEIIEHLDKLIFQDAPETNISVPTDTHECKRFILTISQQFSECMDLALKSLTSGAQQATT

SEQ ID NO: 69 - DNA, 2934 bp, Macaca fascicularis
  source 1..2934: mol_type = unassigned DNA
atggctctatttgtagtctttcagacaacattcttcttaacattgctgtccttgaggacttaccagagtgaagtcttggctgaacgtttaccattgactcctgtgtcacttaaagtttccaccaattctatacatcagagtttgcatttacaatggactgtccacaaccttccttatcatcaggaattgaaaatggtatttcagatccagatcagtaggattgaaacatccaatgtcgtctgggtggggaattacagcaccactgtgaagtggaaccaggttctgcattggagctgggaatcggaactccctttggaatgtgccacacactttgtaagaatcaagagtgtgatagacgatgccagtttccctgagccaaatttctggagcaactggagttcctgggaggaagtcagtgtacaagattatcttggacggggcactttgttcgttttccctaaagataagctggtggaagaaggctccaatgttaccatttgttatgtttctaggaacattcaaaataatgtatcctgttatttggaagggaaacagattcacggagaacaacttgatccacatgtaactgcattcaacttgaatagtgtgcctttcattaggaatagagggacaaatatctattgtgaggcgagtcaaggaaatgtcagtaaaggcatagaaggcatcgttctctttgtctcaaaagtacttgaggagcccaaggacttttcttgtgaatcccaggacttcaacactttgcactgtacttgggatcctgggacggacactgccttggggtggtctaaacaaccttcccaaagctacactttatttgaatcattttctggggaaaagaaactttgtacgcacaaaaactggtgtaattggcaaataactcaagactcacaagaaatgtataacttcacactcatagctgaaaattacttaaggaagagaagtgtcaatatcctttttaacctgactcatcgagtttatttaatgaatccttttagtgtcaactttgaaaatgtaaatgccacaaatgccatcatgacctggaaggtgcactccatgaggaataatttcacatatttgtgtcagattgaactccatggtgaaggaaaaatgatgcaatacgatgtttctatcaacgtgaacggtgagtacttcttaagtgaactggaacctgccacagaatatatggcccgagtacgctgtgctgatgccagccacttctggaaatggactgaatggagtggtcagaacttcaccacacttgaagctgctccgtcagaggcccctgatgtctggagaagtgtgaactcagagccaggaaatcatactgtgaccttattctggaagccattatcaaaactgcatgccaatggaaagatcctgttctataatgtagttgtagaaaacctagacaaaccgtccaggtcagagctccgttccattccggcaccagccaacagcacaaaactaatcctcgacaggtgttcctaccaaatctgcgtcacagctaacaacagtgtgggcgcttctcctgcttctataatagtcatctctgcggaccctgaaaacaaagaggttgaggaagaaagaattgcaggcacagagggtggattctctctgtcttggaaaccccagcctggagatgttataggctatgttgtggactggtgtgaccatccccaggatgtgctccagtggaagaatgtaggtcccaataccacaagcacagtcattagcacagatgcttttaggccaggagttcgatacgacttcagaatctatgggttatctacaaaaaggattgcttgtttattagagaaaaaaacaggatactctcaggaactggctccttcagacaaccctcacgtgctggtagatatgttgacatcccactccttcactctgagttggaaagattactctactgaatctcaacctggttttatacaagggtaccatgtctatctgaaatccaaggcgaggcagtgccacccacgatttcaaaaggcagttctttcagatggttcagaatgttgcaaatacaaaattgacaacccagaagaaaaggcattgattgtggacaacctaaagccagaatccttctatgagtttttcgttactccattcactagtgctggcgagggccccaatgctacgttcacgaaggtcacgactccggatgaacactcctccatgttgattcgtatcctactgcccatggttttctgcgtcttgctcatcatgatcgtgtgctacttgaaaagtcagtggatcaaggagacgtgttatcctgacatccctgacccttacaagagcagcatcctgtcgttaataaaattcaaggagaaccctcacctaacaataatgaatgtcagtgactgtatcccagatgctattgaagttgtcagcaagccagaagggacaaagatacagctcctaggcactaggaagtcactcacagaaactgagttaactaagcctaactacctttatctccttccaacagaaaagaatcactctggccctggcccctgcatctgttttgagaactttacctacaaccaggcagcttctgacgctggctcttgtggccatgttccagtaccccccaaagccccaccaagtatgctaggactaatgacctcacctgaaaatgtactaaaggcgctagaaaaaaactacatgaactccctgggagaagtcccagctggagaaacaagtttgaattatgtgtcccagttggcttcacccatgtctggagacaaggacagtctcccaacaaacccagtggagccaccacactgttcagagtataaaatgcaaatggcagtccccctgcgtcttgccctgcctcccccgaccgagaatagcagcctttcctcaattacccttttagatccaggtgaacactaccgctaa

SEQ ID NO: 70 - protein, 977 aa, Macaca fascicularis
MALFVVFQTTFFLTLLSLRTYQSEVLAERLPLTPVSLKVSTNSIHQSLHLQWTVHNLPYHQELKMVFQIQISRIETSNVVWVGNYSTTVKWNQVLHWSWESELPLECATHFVRIKSVIDDASFPEPNFWSNWSSWEEVSVQDYLGRGTLFVFPKDKLVEEGSNVTICYVSRNIQNNVSCYLEGKQIHGEQLDPHVTAFNLNSVPFIRNRGTNIYCEASQGNVSKGIEGIVLFVSKVLEEPKDFSCESQDFNTLHCTWDPGTDTALGWSKQPSQSYTLFESFSGEKKLCTHKNWCNWQITQDSQEMYNFTLIAENYLRKRSVNILFNLTHRVYLMNPFSVNFENVNATNAIMTWKVHSMRNNFTYLCQIELHGEGKMMQYDVSINVNGEYFLSELEPATEYMARVRCADASHFWKWTEWSGQNFTTLEAAPSEAPDVWRSVNSEPGNHTVTLFWKPLSKLHANGKILFYNVVVENLDKPSRSELRSIPAPANSTKLILDRCSYQICVTANNSVGASPASIIVISADPENKEVEEERIAGTEGGFSLSWKPQPGDVIGYVVDWCDHPQDVLQWKNVGPNTTSTVISTDAFRPGVRYDFRIYGLSTKRIACLLEKKTGYSQELAPSDNPHVLVDMLTSHSFTLSWKDYSTESQPGFIQGYHVYLKSKARQCHPRFQKAVLSDGSECCKYKIDNPEEKALIVDNLKPESFYEFFVTPFTSAGEGPNATFTKVTTPDEHSSMLIRILLPMVFCVLLIMIVCYLKSQWIKETCYPDIPDPYKSSILSLIKFKENPHLTIMNVSDCIPDAIEVVSKPEGTKIQLLGTRKSLTETELTKPNYLYLLPTEKNHSGPGPCICFENFTYNQAASDAGSCGHVPVPPKAPPSMLGLMTSPENVLKALEKNYMNSLGEVPAGETSLNYVSQLASPMSGDKDSLPTNPVEPPHCSEYKMQMAVPLRLALPPPTENSSLSSITLLDPGEHYR

SEQ ID NO: 71 - DNA, 1665 bp, Macaca fascicularis
  source 1..1665: mol_type = unassigned DNA
atgatgtggacctgggcactgtggatgttccctttactctgcaaattcggcctggcagctctgccagctaagcctgagaacatttcctgtgtctactactataggaaaaatttaacctgcacttggagtccaggaaaggaaactagttatacccagtacacagctaagagaacttacgcttttggaaaaaaacatgataattgtacaaccagtagttctacaagtgaaaatcgtgcttcgtgctcttttttccttccaagaataacgatcccagataattataccattgaggtggaagctgaaaatggagatggtgtaattaaatctgatatgacatgttggagattagaggacatagcgaaaactgaaccacctgagattttcagtgtgaaaccagttttgggcatcaaacgaatgattcggattgaatggataaagcctgagttggcacctgtttcatctgatttaaaatatgcacttcgattcaggacagtcaatagtaccagctggatggaagtcaacttcgctaagaaccgtaaagatacaaaccaaacctacaaccttatggggctgcaggcttttacagagtatgtcgtagctctgcgatgtgcggtcaaggagtcaaagttctggagtgactggagccaagaaaaaatgggaatgactgaggaagaagctccatgtggcctggaactgtggagagtcctgaaaccaactgaggtggatggaagaaggccagtgcggttgttatggaagaaggcaagaggagccccagtcctagagaaaacacttggctacaacatatggtactttccagaaaacaacactaacctcacagagacagtgaacaccactaaccagcagcttgaactgcatctgggaggcgagagctattgggtgtctatgatttcttataattctcttgggaagtctccagtgaccaccctgaggattccagccattcaggaaaagtcatttcggtgcattgaggtcatgcaggcctgccttgctgaggaccagctagtggtgaagtggcaaagctctgctctagacgtgaacacttggatgattgaatggtttccggacatggactcagagcaccccactctttcctgggaatctgtgtctcaggccacgaactggacaatccagcaagataaattaaaacctttctggtgctataacatctctgtgtatccaatgttgcacgacaaagttggcgagccatattccatccaggcttatgccaaagaaggcattccatcaaaaggtcctgagaccaaggtggagaacattggcgtgaagacggtcacgatcacatggaaagagattcccaagagtgagagaaagggtatcatctgcaactacaccatcttttaccaagctgaaggtggaaaaggattctccaagacagtcaactccagcatcttgcagtatggcctggagtccctgaaacgaaagacctcttacactgttcgggtcatggccagcaccagtgctgggggaatcaacgggaccagcataaatttcaagacattgtcattcagtgtttttgagattatccttataacttctctgattggtggaggccttcttattctcattatcctgacggtggcatatggtctcaaaaaacccaacaaattgactcacctgtgttaatga

SEQ ID NO: 72 - protein, 553 aa, Macaca fascicularis
MMWTWALWMFPLLCKFGLAALPAKPENISCVYYYRKNLTCTWSPGKETSYTQYTAKRTYAFGKKHDNCTTSSSTSENRASCSFFLPRITIPDNYTIEVEAENGDGVIKSDMTCWRLEDIAKTEPPEIFSVKPVLGIKRMIRIEWIKPELAPVSSDLKYALRFRTVNSTSWMEVNFAKNRKDTNQTYNLMGLQAFTEYVVALRCAVKESKFWSDWSQEKMGMTEEEAPCGLELWRVLKPTEVDGRRPVRLLWKKARGAPVLEKTLGYNIWYFPENNTNLTETVNTTNQQLELHLGGESYWVSMISYNSLGKSPVTTLRIPAIQEKSFRCIEVMQACLAEDQLVVKWQSSALDVNTWMIEWFPDMDSEHPTLSWESVSQATNWTIQQDKLKPFWCYNISVYPMLHDKVGEPYSIQAYAKEGIPSKGPETKVENIGVKTVTITWKEIPKSERKGIICNYTIFYQAEGGKGFSKTVNSSILQYGLESLKRKTSYTVRVMASTSAGGINGTSINFKTLSFSVFEIILITSLIGGGLLILIILTVAYGLKKPNKLTHLC

SEQ ID NO: 73 - DNA, 1665 bp, Homo sapiens
  source 1..1665: mol_type = unassigned DNA
atgatgtggacctgggcactgtggatgctcccctcactctgcaaattcagcctggcagctctgccagctaagcctgagaacatttcctgtgtctactactataggaaaaatttaacctgcacttggagtccaggaaaggaaaccagttatacccagtacacagttaagagaacttacgcttttggagaaaaacatgataattgtacaaccaatagttctacaagtgaaaatcgtgcttcgtgctcttttttccttccaagaataacgatcccagataattataccattgaggtggaagctgaaaatggagatggtgtaattaaatctcatatgacatactggagattagagaacatagcgaaaactgaaccacctaagattttccgtgtgaaaccagttttgggcatcaaacgaatgattcaaattgaatggataaagcctgagttggcgcctgtttcatctgatttaaaatacacacttcgattcaggacagtcaacagtaccagctggatggaagtcaacttcgctaagaaccgtaaggataaaaaccaaacgtacaacctcacggggctgcagccttttacagaatatgtcatagctctgcgatgtgcggtcaaggagtcaaagttctggagtgactggagccaagaaaaaatgggaatgactgaggaagaagctccatgtggcctggaactgtggagagtcctgaaaccagctgaggcggatggaagaaggccagtgcggttgttatggaagaaggcaagaggagccccagtcctagagaaaacacttggctacaacatatggtactatccagaaagcaacactaacctcacagaaacaatgaacactactaaccagcagcttgaactgcatctgggaggcgagagcttttgggtgtctatgatttcttataattctcttgggaagtctccagtggccaccctgaggattccagctattcaagaaaaatcgtttcagtgcattgaggtcatgcaggcctgcgttgctgaggaccagctagtggtgaagtggcaaagctctgctctagacgtgaacacttggatgattgaatggtttccggatgtggactcagagcccaccaccctttcctgggaatctgtgtctcaggccacgaactggacgatccagcaagataaattaaaacctttctggtgctataacatctctgtgtatccaatgttgcatgacaaagttggcgagccatattccatccaggcttatgccaaagaaggcgttccatcagaaggtcctgagaccaaggtggagaacattggcgtgaagacggtcacgatcacatggaaagagattcccaagagtgagagaaagggtatcatctgcaactacaccatcttttaccaagctgaaggtggaaaaggattctccaagacagtcaattccagcatcttgcagtacggcctggagtccctgaaacgaaagacctcttacattgttcaggtcatggccagcaccagtgctgggggaaccaacgggaccagcataaatttcaagacattgtcattcagtgtctttgagattatcctcataacttctctgattggtggaggccttcttattctcattatcctgacagtggcatatggtctcaaaaaacccaacaaattgactcatctgtgttaatga

SEQ ID NO: 74 - protein, 553 aa, Homo sapiens
MMWTWALWMLPSLCKFSLAALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSSALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMASTSAGGTNGTSINFKTLSFSVFEIILITSLIGGGLLILIILTVAYGLKKPNKLTHLC

SEQ ID NO: 75 - DNA, 2199 bp, Homo sapiens
  source 1..2199: mol_type = unassigned DNA
atgatgtggacctgggcactgtggatgctcccctcactctgcaaattcagcctggcagctctgccagctaagcctgagaacatttcctgtgtctactactataggaaaaatttaacctgcacttggagtccaggaaaggaaaccagttatacccagtacacagttaagagaacttacgcttttggagaaaaacatgataattgtacaaccaatagttctacaagtgaaaatcgtgcttcgtgctcttttttccttccaagaataacgatcccagataattataccattgaggtggaagctgaaaatggagatggtgtaattaaatctcatatgacatactggagattagagaacatagcgaaaactgaaccacctaagattttccgtgtgaaaccagttttgggcatcaaacgaatgattcaaattgaatggataaagcctgagttggcgcctgtttcatctgatttaaaatacacacttcgattcaggacagtcaacagtaccagctggatggaagtcaacttcgctaagaaccgtaaggataaaaaccaaacgtacaacctcacggggctgcagccttttacagaatatgtcatagctctgcgatgtgcggtcaaggagtcaaagttctggagtgactggagccaagaaaaaatgggaatgactgaggaagaagctccatgtggcctggaactgtggagagtcctgaaaccagctgaggcggatggaagaaggccagtgcggttgttatggaagaaggcaagaggagccccagtcctagagaaaacacttggctacaacatatggtactatccagaaagcaacactaacctcacagaaacaatgaacactactaaccagcagcttgaactgcatctgggaggcgagagcttttgggtgtctatgatttcttataattctcttgggaagtctccagtggccaccctgaggattccagctattcaagaaaaatcgtttcagtgcattgaggtcatgcaggcctgcgttgctgaggaccagctagtggtgaagtggcaaagctctgctctagacgtgaacacttggatgattgaatggtttccggatgtggactcagagcccaccaccctttcctgggaatctgtgtctcaggccacgaactggacgatccagcaagataaattaaaacctttctggtgctataacatctctgtgtatccaatgttgcatgacaaagttggcgagccatattccatccaggcttatgccaaagaaggcgttccatcagaaggtcctgagaccaaggtggagaacattggcgtgaagacggtcacgatcacatggaaagagattcccaagagtgagagaaagggtatcatctgcaactacaccatcttttaccaagctgaaggtggaaaaggattctccaagacagtcaattccagcatcttgcagtacggcctggagtccctgaaacgaaagacctcttacattgttcaggtcatggccagcaccagtgctgggggaaccaacgggaccagcataaatttcaagacattgtcattcagtgtctttgagattatcctcataacttctctgattggtggaggccttcttattctcattatcctgacagtggcatatggtctcaaaaaacccaacaaattgactcatctgtgttggcccaccgttcccaaccctgctgaaagtagtatagccacatggcatggagatgatttcaaggataagctaaacctgaaggagtctgatgactctgtgaacacagaagacaggatcttaaaaccatgttccacccccagtgacaagttggtgattgacaagttggtggtgaactttgggaatgttctgcaagaaattttcacagatgaagccagaacgggtcaggaaaacaatttaggaggggaaaagaatgggtatgtgacctgccccttcaggcctgattgtcccctggggaaaagttttgaggagctcccagtttcacctgagattccgcccagaaaatcccaatacctacgttcgaggatgccagaggggacccgcccagaagccaaagagcagcttctcttttctggtcaaagtttagtaccagatcatctgtgtgaggaaggagccccaaatccatatttgaaaaattcagtgacagccagggaatttcttgtgtctgaaaaacttccagagcacaccaagggagaagtctaa

SEQ ID NO: 76 - protein, 732 aa, Homo sapiens
MMWTWALWMLPSLCKFSLAALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSSALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMASTSAGGTNGTSINFKTLSFSVFEIILITSLIGGGLLILIILTVAYGLKKPNKLTHLCWPTVPNPAESSIATWHGDDFKDKLNLKESDDSVNTEDRILKPCSTPSDKLVIDKLVVNFGNVLQEIFTDEARTGQENNLGGEKNGYVTCPFRPDCPLGKSFEELPVSPEIPPRKSQYLRSRMPEGTRPEAKEQLLFSGQSLVPDHLCEEGAPNPYLKNSVTAREFLVSEKLPEHTKGEV

SEQ ID NO: 77 - DNA, 1542 bp, Homo sapiens
  source 1..1542: mol_type = unassigned DNA
atgatgtggacctgggcactgtggatgctcccttcactctgcaaattcagcctggcagctctgccagctaagcctgagaacatttcctgtgtctactactataggaaaaatttaacctgcacttggagtccaggaaaggaaaccagttatacccagtacacagttaagagaacttacgcttttggagaaaaacatgataattgtacaaccaatagttctacaagtgaaaatcgtgcttcgtgctcttttttccttccaagaataacgatcccagataattataccattgaggtggaagctgaaaatggagatggtgtaattaaatctcatatgacatactggagattagagaacatagcgaaaactgaaccacctaagattttccgtgtgaaaccagttttgggcatcaaacgaatgattcaaattgaatggataaagcctgagttggcgcctgtttcatctgatttaaaatacacacttcgattcaggacagtcaacagtaccagctggatggaagtcaacttcgctaagaaccgtaaggataaaaaccaaacgtacaacctcacggggctgcagccttttacagaatatgtcatagctctgcgatgtgcggtcaaggagtcaaagttctggagtgactggagccaagaaaaaatgggaatgactgaggaagaagctccatgtggcctggaactgtggagagtcctgaaaccagctgaggcggatggaagaaggccagtgcggttgttatggaagaaggcaagaggagccccagtcctagagaaaacacttggctacaacatatggtactatccagaaagcaacactaacctcacagaaacaatgaacactactaaccagcagcttgaactgcatctgggaggcgagagcttttgggtgtctatgatttcttataattctcttgggaagtctccagtggccaccctgaggattccagctattcaagaaaaatcgtttcagtgcattgaggtcatgcaggcctgcgttgctgaggaccagctagtggtgaagtggcaaagctctgctctagacgtgaacacttggatgattgaatggtttccggatgtggactcagagcccaccaccctttcctgggaatctgtgtctcaggccacgaactggacgatccagcaagataaattaaaacctttctggtgctataacatctctgtgtatccaatgttgcatgacaaagttggcgagccatattccatccaggcttatgccaaagaaggcgttccatcagaaggtcctgagaccaaggtggagaacattggcgtgaagacggtcacgatcacatggaaagagattcccaagagtgagagaaagggtatcatctgcaactacaccatcttttaccaagctgaaggtggaaaaggattctccaagacagtcaattccagcatcttgcagtacggcctggagtccctgaaacgaaagacctcttacattgttcaggtcatggccagcaccagtgctgggggaaccaacgggaccagcataaatttcaagacattgtca

SEQ ID NO: 78 - protein, 514 aa, Homo sapiens
MMWTWALWMLPSLCKFSLAALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSSALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMASTSAGGTNGTSINFKTLS

SEQ ID NO: 79 - protein, 662 aa, Homo sapiens
MKLSPQPSCVNLGMMWTWALWMLPSLCKFSLAALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSSALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMASTSAGGTNGTSINFKTLSFSVFEIILITSLIGGGLLILIILTVAYGLKKPNKLTHLCWPTVPNPAESSIATWHGDDFKDKLNLKESDDSVNTEDRILKPCSTPSDKLVIDKLVVNFGNVLQEIFTDEARTGQENNLGGEKNGTRILSSCPTSI

SEQ ID NO: 80 - protein, 732 aa, Homo sapiens; identical to SEQ ID NO: 76
  SIGNAL 1..20
  TRANSMEM 520..543

SEQ ID NO: 81 - protein, 716 aa, Mus musculus
MWTLALWAFSFLCKFSLAVLPTKPENISCVFYFDRNLTCTWRPEKETNDTSYIVTLTYSYGKSNYSDNATEASYSFPRSCAMPPDICSVEVQAQNGDGKVKSDITYWHLISIAKTEPPIILSVNPICNRMFQIQWKPREKTRGFPLVCMLRFRTVNSSRWTEVNFENCKQVCNLTGLQAFTEYVLALRFRFNDSRYWSKWSKEETRVTMEEVPHVLDLWRILEPADMNGDRKVRLLWKKARGAPVLEKTFGYHIQYFAENSTNLTEINNITTQQYELLLMSQAHSVSVTSFNSLGKSQEAILRIPDVHEKTFQYIKSMKAYIAEPLLVVNWQSSIPAVDTWIVEWLPEAAMSKFPALSWESVSQVTNWTIEQDKLKPFTCYNISVYPVLGHRVGEPYSIQAYAKEGTPLKGPETRVENIGLRTATITWKEIPKSARNGFINNYTVFYQAEGGKELSKTVNSHALQCDLESLTRRTSYTVWVMASTRAGGTNGVRINFKTLSISVFEIVLLTSLVGGGLLLLSIKTVTFGLRKPNRLTPLCCPDVPNPAESSLATWLGDGFKKSNMKETGNSGDTEDVVLKPCPVPADLIDKLVVNFENFLEVVLTEEAGKGQASILGGEANEYVTSPSRPDGPPGKSFKEPSVLTEVASEDSHSTCSRMADEAYSELARQPSSSCQSPGLSPPREDQAQNPYLKNSVTTREFLVHENIPEHSKGEV

SEQ ID NO: 82 - protein, 4 aa, synthetic construct
  REGION 1..4: note = an artificially synthesized peptide sequence
GGGS

SEQ ID NO: 83 - protein, 4 aa, synthetic construct
  REGION 1..4: note = an artificially synthesized peptide sequence
SGGG

SEQ ID NO: 84 - protein, 5 aa, synthetic construct
  REGION 1..5: note = an artificially synthesized peptide sequence
GGGGS

SEQ ID NO: 85 - protein, 5 aa, synthetic construct
  REGION 1..5: note = an artificially synthesized peptide sequence
SGGGG

SEQ ID NO: 86 - protein, 6 aa, synthetic construct
  REGION 1..6: note = an artificially synthesized peptide sequence
GGGGGS

SEQ ID NO: 87 - protein, 6 aa, synthetic construct
  REGION 1..6: note = an artificially synthesized peptide sequence
SGGGGG

SEQ ID NO: 88 - protein, 7 aa, synthetic construct
  REGION 1..7: note = an artificially synthesized peptide sequence
GGGGGGS

SEQ ID NO: 89 - protein, 7 aa, synthetic construct
  REGION 1..7: note = an artificially synthesized peptide sequence
SGGGGGG

SEQ ID NO: 90 - DNA, 40 bp, synthetic construct
  misc_feature 1..40: note = an artificially synthesized primer sequence
  source 1..40: mol_type = other DNA
taatagcggccgctcattatttaccaggagagtgggagag

SEQ ID NO: 91 - DNA, 40 bp, synthetic construct
  misc_feature 1..40: note = an artificially synthesized primer sequence
  source 1..40: mol_type = other DNA
taatagcggccgctcattaacactcattcctgttgaagct

SEQ ID NO: 92 - DNA, 33 bp, synthetic construct
  misc_feature 1..33: note = an artificially synthesized primer sequence
  source 1..33: mol_type = other DNA
gacgaattccaccatgggatggagctggatctt

SEQ ID NO: 93 - DNA, 33 bp, synthetic construct
  misc_feature 1..33: note = an artificially synthesized primer sequence
  source 1..33: mol_type = other DNA
gacgaattccaccatgagtgtgcccactcaggt

SEQ ID NO: 94 - DNA, 33 bp, synthetic construct
  misc_feature 1..33: note = an artificially synthesized primer sequence
  source 1..33: mol_type = other DNA
gacgaattccaccatggaatgtaactggatact

SEQ ID NO: 95 - DNA, 33 bp, synthetic construct
  misc_feature 1..33: note = an artificially synthesized primer sequence
  source 1..33: mol_type = other DNA
gacgaattccaccatggattttctggtgcagat

SEQ ID NO: 96 - protein, 30 aa, Homo sapiens
QVQLVQSGAEVKKPGASVKVSCKASGYTFT

SEQ ID NO: 97 - protein, 14 aa, Homo sapiens
WVRQAPGQGLEWMG

SEQ ID NO: 98 - protein, 32 aa, Homo sapiens
RVTITADESTSTAYMELSSLRSEDTAVYYCAR

SEQ ID NO: 99 - protein, 11 aa, Homo sapiens
WGQGTLVTVSS

SEQ ID NO: 100 - protein, 23 aa, Homo sapiens
DIQMTQSPSSLSASVGDRVTITC

SEQ ID NO: 101 - protein, 15 aa, Homo sapiens
WYQQKPGKAPKLLIY

SEQ ID NO: 102 - protein, 32 aa, Homo sapiens
GVPSRFSGSGSGTDFTLTISSLQPEDFATYYC

SEQ ID NO: 103 - protein, 10 aa, Homo sapiens
FGGGTKVEIK

SEQ ID NO: 104 - protein, 119 aa, Mus musculus
DVQLRESGPGLVKPSQSLSLTCTVTGYSITSDYAWNWIRQFPGNKLEWMGYISYSGSTSYNPSLKSRVSITRDTSKNQFFLQLNSVTTEDTATYYCAPMITTDWFFDVWGAGTTVTVSS

SEQ ID NO: 105 - protein, 6 aa, Mus musculus
SDYAWN

SEQ ID NO: 106 - protein, 15 aa, Mus musculus
YISYSGSTSYNPSLK

SEQ ID NO: 107 - protein, 10 aa, Mus musculus
MITTDWFFDV

SEQ ID NO: 108 - protein, 107 aa, Mus musculus
DIVMTQSPATLSVTPGDRVSLSCRASHDISDFLHWYQQKSHESPRLLIKYASQSISGIPSRFSGSGSGSDFTLSINSVEPEDVGVYYCQNGHSFPWTFGGGTKLEIK

SEQ ID NO: 109 - protein, 11 aa, Mus musculus
RASHDISDFLH

SEQ ID NO: 110 - protein, 7 aa, Mus musculus
YASQSIS

SEQ ID NO: 111 - protein, 9 aa, Mus musculus
QNGHSFPWT

SEQ ID NO: 112 - protein, 121 aa, synthetic construct
  REGION 1..121: note = An artificially synthesized peptide sequence
QVQLVQSGAEVKKPGASVKVSCKASGYTFTGYIMNWVRQAPGQGLEWMGLINPYNGGTSYNQKFKGRVTITADKSTSTAYMELSSLRSEDTAVYYCARDGYDDGPYTMDYWGQGTLVTVSS

SEQ ID NO: 113 - protein, 17 aa, synthetic construct
  REGION 1..17: note = An artificially synthesized peptide sequence
EINPYNGGTSYNQKFKG

SEQ ID NO: 114 - protein, 17 aa, synthetic construct
  REGION 1..17: note = An artificially synthesized peptide sequence
LIDPYNGGTSYNQKFKG

SEQ ID NO: 115 - protein, 17 aa, synthetic construct
  REGION 1..17: note = An artificially synthesized peptide sequence
LINPYNGGTSYNDKFKG

SEQ ID NO: 116 - protein, 17 aa, synthetic construct
  REGION 1..17: note = An artificially synthesized peptide sequence
LINPYNGGTSYNQQFKG

SEQ ID NO: 117 - protein, 17 aa, synthetic construct
  REGION 1..17: note = An artificially synthesized peptide sequence
LINPYNGGTSYNQKFQG

SEQ ID NO: 118 - protein, 17 aa, synthetic construct
  REGION 1..17: note = An artificially synthesized peptide sequence
LINPYNGGTSYNQKFKD

SEQ ID NO: 119 - protein, 17 aa, synthetic construct
  REGION 1..17: note = An artificially synthesized peptide sequence
LINPYNGGTSYNQKFQD

SEQ ID NO: 120 - protein, 14 aa, synthetic construct
  REGION 1..14: note = An artificially synthesized peptide sequence
WVQQSPGQGLEWMG

SEQ ID NO: 121 - protein, 11 aa, synthetic construct
  REGION 1..11: note = An artificially synthesized peptide sequence
QTSENIYSFLA

SEQ ID NO: 122 - protein, 11 aa, synthetic construct
  REGION 1..11: note = An artificially synthesized peptide sequence
RTSEDIYSFLA

SEQ ID NO: 123 - protein, 7 aa, synthetic construct
  REGION 1..7: note = An artificially synthesized peptide sequence
DAKTLAK

SEQ ID NO: 124 - protein, 7 aa, synthetic construct
  REGION 1..7: note = An artificially synthesized peptide sequence
NAQTLAK

SEQ ID NO: 125 - protein, 7 aa, synthetic construct
  REGION 1..7: note = An artificially synthesized peptide sequence
NAKTEAK

SEQ ID NO: 126 - protein, 7 aa, synthetic construct
  REGION 1..7: note = An artificially synthesized peptide sequence
NAKTLAQ

SEQ ID NO: 127 - protein, 7 aa, synthetic construct
  REGION 1..7: note = An artificially synthesized peptide sequence
NAKTLAD

SEQ ID NO: 128 - protein, 324 aa, synthetic construct
  REGION 1..324: note = An artificially synthesized peptide sequence
ASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 129 - protein, 326 aa, synthetic construct
  REGION 1..326: note = An artificially synthesized peptide sequence
ASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 447 aa, synthetic construct
  REGION 1..447: note = An artificially synthesized peptide sequence
QVQLVQSGAEVKKPGASVKVSCKASGYTFTGYIMNWVRQAPGQGLEWMGLINPYNGGTSYNQKFKGRVTITADKSTSTAYMELSSLRSEDTAVYYCARDGYDDGPYTMDYWGQGTLVTVSSASTKGPSVFPLAPSSKSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 131 - protein, 32 aa, synthetic construct
  REGION 1..32: note = An artificially synthesized peptide sequence
RVTITADKSTSTAYMELSSLRSEDTAVYYCAR

SEQ ID NO: 132 - protein, 326 aa, synthetic construct
  REGION 1..326: note = An artificially synthesized peptide sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 133 - protein, 451 aa, synthetic construct
  REGION 1..451: note = An artificially synthesized peptide sequence
QVQLVQSGAEVKKPGASVKVSCKASGYTFTGYIMNWVRQAPGQGLEWMGLINPYNGGTSYNQKFKGRVTITADESTSTAYMELSSLRSEDTAVYYCARDGYDDGPYTMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 447 aa, synthetic construct
  REGION 1..447: note = An artificially synthesized peptide sequence
QVQLVQSGAEVKKPGASVKVSCKASGYTFTGYIMNWVRQAPGQGLEWMGLINPYNGGTSYNQKFKGRVTITADESTSTAYMELSSLRSEDTAVYYCARDGYDDGPYTMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 135 - protein, 447 aa, synthetic construct
  REGION 1..447: note = An artificially synthesized peptide sequence
QVQLVQSGAEVKKPGASVKVSCKASGYTFTGYIMNWVRQAPGQGLEWMGLINPYNGGTSYNQKFKGRVTITADESTSTAYMELSSLRSEDTAVYYCARDGYDDGPYTMDYWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 445 aa, synthetic construct
  REGION 1..445: note = An artificially synthesized peptide sequence
QVQLVQSGAEVKKPGASVKVSCKASGYTFTGYIMNWVRQAPGQGLEWMGLINPYNGGTSYNQKFKGRVTITADESTSTAYMELSSLRSEDTAVYYCARDGYDDGPYTMDYWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 137 - protein, 121 aa, synthetic construct
  REGION 1..121: note = An artificially synthesized peptide sequence
QVQLVQSGAEVKKPGASVKVSCKASGYTFTGYIMNWVQQSPGQGLEWMGLINPYNGGTSYNQKFKGRVTITADESTSTAYMELSSLRSEDTAVYYCARDGYDDGPYTMDYWGQGTLVTVSS

SEQ ID NO: 138 - protein, 121 aa, synthetic construct
  REGION 1..121: note = An artificially synthesized peptide sequence
QVQLVQSGAEVKKPGASVKVSCKASGYTFTGYIMNWVRQAPGQGLEWMGEINPYNGGTSYNQKFKGRVTITADESTSTAYMELSSLRSEDTAVYYCARDGYDDGPYTMDYWGQGTLVTVSS

SEQ ID NO: 139 - protein, 121 aa, synthetic construct
  REGION 1..121: note = An artificially synthesized peptide sequence
QVQLVQSGAEVKKPGASVKVSCKASGYTFTGYIMNWVRQAPGQGLEWMGLIDPYNGGTSYNQKFKGRVTITADESTSTAYMELSSLRSEDTAVYYCARDGYDDGPYTMDYWGQGTLVTVSS

SEQ ID NO: 140 - protein, 121 aa, synthetic construct
  REGION 1..121: note = An artificially synthesized peptide sequence
QVQLVQSGAEVKKPGASVKVSCKASGYTFTGYIMNWVRQAPGQGLEWMGLINPYNGGTSYNDKFKGRVTITADESTSTAYMELSSLRSEDTAVYYCARDGYDDGPYTMDYWGQGTLVTVSS

SEQ ID NO: 141 - protein, 121 aa, synthetic construct
  REGION 1..121: note = An artificially synthesized peptide sequence
QVQLVQSGAEVKKPGASVKVSCKASGYTFTGYIMNWVRQAPGQGLEWMGLINPYNGGTSYNQQFKGRVTITADESTSTAYMELSSLRSEDTAVYYCARDGYDDGPYTMDYWGQGTLVTVSS

SEQ ID NO: 142 - protein, 121 aa, synthetic construct
  REGION 1..121: note = An artificially synthesized peptide sequence
QVQLVQSGAEVKKPGASVKVSCKASGYTFTGYIMNWVRQAPGQGLEWMGLINPYNGGTSYNQKFQGRVTITADESTSTAYMELSSLRSEDTAVYYCARDGYDDGPYTMDYWGQGTLVTVSS

SEQ ID NO: 143 - protein, 121 aa, synthetic construct
  REGION 1..121: note = An artificially synthesized peptide sequence
QVQLVQSGAEVKKPGASVKVSCKASGYTFTGYIMNWVRQAPGQGLEWMGLINPYNGGTSYNQKFQDRVTITADESTSTAYMELSSLRSEDTAVYYCARDGYDDGPYTMDYWGQGTLVTVSS

SEQ ID NO: 144 - protein, 107 aa, synthetic construct
  REGION 1..107: note = An artificially synthesized peptide sequence
DIQMTQSPSSLSASVGDRVTITCQTSENIYSFLAWYQQKPGKAPKLLIYNAKTLAKGVPSRFSGSGSGTDFTLTISSLQPEDFATYYCQHHYESPLTFGGGTKVEIK

SEQ ID NO: 145 - protein, 107 aa, synthetic construct
  REGION 1..107: note = An artificially synthesized peptide sequence
DIQMTQSPSSLSASVGDRVTITCRTSEDIYSFLAWYQQKPGKAPKLLIYNAKTLAKGVPSRFSGSGSGTDFTLTISSLQPEDFATYYCQHHYESPLTFGGGTKVEIK

SEQ ID NO: 146 - protein, 107 aa, synthetic construct
  REGION 1..107: note = An artificially synthesized peptide sequence
DIQMTQSPSSLSASVGDRVTITCRTSENIYSFLAWYQQKPGKAPKLLIYDAKTLAKGVPSRFSGSGSGTDFTLTISSLQPEDFATYYCQHHYESPLTFGGGTKVEIK

SEQ ID NO: 147 - protein, 107 aa, synthetic construct
  REGION 1..107: note = An artificially synthesized peptide sequence
DIQMTQSPSSLSASVGDRVTITCRTSENIYSFLAWYQQKPGKAPKLLIYNAQTLAKGVPSRFSGSGSGTDFTLTISSLQPEDFATYYCQHHYESPLTFGGGTKVEIK

SEQ ID NO: 148 - protein, 107 aa, synthetic construct
  REGION 1..107: note = An artificially synthesized peptide sequence
DIQMTQSPSSLSASVGDRVTITCRTSENIYSFLAWYQQKPGKAPKLLIYNAKTEAKGVPSRFSGSGSGTDFTLTISSLQPEDFATYYCQHHYESPLTFGGGTKVEIK

SEQ ID NO: 149 - protein, 107 aa, synthetic construct
  REGION 1..107: note = An artificially synthesized peptide sequence
DIQMTQSPSSLSASVGDRVTITCRTSENIYSFLAWYQQKPGKAPKLLIYNAKTLAQGVPSRFSGSGSGTDFTLTISSLQPEDFATYYCQHHYESPLTFGGGTKVEIK

SEQ ID NO: 150 - protein, 107 aa, synthetic construct
  REGION 1..107: note = An artificially synthesized peptide sequence
DIQMTQSPSSLSASVGDRVTITCRTSENIYSFLAWYQQKPGKAPKLLIYNAKTLADGVPSRFSGSGSGTDFTLTISSLQPEDFATYYCQHHYESPLTFGGGTKVEIK

SEQ ID NO: 151 - protein, 447 aa, synthetic construct
  REGION 1..447: note = An artificially synthesized peptide sequence
QVQLVQSGAEVKKPGASVKVSCKASGYTFTGYIMNWVRQAPGQGLEWMGLINPYNGGTSYNQQFQDRVTITADESTSTAYMELSSLRSEDTAVYYCARDGYDDGPYTMDYWGQGTLVTVSSASTKGPSVFPLAPSSKSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 152 - protein, 214 aa, synthetic construct
  REGION 1..214: note = An artificially synthesized peptide sequence
DIQMTQSPSSLSASVGDRVTITCQTSEDIYSFLAWYQQKPGKAPKLLIYNAQTEAQGVPSRFSGSGSGTDFTLTISSLQPEDFATYYCQHHYESPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 326 aa, synthetic construct
  REGION 1..326: note = An artificially synthesized peptide sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 154 - protein, 326 aa, synthetic construct
  REGION 1..326: note = An artificially synthesized peptide sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 155 - protein, 119 aa, synthetic construct [sequence omitted]

SEQ ID NO: 156 - protein, 107 aa, synthetic construct [sequence omitted]

SEQ ID NO: 157 - protein, 445 aa, synthetic construct
  REGION 1..445: note = An artificially synthesized peptide sequence
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 158 - protein, 445 aa, synthetic construct
  REGION 1..445: note = An artificially synthesized peptide sequence
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 159 - protein, 449 aa, synthetic construct
  REGION 1..449: note = An artificially synthesized peptide sequence
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 445 aa, synthetic construct
  REGION 1..445: note = An artificially synthesized peptide sequence
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 161 - protein, 445 aa, synthetic construct
  REGION 1..445: note = An artificially synthesized peptide sequence
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 162 - protein, 214 aa, synthetic construct [sequence omitted]

SEQ ID NO: 163 - protein, 445 aa, synthetic construct
  REGION 1..445: note = An artificially synthesized peptide sequence
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 443 aa, synthetic construct
  REGION 1..443: note = An artificially synthesized peptide sequence
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 165 - protein, 267 aa, synthetic construct
  REGION 1..267: note = An artificially synthesized peptide sequence
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVEL

SEQ ID NO: 166 - protein, 99 aa, synthetic construct [sequence omitted]

SEQ ID NO: 167 - protein, 121 aa, synthetic construct
  REGION 1..121: note = An artificially synthesized peptide sequence
QVQLVQSGAEVKKPGASVKVSCKASGYTFTGYIMNWVRQAPGQGLEWMGLINPYNGGTSYNQQFQDRVTITADESTSTAYMELSSLRSEDTAVYYCARDGYDDGPYTMDYWGQGTLVTVSS

SEQ ID NO: 168 - protein, 107 aa, synthetic construct
  REGION 1..107: note = An artificially synthesized peptide sequence
DIQMTQSPSSLSASVGDRVTITCQTSEDIYSFLAWYQQKPGKAPKLLIYNAQTEAQGVPSRFSGSGSGTDFTLTISSLQPEDFATYYCQHHYESPLTFGGGTKVEIK

SEQ ID NO: 169 - protein, 11 aa, synthetic construct
  REGION 1..11: note = An artificially synthesized peptide sequence
QTSEDIYSFLA

SEQ ID NO: 170 - protein, 7 aa, synthetic construct
  REGION 1..7: note = An artificially synthesized peptide sequence
NAQTEAQ

SEQ ID NO: 171 - protein, 17 aa, synthetic construct
  REGION 1..17: note = An artificially synthesized peptide sequence
LINPYNGGTSYNQQFQD

SEQ ID NO: 172 - protein, 17 aa, synthetic construct
  REGION 1..17: note = An artificially synthesized peptide sequence
LINPYNGGTSYNDQFQD

SEQ ID NO: 173 - protein, 5 aa, synthetic construct
  REGION 1..5: note = An artificially synthesized peptide sequence
GYVMN

SEQ ID NO: 174 - protein, 5 aa, synthetic construct
  REGION 1..5: note = An artificially synthesized peptide sequence
GYIIN

SEQ ID NO: 175 - protein, 5 aa, synthetic construct
  REGION 1..5: note = An artificially synthesized peptide sequence
GYILN

SEQ ID NO: 176 - protein, 5 aa, synthetic construct
  REGION 1..5: note = An artificially synthesized peptide sequence
GYAMN

SEQ ID NO: 177 - protein, 17 aa, synthetic construct
  REGION 1..17: note = An artificially synthesized peptide sequence
LINPYNGGTDYNQKFKG

SEQ ID NO: 178 - protein, 17 aa, synthetic construct
  REGION 1..17: note = An artificially synthesized peptide sequence
LINPYNGGTSYNPKFKG

SEQ ID NO: 179 - protein, 12 aa, synthetic construct
  REGION 1..12: note = An artificially synthesized peptide sequence
DGLDDGPYTMDY

SEQ ID NO: 180 - protein, 12 aa, synthetic construct
  REGION 1..12: note = An artificially synthesized peptide sequence
DGYDDGPYTLDY

SEQ ID NO: 181 - protein, 12 aa, synthetic construct
  REGION 1..12: note = An artificially synthesized peptide sequence
DGYDDGPYTMEY

SEQ ID NO: 182 - protein, 12 aa, synthetic construct
  REGION 1..12: note = An artificially synthesized peptide sequence
DGYDDGPYTMDT

SEQ ID NO: 183 - protein, 12 aa, synthetic construct
  REGION 1..12: note = An artificially synthesized peptide sequence
DGYDDGPYTMDS

SEQ ID NO: 184 - protein, 12 aa, synthetic construct
  REGION 1..12: note = An artificially synthesized peptide sequence
DGYDDGPYTLET

SEQ ID NO: 185 - protein, 12 aa, synthetic construct
  REGION 1..12: note = An artificially synthesized peptide sequence
DGYDDGPYTMET

SEQ ID NO: 186 - protein, 12 aa, synthetic construct
  REGION 1..12: note = An artificially synthesized peptide sequence
DGLDDGPYTMET

SEQ ID NO: 187 - protein, 12 aa, synthetic construct
  REGION 1..12: note = An artificially synthesized peptide sequence
DGLDDGPYTMES

SEQ ID NO: 188 - protein, 11 aa, synthetic construct
  REGION 1..11: note = An artificially synthesized peptide sequence
RTSENIYRFLA

SEQ ID NO: 189 - protein, 11 aa, synthetic construct
  REGION 1..11: note = An artificially synthesized peptide sequence
RTSENIYSFVA

SEQ ID NO: 190 - protein, 11 aa, synthetic construct
  REGION 1..11: note = An artificially synthesized peptide sequence
RTSENIYRFVA

SEQ ID NO: 191 - protein, 11 aa, synthetic construct
  REGION 1..11: note = An artificially synthesized peptide sequence
RASENIYSFLA

SEQ ID NO: 192 - protein, 11 aa, synthetic construct
  REGION 1..11: note = An artificially synthesized peptide sequence
RSSENIYSFLA

SEQ ID NO: 193 - protein, 9 aa, synthetic construct
  REGION 1..9: note = An artificially synthesized peptide sequence
QHHYDSPLT

SEQ ID NO: 194 - protein, 9 aa, synthetic construct
  REGION 1..9: note = An artificially synthesized peptide sequence
QHHYEDPLT

SEQ ID NO: 195 - protein, 9 aa, synthetic construct
  REGION 1..9: note = An artificially synthesized peptide sequence
QHHYESPLF

SEQ ID NO: 196 - protein, 5 aa, synthetic construct
  REGION 1..5: note = An artificially synthesized peptide sequence
GYVLN

SEQ ID NO: 197 - protein, 17 aa, synthetic construct
  REGION 1..17: note = An artificially synthesized peptide sequence
LINPYNGGTDYNPQFQD

SEQ ID NO: 198 - protein, 17 aa, synthetic construct
  REGION 1..17: note = An artificially synthesized peptide sequence
LINPYNGGTDYNPQFQG

SEQ ID NO: 199 - protein, 17 aa, synthetic construct
  REGION 1..17: note = An artificially synthesized peptide sequence
LINPYNGGTSYNQQFQD

SEQ ID NO: 200 - protein, 11 aa, synthetic construct
  REGION 1..11: note = An artificially synthesized peptide sequence
QTSEDIYRFVA

SEQ ID NO: 201 - protein, 11 aa, synthetic construct
  REGION 1..11: note = An artificially synthesized peptide sequence
QTSEDIYSFVA

SEQ ID NO: 202 - protein, 11 aa, synthetic construct
  REGION 1..11: note = An artificially synthesized peptide sequence
QASEDIYSFVA

SEQ ID NO: 203 - protein, 11 aa, synthetic construct
  REGION 1..11: note = An artificially synthesized peptide sequence
QASEDIYSFLA

SEQ ID NO: 204 - protein, 121 aa, synthetic construct
  REGION 1..121: note = An artificially synthesized peptide sequence
QVQLVQSGAEVKKPGASVKVSCKASGYTFTGYVLNWVRQAPGQGLEWMGLINPYNGGTDYNPQFQDRVTITADESTSTAYMELSSLRSEDTAVYYCARDGYDDGPYTMDYWGQGTLVTVSS

SEQ ID NO: 205 - protein, 121 aa, synthetic construct
  REGION 1..121: note = An artificially synthesized peptide sequence
QVQLVQSGAEVKKPGASVKVSCKASGYTFTGYAMNWVRQAPGQGLEWMGLINPYNGGTDYNPQFQDRVTITADESTSTAYMELSSLRSEDTAVYYCARDGYDDGPYTMDYWGQGTLVTVSS

SEQ ID NO: 206 - protein, 121 aa, synthetic construct
  REGION 1..121: note = An artificially synthesized peptide sequence
QVQLVQSGAEVKKPGASVKVSCKASGYTFTGYVLNWVRQAPGQGLEWMGLINPYNGGTDYNPQFQDRVTITADESTSTAYMELSSLRSEDTAVYYCARDGYDDGPYTLETWGQGTLVTVSS

SEQ ID NO: 207 - protein, 121 aa, synthetic construct
  REGION 1..121: note = An artificially synthesized peptide sequence
QVQLVQSGAEVKKPGASVKVSCKASGYTFTGYIMNWVRQAPGQGLEWMGLINPYNGGTDYNPQFQDRVTITADESTSTAYMELSSLRSEDTAVYYCARDGYDDGPYTLETWGQGTLVTVSS

SEQ ID NO: 208 - protein, 121 aa, synthetic construct
  REGION 1..121: note = An artificially synthesized peptide sequence
QVQLVQSGAEVKKPGASVKVSCKASGYTFTGYAMNWVRQAPGQGLEWMGLINPYNGGTDYNPQFQDRVTITADESTSTAYMELSSLRSEDTAVYYCARDGYDDGPYTLETWGQGTLVTVSS

SEQ ID NO: 209 - protein, 121 aa, synthetic construct
  REGION 1..121: note = An artificially synthesized peptide sequence
QVQLVQSGAEVKKPGASVKVSCKASGYTFTGYVLNWVRQAPGQGLEWMGLINPYNGGTDYNPQFQDRVTITADKSTSTAYMELSSLRSEDTAVYYCARDGYDDGPYTLETWGQGTLVTVSS

SEQ ID NO: 210 - protein, 121 aa, synthetic construct
  REGION 1..121: note = An artificially synthesized peptide sequence
QVQLVQSGAEVKKPGASVKVSCKASGYTFTGYIMNWVRQAPGQGLEWMGLINPYNGGTDYNPQFQDRVTITADKSTSTAYMELSSLRSEDTAVYYCARDGYDDGPYTLETWGQGTLVTVSS

SEQ ID NO: 211 - protein, 121 aa, synthetic construct
  REGION 1..121: note = An artificially synthesized peptide sequence
QVQLVQSGAEVKKPGASVKVSCKASGYTFTGYAMNWVRQAPGQGLEWMGLINPYNGGTDYNPQFQDRVTITADKSTSTAYMELSSLRSEDTAVYYCARDGYDDGPYTLETWGQGTLVTVSS

SEQ ID NO: 212 - protein, 121 aa, synthetic construct
  REGION 1..121: note = An artificially synthesized peptide sequence
QVQLVQSGAEVKKPGASVKVSCKASGYTFTGYIMNWVRQAPGQGLEWMGLINPYNGGTDYNPQFQGRVTITADESTSTAYMELSSLRSEDTAVYYCARDGYDDGPYTLETWGQGTLVTVSS

SEQ ID NO: 213 - protein, 121 aa, synthetic construct
  REGION 1..121: note = An artificially synthesized peptide sequence
QVQLVQSGAEVKKPGASVKVSCKASGYTFTGYAMNWVRQAPGQGLEWMGLINPYNGGTDYNPQFQGRVTITADESTSTAYMELSSLRSEDTAVYYCARDGYDDGPYTLETWGQGTLVTVSS

SEQ ID NO: 214 - protein, 121 aa, synthetic construct
  REGION 1..121: note = An artificially synthesized peptide sequence
QVQLVQSGAEVKKPGASVKVSCKASGYTFTGYIMNWVRQAPGQGLEWMGLINPYNGGTDYNPQFQGRVTITADKSTSTAYMELSSLRSEDTAVYYCARDGYDDGPYTLETWGQGTLVTVSS

SEQ ID NO: 215 - protein, 121 aa, synthetic construct
  REGION 1..121: note = An artificially synthesized peptide sequence
QVQLVQSGAEVKKPGASVKVSCKASGYTFTGYAMNWVRQAPGQGLEWMGLINPYNGGTDYNPQFQGRVTITADKSTSTAYMELSSLRSEDTAVYYCARDGYDDGPYTLETWGQGTLVTVSS

SEQ ID NO: 216 - protein, 121 aa, synthetic construct
  REGION 1..121: note = An artificially synthesized peptide sequence
QVQLVQSGAEVKKPGASVKVSCKASGYTFTGYIMNWVRQAPGQGLEWMGLINPYNGGTSYNQQFQDRVTITADESTSTAYMELSSLRSEDTAVYYCARDGYDDGPYTLETWGQGTLVTVSS

SEQ ID NO: 217 - protein, 121 aa, synthetic construct
  REGION 1..121: note = An artificially synthesized peptide sequence
QVQLVQSGAEVKKPGASVKVSCKASGYTFTGYIMNWVRQAPGQGLEWMGLINPYNGGTSYNQQFQDRVTITADKSTSTAYMELSSLRSEDTAVYYCARDGYDDGPYTLETWGQGTLVTVSS

SEQ ID NO: 218 - protein, 107 aa, synthetic construct
  REGION 1..107: note = An artificially synthesized peptide sequence
DIQMTQSPSSLSASVGDRVTITCQTSEDIYRFVAWYQQKPGKAPKLLIYNAQTEAQGVPSRFSGSGSGTDFTLTISSLQPEDFATYYCQHHYDSPLTFGGGTKVEIK

SEQ ID NO: 219 - protein, 107 aa, synthetic construct
  REGION 1..107: note = An artificially synthesized peptide sequence
DIQMTQSPSSLSASVGDRVTITCQTSEDIYSFVAWYQQKPGKAPKLLIYNAQTEAQGVPSRFSGSGSGTDFTLTISSLQPEDFATYYCQHHYDSPLTFGGGTKVEIK

SEQ ID NO: 220 - protein, 107 aa, synthetic construct
  REGION 1..107: note = An artificially synthesized peptide sequence
DIQMTQSPSSLSASVGDRVTITCQASEDIYSFVAWYQQKPGKAPKLLIYNAQTEAQGVPSRFSGSGSGTDFTLTISSLQPEDFATYYCQHHYDSPLTFGGGTKVEIK

SEQ ID NO: 221 - protein, 107 aa, synthetic construct
  REGION 1..107: note = An artificially synthesized peptide sequence
DIQMTQSPSSLSASVGDRVTITCQASEDIYSFLAWYQQKPGKAPKLLIYNAQTEAQGVPSRFSGSGSGTDFTLTISSLQPEDFATYYCQHHYDSPLTFGGGTKVEIK

SEQ ID NO: 222 - protein, 445 aa, synthetic construct
  REGION 1..445: note = An artificially synthesized peptide sequence
QVQLVQSGAEVKKPGASVKVSCKASGYTFTGYVLNWVRQAPGQGLEWMGLINPYNGGTDYNPQFQDRVTITADESTSTAYMELSSLRSEDTAVYYCARDGYDDGPYTMDYWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 223 - protein, 445 aa, synthetic construct
  REGION 1..445: note = An artificially synthesized peptide sequence
QVQLVQSGAEVKKPGASVKVSCKASGYTFTGYAMNWVRQAPGQGLEWMGLINPYNGGTDYNPQFQDRVTITADESTSTAYMELSSLRSEDTAVYYCARDGYDDGPYTMDYWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 224 - protein, 445 aa, synthetic construct
  REGION 1..445: note = An artificially synthesized peptide sequence
QVQLVQSGAEVKKPGASVKVSCKASGYTFTGYVLNWVRQAPGQGLEWMGLINPYNGGTDYNPQFQDRVTITADESTSTAYMELSSLRSEDTAVYYCARDGYDDGPYTLETWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 225 - protein, 445 aa, synthetic construct
  REGION 1..445: note = An artificially synthesized peptide sequence
QVQLVQSGAEVKKPGASVKVSCKASGYTFTGYIMNWVRQAPGQGLEWMGLINPYNGGTDYNPQFQDRVTITADESTSTAYMELSSLRSEDTAVYYCARDGYDDGPYTLETWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 226 - protein, 445 aa, synthetic construct
  REGION 1..445: note = An artificially synthesized peptide sequence
QVQLVQSGAEVKKPGASVKVSCKASGYTFTGYAMNWVRQAPGQGLEWMGLINPYNGGTDYNPQFQDRVTITADESTSTAYMELSSLRSEDTAVYYCARDGYDDGPYTLETWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 227 - protein, 445 aa, synthetic construct
  REGION 1..445: note = An artificially synthesized peptide sequence
QVQLVQSGAEVKKPGASVKVSCKASGYTFTGYVLNWVRQAPGQGLEWMGLINPYNGGTDYNPQFQDRVTITADKSTSTAYMELSSLRSEDTAVYYCARDGYDDGPYTLETWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 228 - protein, 445 aa, synthetic construct
  REGION 1..445: note = An artificially synthesized peptide sequence
QVQLVQSGAEVKKPGASVKVSCKASGYTFTGYIMNWVRQAPGQGLEWMGLINPYNGGTDYNPQFQDRVTITADKSTSTAYMELSSLRSEDTAVYYCARDGYDDGPYTLETWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 229 - protein, 445 aa, synthetic construct
  REGION 1..445: note = An artificially synthesized peptide sequence
QVQLVQSGAEVKKPGASVKVSCKASGYTFTGYAMNWVRQAPGQGLEWMGLINPYNGGTDYNPQFQDRVTITADKSTSTAYMELSSLRSEDTAVYYCARDGYDDGPYTLETWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 230 - protein, 445 aa, synthetic construct
  REGION 1..445: note = An artificially synthesized peptide sequence
QVQLVQSGAEVKKPGASVKVSCKASGYTFTGYIMNWVRQAPGQGLEWMGLINPYNGGTDYNPQFQGRVTITADESTSTAYMELSSLRSEDTAVYYCARDGYDDGPYTLETWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 231 - protein, 445 aa, synthetic construct
  REGION 1..445: note = An artificially synthesized peptide sequence
QVQLVQSGAEVKKPGASVKVSCKASGYTFTGYAMNWVRQAPGQGLEWMGLINPYNGGTDYNPQFQGRVTITADESTSTAYMELSSLRSEDTAVYYCARDGYDDGPYTLETWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 232 - protein, 445 aa, synthetic construct
  REGION 1..445: note = An artificially synthesized peptide sequence
QVQLVQSGAEVKKPGASVKVSCKASGYTFTGYIMNWVRQAPGQGLEWMGLINPYNGGTDYNPQFQGRVTITADKSTSTAYMELSSLRSEDTAVYYCARDGYDDGPYTLETWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 233 - protein, 445 aa, synthetic construct
  REGION 1..445: note = An artificially synthesized peptide sequence
QVQLVQSGAEVKKPGASVKVSCKASGYTFTGYAMNWVRQAPGQGLEWMGLINPYNGGTDYNPQFQGRVTITADKSTSTAYMELSSLRSEDTAVYYCARDGYDDGPYTLETWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 234 - protein, 445 aa, synthetic construct
  REGION 1..445: note = An artificially synthesized peptide sequence
QVQLVQSGAEVKKPGASVKVSCKASGYTFTGYIMNWVRQAPGQGLEWMGLINPYNGGTSYNQQFQDRVTITADESTSTAYMELSSLRSEDTAVYYCARDGYDDGPYTLETWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 235 - protein, 445 aa, synthetic construct
  REGION 1..445: note = An artificially synthesized peptide sequence
QVQLVQSGAEVKKPGASVKVSCKASGYTFTGYIMNWVRQAPGQGLEWMGLINPYNGGTSYNQQFQDRVTITADKSTSTAYMELSSLRSEDTAVYYCARDGYDDGPYTLETWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 236 - protein, 214 aa, synthetic construct
  REGION 1..214: note = An artificially synthesized peptide sequence
DIQMTQSPSSLSASVGDRVTITCQTSEDIYRFVAWYQQKPGKAPKLLIYNAQTEAQGVPSRFSGSGSGTDFTLTISSLQPEDFATYYCQHHYDSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - protein, 214 aa, synthetic construct
  REGION 1..214: note = An artificially synthesized peptide sequence
DIQMTQSPSSLSASVGDRVTITCQTSEDIYSFVAWYQQKPGKAPKLLIYNAQTEAQGVPSRFSGSGSGTDFTLTISSLQPEDFATYYCQHHYDSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 214 aa, synthetic construct
  REGION 1..214: note = An artificially synthesized peptide sequence
DIQMTQSPSSLSASVGDRVTITCQASEDIYSFVAWYQQKPGKAPKLLIYNAQTEAQGVPSRFSGSGSGTDFTLTISSLQPEDFATYYCQHHYDSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 239 - protein, 214 aa, synthetic construct
  REGION 1..214: note = An artificially synthesized peptide sequence
DIQMTQSPSSLSASVGDRVTITCQASEDIYSFLAWYQQKPGKAPKLLIYNAQTEAQGVPSRFSGSGSGTDFTLTISSLQPEDFATYYCQHHYDSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 240 - protein, 445 aa, synthetic construct
  REGION 1..445: note = An artificially synthesized peptide sequence
QVQLVQSGAEVKKPGASVKVSCKASGYTFTGYIMNWVRQAPGQGLEWMGLINPYNGGTSYNQQFQDRVTITADESTSTAYMELSSLRSEDTAVYYCARDGYDDGPYTMDYWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 241 - protein, 447 aa, synthetic construct
  REGION 1..447: note = An artificially synthesized peptide sequence
QVQLVQSGAEVKKPGASVKVSCKASGYTFTGYIMNWVRQAPGQGLEWMGLINPYNGGTSYNQQFQDRVTITADESTSTAYMELSSLRSEDTAVYYCARDGYDDGPYTMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 242 - protein, 447 aa, synthetic construct
  REGION 1..447: note = An artificially synthesized peptide sequence
QVQLVQSGAEVKKPGASVKVSCKASGYTFTGYIMNWVRQAPGQGLEWMGLINPYNGGTSYNQKFKGRVTITADESTSTAYMELSSLRSEDTAVYYCARDGYDDGPYTLETWGQGTLVTVSSASTKGPSVFPLAPSSKSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 243 - protein, 447 aa, synthetic construct
  REGION 1..447: note = An artificially synthesized peptide sequence
QVQLVQSGAEVKKPGASVKVSCKASGYTFTGYIMNWVRQAPGQGLEWMGLINPYNGGTSYNQKFKGRVTITADESTSTAYMELSSLRSEDTAVYYCARDGYDDGPYTMETWGQGTLVTVSSASTKGPSVFPLAPSSKSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 244 - protein, 447 aa, synthetic construct
  REGION 1..447: note = An artificially synthesized peptide sequence
QVQLVQSGAEVKKPGASVKVSCKASGYTFTGYIMNWVRQAPGQGLEWMGLINPYNGGTSYNQKFKGRVTITADESTSTAYMELSSLRSEDTAVYYCARDGLDDGPYTMETWGQGTLVTVSSASTKGPSVFPLAPSSKSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 245 - protein, 447 aa, synthetic construct
  REGION 1..447: note = An artificially synthesized peptide sequence
QVQLVQSGAEVKKPGASVKVSCKASGYTFTGYIMNWVRQAPGQGLEWMGLINPYNGGTSYNQKFKGRVTITADESTSTAYMELSSLRSEDTAVYYCARDGLDDGPYTMESWGQGTLVTVSSASTKGPSVFPLAPSSKSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 246 - protein, 447 aa, synthetic construct
  REGION 1..447: note = An artificially synthesized peptide sequence
QVQLVQSGAEVKKPGASVKVSCKASGYTFTGYVMNWVRQAPGQGLEWMGLINPYNGGTSYNQKFKGRVTITADESTSTAYMELSSLRSEDTAVYYCARDGYDDGPYTMDYWGQGTLVTVSSASTKGPSVFPLAPSSKSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 247 - protein, 447 aa, synthetic construct
  REGION 1..447: note = An artificially synthesized peptide sequence
QVQLVQSGAEVKKPGASVKVSCKASGYTFTGYIINWVRQAPGQGLEWMGLINPYNGGTSYNQKFKGRVTITADESTSTAYMELSSLRSEDTAVYYCARDGYDDGPYTMDYWGQGTLVTVSSASTKGPSVFPLAPSSKSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 248 - protein, 447 aa, synthetic construct
  REGION 1..447: note = An artificially synthesized peptide sequence
QVQLVQSGAEVKKPGASVKVSCKASGYTFTGYILNWVRQAPGQGLEWMGLINPYNGGTSYNQKFKGRVTITADESTSTAYMELSSLRSEDTAVYYCARDGYDDGPYTMDYWGQGTLVTVSSASTKGPSVFPLAPSSKSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 249 - protein, 447 aa, synthetic construct
  REGION 1..447: note = An artificially synthesized peptide sequence
QVQLVQSGAEVKKPGASVKVSCKASGYTFTGYIMNWVRQAPGQGLEWMGLINPYNGGTDYNQKFKGRVTITADESTSTAYMELSSLRSEDTAVYYCARDGYDDGPYTMDYWGQGTLVTVSSASTKGPSVFPLAPSSKSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 250 - protein, 447 aa, synthetic construct
  REGION 1..447: note = An artificially synthesized peptide sequence
QVQLVQSGAEVKKPGASVKVSCKASGYTFTGYIMNWVRQAPGQGLEWMGLINPYNGGTSYNPKFKGRVTITADESTSTAYMELSSLRSEDTAVYYCARDGYDDGPYTMDYWGQGTLVTVSSASTKGPSVFPLAPSSKSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 251 - protein, 214 aa, synthetic construct
  REGION 1..214: note = An artificially synthesized peptide sequence
DIQMTQSPSSLSASVGDRVTITCRTSENIYRFLAWYQQKPGKAPKLLIYNAKTLAKGVPSRFSGSGSGTDFTLTISSLQPEDFATYYCQHHYESPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 252 - protein, 214 aa, synthetic construct
  REGION 1..214: note = An artificially synthesized peptide sequence
DIQMTQSPSSLSASVGDRVTITCRTSENIYRFVAWYQQKPGKAPKLLIYNAKTLAKGVPSRFSGSGSGTDFTLTISSLQPEDFATYYCQHHYESPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 253 - protein, 214 aa, synthetic construct
  REGION 1..214: note = An artificially synthesized peptide sequence
DIQMTQSPSSLSASVGDRVTITCRTSENIYSFLAWYQQKPGKAPKLLIYNAKTLAKGVPSRFSGSGSGTDFTLTISSLQPEDFATYYCQHHYDSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 254 - protein, 214 aa, synthetic construct
  REGION 1..214: note = An artificially synthesized peptide sequence
DIQMTQSPSSLSASVGDRVTITCRTSENIYSFLAWYQQKPGKAPKLLIYNAKTLAKGVPSRFSGSGSGTDFTLTISSLQPEDFATYYCQHHYEDPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 255 - protein, 447 aa, synthetic construct
  REGION 1..447: note = An artificially synthesized peptide sequence
QVQLVQSGAEVKKPGASVKVSCKASGYTFTGYVINWVRQAPGQGLEWMGLINPYNGGTDYNQKFKGRVTITADESTSTAYMELSSLRSEDTAVYYCARDGYDDGPYTMDYWGQGTLVTVSSASTKGPSVFPLAPSSKSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 256 - protein, 214 aa, synthetic construct
  REGION 1..214: note = An artificially synthesized peptide sequence
DIQMTQSPSSLSASVGDRVTITCQTSENIYRFVAWYQQKPGKAPKLLIYNAKTLAKGVPSRFSGSGSGTDFTLTISSLQPEDFATYYCQHHYDSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 257 - protein, 445 aa, synthetic construct
  REGION 1..445: note = An artificially synthesized peptide sequence
QVQLVQSGAEVKKPGASVKVSCKASGYTFTGYIMNWVRQAPGQGLEWMGLINPYNGGTSYNQKFKGRVTITADKSTSTAYMELSSLRSEDTAVYYCARDGYDDGPYTMDYWGQGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHNHYTQKSLSLSP

SEQ ID NO: 258 - protein, 530 aa, Homo sapiens
MMWTWALWMLPSLCKFSLAALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSSALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMASTSAGGTNGTSINFKTLSHHHHHHEQKLISEEDL

SEQ ID NO: 259 - protein, 517 aa, Mus musculus
MWTLALWAFSFLCKFSLAVLPTKPENISCVFYFDRNLTCTWRPEKETNDTSYIVTLTYSYGKSNYSDNATEASYSFPRSCAMPPDICSVEVQAQNGDGKVKSDITYWHLISIAKTEPPIILSVNPICNRMFQIQWKPREKTRGFPLVCMLRFRTVNSSRWTEVNFENCKQVCNLTGLQAFTEYVLALRFRFNDSRYWSKWSKEETRVTMEEVPHVLDLWRILEPADMNGDRKVRLLWKKARGAPVLEKTFGYHIQYFAENSTNLTEINNITTQQYELLLMSQAHSVSVTSFNSLGKSQETILRIPDVHEKTFQYIKSMQAYIAEPLLVVNWQSSIPAVDTWIVEWLPEAAMSKFPALSWESVSQVTNWTIEQDKLKPFTCYNISVYPVLGHRVGEPYSIQAYAKEGTPLKGPETRVENIGLRTATITWKEIPKSARNGFINNYTVFYQAEGGKELSKTVNSHALQCDLESLTRRTSYTVWVMASTRAGGTNGVRINFKTLSHHHHHHEQKLISEEDL

SEQ ID NO: 260 - protein, 531 aa, synthetic construct
  REGION 1..531: note = An artificially synthesized peptide sequence
MMWTWALWMLPSLCKFSLAALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPHVLDLWRILEPADMNGDRKVRLLWKKARGAPVLEKTFGYHIQYFAENSTNLTEINNITTQQYELLLMSQAHSVSVTSFNSLGKSQETILRIPDVHEKTFQYIKSMQAYIAEPLLVVNWQSSIPAVDTWIVEWLPEAAMSKFPALSWESVSQVTNWTIEQDKLKPFTCYNISVYPVLGHRVGEPYSIQAYAKEGTPLKGPETRVENIGLRTATITWKEIPKSARNGFINNYTVFYQAEGGKELSKTVNSHALQCDLESLTRRTSYTVWVMASTRAGGTNGVRINFKTLSHHHHHHEQKLISEEDL

SEQ ID NO: 261 - protein, 516 aa, synthetic construct
  REGION 1..516: note = An artificially synthesized peptide sequence
MWTLALWAFSFLCKFSLAVLPTKPENISCVFYFDRNLTCTWRPEKETNDTSYIVTLTYSYGKSNYSDNATEASYSFPRSCAMPPDICSVEVQAQNGDGKVKSDITYWHLISIAKTEPPIILSVNPICNRMFQIQWKPREKTRGFPLVCMLRFRTVNSSRWTEVNFENCKQVCNLTGLQAFTEYVLALRFRFNDSRYWSKWSKEETRVTMEEVPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSSALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMASTSAGGTNGTSINFKTLSHHHHHHEQKLISEEDL

SEQ ID NO: 262 - protein, 524 aa, synthetic construct
  REGION 1..524: note = An artificially synthesized peptide sequence
MMWTWALWMLPSLCKFSLAALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPIILSVNPICNRMFQIQWKPREKTRGFPLVCMLRFRTVNSSRWTEVNFENCKQVCNLTGLQAFTEYVLALRFRFNDSRYWSKWSKEETRVTMEEVPHVLDLWRILEPADMNGDRKVRLLWKKARGAPVLEKTFGYHIQYFAENSTNLTEINNITTQQYELLLMSQAHSVSVTSFNSLGKSQETILRIPDVHEKTFQYIKSMQAYIAEPLLVVNWQSSIPAVDTWIVEWLPEAAMSKFPALSWESVSQVTNWTIEQDKLKPFTCYNISVYPVLGHRVGEPYSIQAYAKEGTPLKGPETRVENIGLRTATITWKEIPKSARNGFINNYTVFYQAEGGKELSKTVNSHALQCDLESLTRRTSYTVWVMASTRAGGTNGVRINFKTLSHHHHHHEQKLISEEDL

SEQ ID NO: 263 - protein, 524 aa, synthetic construct
  REGION 1..524: note = An artificially synthesized peptide sequence
MWTLALWAFSFLCKFSLAVLPTKPENISCVFYFDRNLTCTWRPEKETNDTSYIVTLTYSYGKSNYSDNATEASYSFPRSCAMPPDICSVEVQAQNGDGKVKSDITYWHLISIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPHVLDLWRILEPADMNGDRKVRLLWKKARGAPVLEKTFGYHIQYFAENSTNLTEINNITTQQYELLLMSQAHSVSVTSFNSLGKSQETILRIPDVHEKTFQYIKSMQAYIAEPLLVVNWQSSIPAVDTWIVEWLPEAAMSKFPALSWESVSQVTNWTIEQDKLKPFTCYNISVYPVLGHRVGEPYSIQAYAKEGTPLKGPETRVENIGLRTATITWKEIPKSARNGFINNYTVFYQAEGGKELSKTVNSHALQCDLESLTRRTSYTVWVMASTRAGGTNGVRINFKTLSHHHHHHEQKLISEEDL

SEQ ID NO: 264 - protein, 523 aa, synthetic construct
  REGION 1..523: note = An artificially synthesized peptide sequence
MWTLALWAFSFLCKFSLAVLPTKPENISCVFYFDRNLTCTWRPEKETNDTSYIVTLTYSYGKSNYSDNATEASYSFPRSCAMPPDICSVEVQAQNGDGKVKSDITYWHLISIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSSALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMASTSAGGTNGTSINFKTLSHHHHHHEQKLISEEDL

SEQ ID NO: 265 - protein, 12 aa, synthetic construct
  REGION 1..12: note = An artificially synthesized peptide sequence
DGYDDGPYTMDY

SEQ ID NO: 266 - protein, 12 aa, synthetic construct
  REGION 1..12: note = An artificially synthesized peptide sequence
DGYDDGPYTLET

SEQ ID NO: 267 - protein, 12 aa, synthetic construct
  REGION 1..12: note = An artificially synthesized peptide sequence
DGYDDGPYTMET

SEQ ID NO: 268 - protein, 12 aa, synthetic construct
  REGION 1..12: note = An artificially synthesized peptide sequence
DGLDDGPYTMET

SEQ ID NO: 269 - protein, 12 aa, synthetic construct
  REGION 1..12: note = An artificially synthesized peptide sequence
DGLDDGPYTMES

SEQ ID NO: 270 - protein, 5 aa, synthetic construct
  REGION 1..5: note = An artificially synthesized peptide sequence
GYIMN

SEQ ID NO: 271 - protein, 17 aa, synthetic construct
  REGION 1..17: note = An artificially synthesized peptide sequence
LINPYNGGTSYNQKFKG

SEQ ID NO: 272 - protein, 5 aa, synthetic construct
  REGION 1..5: note = An artificially synthesized peptide sequence
GYVMN

SEQ ID NO: 273 - protein, 5 aa, synthetic construct
  REGION 1..5: note = An artificially synthesized peptide sequence
GYIIN

SEQ ID NO: 274 - protein, 5 aa, synthetic construct
  REGION 1..5: note = An artificially synthesized peptide sequence
GYILN

SEQ ID NO: 275 - protein, 17 aa, synthetic construct
  REGION 1..17: note = An artificially synthesized peptide sequence
LINPYNGGTDYNQKFKG

SEQ ID NO: 276 - protein, 17 aa, synthetic construct
  REGION 1..17: note = An artificially synthesized peptide sequence
LINPYNGGTSYNPKFKG

SEQ ID NO: 277 - protein, 11 aa, synthetic construct
  REGION 1..11: note = An artificially synthesized peptide sequence
RTSENIYSFLA

SEQ ID NO: 278 - protein, 9 aa, synthetic construct
  REGION 1..9: note = An artificially synthesized peptide sequence
QHHYESPLT

SEQ ID NO: 279 - protein, 11 aa, synthetic construct
  REGION 1..11: note = An artificially synthesized peptide sequence
RTSENIYRFLA

SEQ ID NO: 280 - protein, 11 aa, synthetic construct
  REGION 1..11: note = An artificially synthesized peptide sequence
RTSENIYRFVA

SEQ ID NO: 281 - protein, 9 aa, synthetic construct
  REGION 1..9: note = An artificially synthesized peptide sequence
QHHYDSPLT

SEQ ID NO: 282 - protein, 9 aa, synthetic construct
  REGION 1..9: note = An artificially synthesized peptide sequence
QHHYEDPLT

SEQ ID NO: 283 - protein, 9 aa, synthetic construct
  REGION 1..9: note = An artificially synthesized peptide sequence
QHHTESPLF

SEQ ID NO: 284 - protein, 5 aa, synthetic construct
  REGION 1..5: note = An artificially synthesized peptide sequence
GYAMN

SEQ ID NO: 285 - protein, 11 aa, synthetic construct
  REGION 1..11: note = An artificially synthesized peptide sequence
RASENIYSFLA

SEQ ID NO: 286 - protein, 11 aa, synthetic construct
  REGION 1..11: note = An artificially synthesized peptide sequence
RSSENIYSFLA

SEQ ID NO: 287 - protein, 16 aa, synthetic construct
  REGION 1..16: note = An artificially synthesized sequence
HHHHHHEQKLISEEDL